(19) United States
(12) Patent Application Publication
(10) Pub. No.: US 2026/0245653 A1
(43) Pub. Date: Aug. 20, 2026

(54) METHOD FOR DETERMINING SURFACE PROTRUDING AMINO ACIDS OF A HUMAN LEUKOCYTE ANTIGEN (HLA) PROTEIN

(71) Applicant: PIRCHE AG, Grünwald (DE)

(72) Inventor: Matthias Niemann, Berlin (DE)

(21) Appl. No.: 19/469,653

(22) PCT Filed: Mar. 27, 2024
(86) PCT No.: PCT/EP2024/058372

§ 371 Date: Sep. 26, 2025

(30) Foreign Application Priority Data
Mar. 27, 2023 (EP) 23164420.4
May 16, 2023 (EP) 23173788.3

(51) Int. Cl. G16B 15/20; G16B 40/00; G16H 50/70

(52) U.S. Cl. CPC G16B 15/20; G16B 40/00; G16H 50/70

(57) ABSTRACT

A method for determining an amino acid residue protruding from the surface of a human leukocyte antigen (HLA) protein structure, including: (i) employing an ellipsoid-fitting algorithm to a subset of atoms of an HLA protein structure that are in spatial proximity to each other, (ii) repeating step (i) for multiple subsets of atoms of the HLA protein structure, thereby creating multiple fitted ellipsoids, and (iii) determining whether an amino acid of an HLA protein structure is surface protruding by considering the spatial distribution of atoms of any given amino acid residue relative to the position of the multiple fitted ellipsoids.

The invention relates to the fields of biology, bioinformatics, immunology and computer implementation of biological methods.

The invention relates to a computer-implemented method for determining an amino acid residue protruding from the surface of a human leukocyte antigen (HLA) protein structure. In embodiments, the method comprises: (i) employing an ellipsoid-fitting algorithm to a subset of atoms of an HLA protein structure that are in spatial proximity to each other, (ii) repeating step (i) for multiple subsets of atoms of the HLA protein structure, thereby creating multiple fitted ellipsoids, and (iii) determining whether an amino acid of an HLA protein structure is surface protruding by considering the spatial distribution of atoms of any given amino acid residue relative to the position of the multiple fitted ellipsoids.

The invention further relates to a computer-implemented method for producing a database of predicted surface-protruding residues for multiple human leukocyte antigen (HLA) proteins. IN embodiment, the method comprises: a. Providing one or more HLA protein amino acid sequences, for which a protein structure has been experimentally determined, b. Predicting HLA protein structure of one or more HLA proteins, c. Supplementing the predicted HLA protein structures of b. with an HLA-bound peptide, if the structure of b. does not exhibit an HLA-bound peptide, d. Determining surface-protruding residues of each HLA protein structure of a. and c., and e. generating a database of surface protruding residues for the HLA proteins of a. and c. and HLA proteins additional to those of a. to c., comprising employing a neural network trained on amino acids sequences of the HLA proteins of a. and c. and the corresponding surface-protruding residues determined in d.

In another aspect the invention relates to a computer-implemented method for predicting a human leukocyte antigen (HLA) B-cell epitope that is mismatched between two or more subjects. IN embodiments, the method comprises: Determining one or more mismatched amino acids of an HLA protein between two or more subjects, and determining whether said mismatched amino acids are predicted to be protruding from a surface of an HLA structure, wherein a mismatched amino acid predicted to be surface-protruding indicates a human leukocyte antigen (HLA) B-cell epitope that is mismatched between two or more subjects.

In another aspect the invention relates to a computer-implemented method for selecting transplantation material for allogeneic transplantation between subjects with one or more human leukocyte antigen (HLA) mismatches. In embodiments, the method comprises assessing whether one or more human leukocyte antigen (HLA) B-cell epitopes are mismatched between said subjects according to the present method, wherein the number of determined mismatched HLA B-cell epitopes positively correlates with an antibody immune response against a corresponding mismatched HLA protein and/or a likelihood of an adverse immune reaction after transplantation.

### BACKGROUND OF THE INVENTION

Hematopoietic Stem Cell Transplantation (HSCT) is one example of a quickly growing therapeutic approach. The major limiting factor of HSCT remains the risk of graft-versus-host disease (GVHD), and since the number of patients receiving HSCT is expected to increase, the provision of novel approaches to prevent GVHD must be accelerated. To overcome the risk of GVHD, patients are preferably transplanted with a donor that is completely matched for all HLA alleles. However, due to heterogeneity of HLA molecules in the population, these completely matched donors are not available for approximately 40% of patients. With increasing diversity of populations this issue become more pronounced, and it is expected to find optimal donors for even fewer patients. When a completely matched donor is not available, a clinician often has to face the difficult decision to choose the least harmful donor out of the mismatched donors (e.g., the one that carries the lowest GVHD risk). Similar problems face clinicians before selecting transplantation material for blood products, such as platelets, or cord blood or cord blood cell transplantations, kidney transplantations, or other transplantations at risk of side effects caused by unwanted alloreactivity.

Allorecognition in transplantation has been shown to be a major challenge in long term kidney graft survival. [1,2] Several approaches have been developed in recent years to predict a recipient's likelihood of anti-donor human leukocyte antigen (HLA) antibody recognition in order to achieve histocompatibility. [3] Polymorphic amino acid positions located on the HLA protein's surface have been identified manually for a limited set of experimental crystal structures. These polymorphisms are defined as Eplets upon which the HLA Matchmaker algorithm is based. [4] Later concepts such as EMS3D or HLA-EMMA formalized HLA protein differences further by considering electrostatic dissimilarity or solvent accessibility.

The HLA-EMMA algorithm however appears to be disadvantageous by limitation to an analysis of experimentally determined HLA structures, as solvent accessible polymorphic amino acid positions were determined using only publicly available crystal structures and solvent accessibility prediction tools. However, no mention is made of additionally using predicted HLA structures to complement and expand the analysis. The HLA-EMMA algorithm also appears to not use structures supplemented with HLA-bound peptides, thus reducing the practical accuracy and applicability of the method. Furthermore, the HLA-EMMA method appears not to employ artificial intelligence or machine learning to predict solvent accessible surface residues based on HLA sequence alone, after being trained on HLA sequences, structures and predicted solvent accessible surface residues. HLA-EMMA appears to only use experimentally established structures to assess solvent accessible surface residues and is thus limited to those HLA proteins for which structures have been solved. [5-7]

The recently proposed Snowflake algorithm incorporates HLA protein-specific structure differences when analyzing amino acid mismatches' surface area. [8,9]

It has been suggested by Duquesnoy et al. that predicted protrusion of Eplets may add value to identifying their antibody accessibility. [10] Therefore, Duquesnoy et al applied the ElliPro software that has been developed by Ponomarenko et al. [11] ElliPro implements a mathematical approach of Thornton et al., that fits amino acids' atom positions in ellipsoids and ranks their distance from the ellipsoid's center considering the ellipsoid's axes proportionally. [12,13] However, the ElliPro approach aims to fit all atoms of the amino acids of a protein into one ellipsoid. This approach constitutes a disadvantage for the analysis of amino acids or proteins that do not naturally comprise an ellipsoid shape. In large non-elliptic structures, ElliPro allows identifying protruding regions of the molecule (macroscopic), but fails to identify locally protruding amino acids of the protein's substructures (microscopic). Considering the characteristic shape of HLA proteins, ElliPro is considered to be insensitive for complex folding patterns like the HLA's helix structures at the antigen binding domain, considering the helical structures being surface-protruding even though the folding pattern is known to alternate between inward and outward facing amino acids.

Despite currently available methods, there still exists a need for methods with high precision and reliability to predict increased risk of unwanted immune response or even failed transplantation when non-HLA-matched donor material is used for transplantation, such that the development of donor-specific antibodies, GVHD, antibody- or T-cell-mediated rejection (AMR/TCMR), susceptibility to infections, graft loss, and/or increased mortality after organ or tissue transplantation can be effectively prevented or the risk thereof reduced.

### SUMMARY OF THE INVENTION

In view of the prior art, the technical problem underlying the present invention is the provision of improved or alternative computer-implemented means for the assessment of histocompatibility between patients and organ donors. A further technical problem may be considered as the provision of improved or alternative computer-implemented means for assessing mismatched HLA B cell epitopes and their predicted role in immune responses in the context of transplantation medicine. A further technical problem may be considered as the provision of improved or alternative computer-implemented means for assessing whether any given amino acid or protein region of an HLA molecule is accessible as a B cell or antibody epitope. A further technical problem may be considered as the provision of improved or alternative computer-implemented means for assessing whether any given amino acid or protein region of an HLA molecule protrudes from the surface of its structure.

This problem is solved by the features of the independent claims. Preferred embodiments of the present invention are provided by the dependent claims.

The invention therefore relates to a computer-implemented method for determining an amino acid residue protruding from the surface of a human leukocyte antigen (HLA) protein structure.

The invention therefore relates to a computer-implemented method for determining an amino acid residue protruding from the surface of a human leukocyte antigen (HLA) protein structure, comprising:
(i) employing an ellipsoid-fitting algorithm to a subset of atoms of an HLA protein structure, that are in spatial proximity to each other,
(ii) repeating step (i) for multiple subsets of atoms of the HLA protein structure thereby creating multiple fitted ellipsoids, and
(iii) determining whether an amino acid of an HLA protein structure is surface protruding by considering the spatial distribution of atoms of any given amino acid residue relative to the position of the multiple fitted ellipsoids.

A surprising effect and significant advantage of the present method over prior art methods, such as the ElliPro approach, is that the ellipsoid fitting is independent of the spatial structure of an amino acid or of the entire protein that is analyzed. This surprising effect results from the features that the ellipsoid fitting of step (i) is performed on only a subset of amino acids or atoms which is then repeated (step (ii)) for multiple subsets of atoms of one or more amino acids of a protein structure, or of the entire structure of a protein, such as an HLA protein structure. In embodiments, the present approach facilitates the “scanning” of a protein structure through the repetitive fitting of numerous ellipsoids to a protein structure's atoms, or to subsets of its atoms, thereby determining numerous so called “protrusion ranks” for multiple atoms or for each atom of a protein structure. Thereby the protrusion of an amino acid of a protein or of a sub-structure of a protein (e.g., a part of a protein) can be determined by considering multiple protrusion ranks of numerous atoms or for each atom of a protein structure or sub-structure.

Therefore, a high protrusion rank of an amino acid residue indicates a profound protrusion of this residue from the protein's surface. Therefore, in embodiments, a high protrusion rank of an amino acid residue that is mismatched between a donor and a transplant recipient indicates a higher probability that this mismatched residue might be recognized by components of the immune system (antibodies, B-cells, T-cells etc.), as it constitutes a potential B-cell and/or antibody epitope.

The data presented in the examples herein shows a strong correlation between the present method (Snowball) and the data acquired by the prior art method “ElliPro” (FIG. 5). However, specifically in the characteristic helix structures of the HLA binding groove, the present method (‘Snowball’) is more sensitive than the prior art ElliPro approach, allowing to better distinguish inward facing amino acids and outbound amino acids (FIG. 6). The characteristic shape of HLA proteins is thus better described by repeated local ellipsoid fitting than by global ellipsoid fitting. Furthermore, the applied method is also applicable to other proteins than HLA. In particular for proteins folding into non-elliptic structures, this method may be beneficial in protrusion estimation. An advantage of the Snowball approach is its configurability to the size of the corresponding paratope, which may differ depending on the specific target receptor.

In embodiments, the method therefore relates to a computer-implemented method for determining an amino acid residue protruding from the surface of a human leukocyte antigen (HLA) protein structure, comprising repeated ellipsoid fitting for multiple amino acid atoms of the protein (structure), otherwise termed repeated local ellipsoid fitting.

Considering the larger structure by local ellipsoid protrusion adds value to solvent accessibility, that may be predicted high in overhanging regions concealing the amino acid in question yet being inaccessible for considerably large antibody recognition sites. Also, predicting surface area as a metric for solvent accessibility is not compensating for the specific size of an amino acid. Consequently, the small Glycine will on average have a smaller surface area compared to large amino acids like Tryptophan. Increased probe sizes and amino acid size normalization may thus be improvements for the prior art prediction pipeline.

Opposed to that, the method according to the invention (“also termed herein Snowball”) is amino acid size independent.

Another advantage of the present method for determining the protrusion of an amino acid residue over the prior art approach of determining the solvent surface accessibility, is that such prior art methods are not able to distinguish if a solvent accessible residue is actually protruding from the surface, namely being actually accessible to e.g., components of the immune system such as B-cells or antibodies, or if a solvent accessible residue is actually aligned with the surface of a protein, such that it is not necessarily recognized by e.g., components of the immune system, but only present on the surface but aligned to it (lies on the surface but does not protrude from). The present method, however, facilitates to determine the actual accessibility of a residue, such as an amino acid residue of a protein mismatched between a donor and transplant recipient, by determining, if the residue is actually protruding from the surface.

In a preferred embodiment of the method according to the invention step (i) comprises fitting an ellipsoid to a subset of atoms of the HLA protein structure that are in spatial proximity to each other, wherein the ellipsoid is centered to one atom of said subset of atoms, and wherein the ellipsoid comprises (or spans/covers/considers) atoms surrounding the atom at the center of the ellipsoid in a radius of 1-100 Å (angstrom) depending on the paratope size, preferably in a radius of 15 Å (angstrom) when e.g., considering human antibodies.

In embodiments the ellipsoid comprises (or spans/covers/considers) atoms surrounding the atom at the center of the ellipsoid in a radius of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 60, 70, 80, 90, 100, 150, 200, 250, 300, 350 Å (angstrom) or between 1-300, 1-200, 1-100, 100-300, 1-50, 1-40, 1-30, 1-35, 9-35, 1-20, 10-20, 1-15, 20-30, or of ≤300, ≤200, ≤100, ≤50, ≤35, or ≤15 Å (angstrom), and in some embodiments ≤100 Å or between 1-100, 1-40, 10-20, 9-35 or 15 Å.

In embodiments the ellipsoid comprises (or spans/covers/considers) atoms surrounding the atom at the center of the ellipsoid in a radius of 15 Å (angstrom), relating to an area of a circle of ~700 Å², for example corresponding to the antigen binding site of human HLA antibodies.

In embodiments the present method iterates over atoms of HLA protein structure data, thereby fitting ellipsoids to substructures (subsets) of atoms in proximity to the current center atom (e.g., with a radius between 1-100 Å, or specifically a radius of 15 Å). In embodiments the atoms' protrusion is ranked based on the ellipsoid's axes with the furthest atom being ranked 1.0 and the atom centered in the ellipsoid being ranked 0.0. In embodiments the median of all structure's atoms' protrusion ranks is formed and for each residue the maximum of its atomic protrusion medians is considered the corresponding amino acid's protrusion rank.

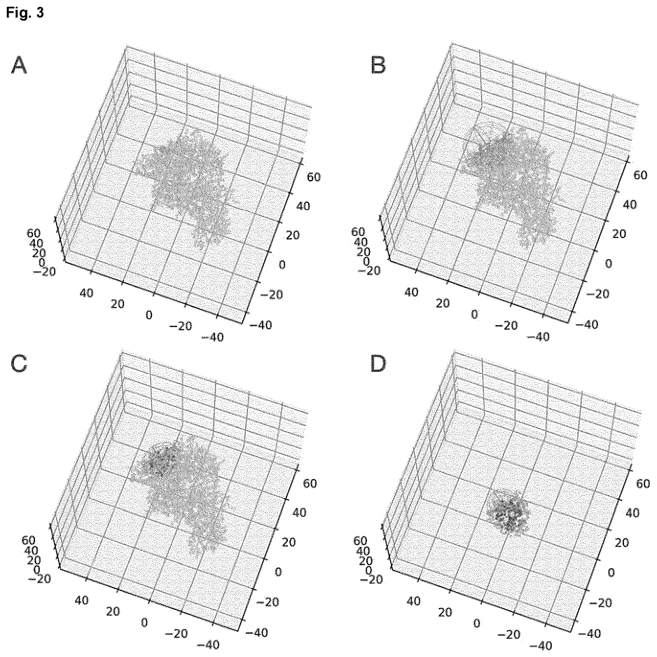

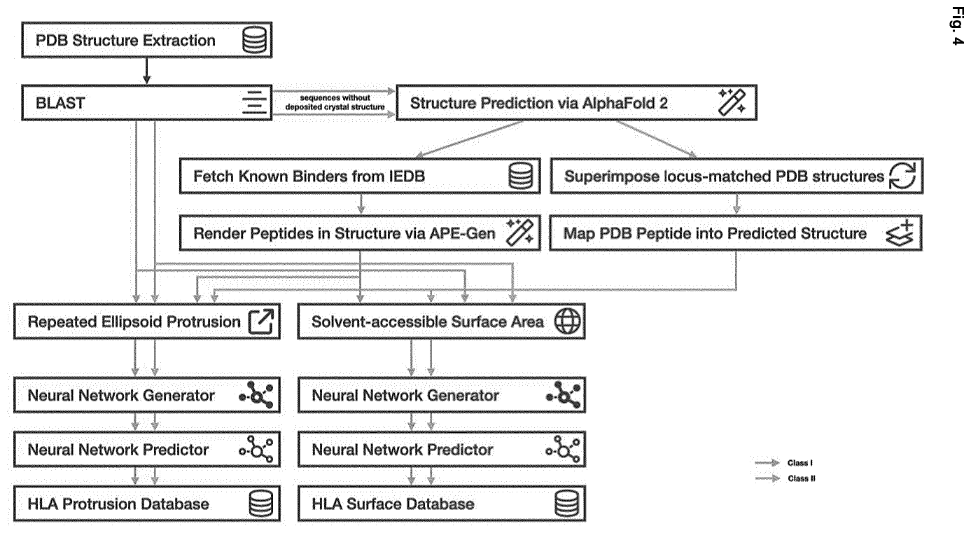

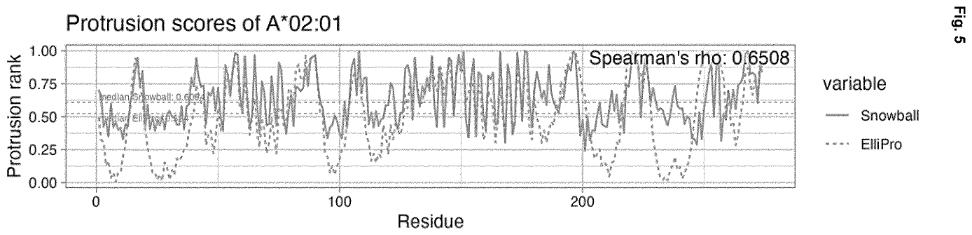

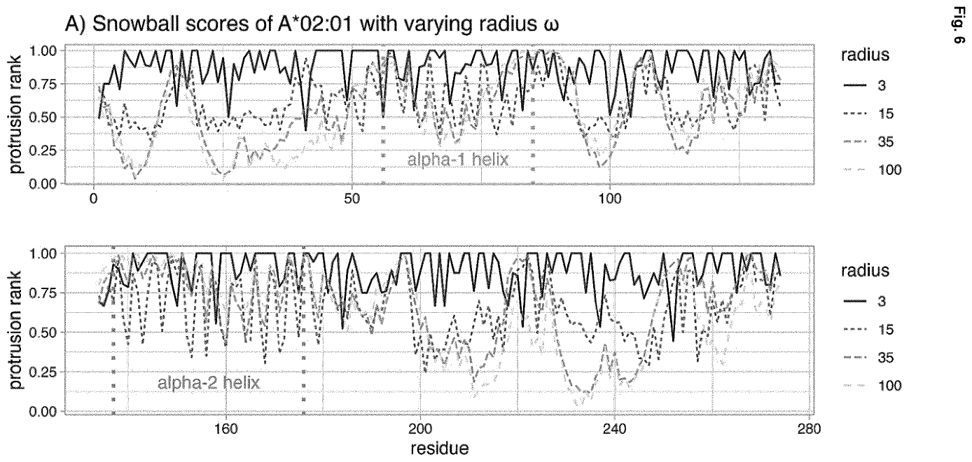

In embodiments the present method iterates over atoms of a HLA protein structure, e.g., as the respective center, wherein preferably multiple or all nearby atoms within an Euclidean distance w are selected. In embodiments, for said atoms, preferably after regularization, an ellipsoid is fitted and/or geometrically centered considering linear least squares. In embodiments, the distance between sphere center and transformed atom coordinate is ranked, preferably corresponding to the protrusion of fitted ellipsoid(s). In embodiments, for multiple or each atom(s) of a given structure, median protrusion ranks within all fitted ellipsoids may be calculated. In embodiments, the maximum median protrusion rank of multiple or each atom(s) of a residue may be considered as residue-specific protrusion rank (‘Snowball score’) per structure, e.g., per HLA protein. A non-limiting exemplary embodiment is described in Example 1 and FIG. 3.

In a preferred embodiment of the method according to the invention in step (i) an ellipsoid-fitting algorithm is employed to a subset of atoms of an HLA protein structure that are in spatial proximity to each other, wherein an ellipsoid is centered to an atom of the HLA protein structure.

In embodiments the spatial proximity of atoms to each other within a subset of atoms refers to a (spatial) distance between the atom at the (spatial) center of the subset of atoms to an atom of the subset that is farthest (has the greatest (spatial) distance from the center of the subset) from the center of the subset of 1-100 Å (angstrom). In embodiments the spatial proximity of atoms to each other within a subset of atoms refers to a group of atoms that are not further apart from each other than 1-300 Å (angstrom). In embodiments the spatial proximity of atoms to each other may refer to a group or subset of atoms that may be spanned by/fitted into an ellipsoid with a radius of 1-100 Å (angstrom).

In embodiments the spatial proximity of atoms to each other refers to a subset of atoms to which an ellipsoid may be fitted in step (i) wherein one atom of the subset constitutes the center of the fitted ellipsoid and the ellipsoid spans surrounding atoms (of the atom at the center) in a radius of 1-100 Å (angstrom).

It was entirely surprising that applying repeated local protrusion analysis of atoms of a protein and/or amino acid residue according to the present method, comprising repetitively fitting ellipsoids to a subset of atoms of said protein and/or amino acid residue, is able to predict residue protrusion with an even better reliability and sensitivity as prior art methods, e.g., ElliPro.

Importantly, the repeated local protrusion analysis according to the invention, e.g., as described in the claims and examples herein, facilitates a more precise determination of residue protrusion than prior art methods. For example, FIG. 5 depicts the results of such an application of the present method in comparison with the prior art method ElliPro, which is further described in Example 1.

As shown in FIG. 5, repeated local protrusion strongly correlates with the ElliPro score, but the mean protrusion rank determined by the present method is however higher than average ElliPro scores (invention=“Snowball”, dark grey curve on top of ElliPro curve). FIG. 6 illustrates that varying sphere radii ω (3-100 angstrom) indicate the sensitivity of the method according to the invention towards small structural changes. It can be observed that with large ω, the calculated protrusion ranks converge to calculations of ElliPro. Conversely, very small ω correspond to almost constantly high protrusion ranks. Interestingly, the suggested w of 15 Å surprisingly even allows reliable discrimination of amino acids' protrusion in the HLA helices (FIG. 6). The inventors surprisingly found that sphere radii impact sensitivity of repeated ellipsoid fitting on local structural changes, as per the heatmap of protrusion demonstrated below. Therefore, small radii (e.g., 3 Å) are susceptible to overfitting, whereas large radii consider solvent-inaccessible residues as protruding compared to the overall structure (e.g., 50 Å or 100 Å), similar to ElliPro's global protrusion ranks (100 Å). Therefore, specifically in the characteristic helix structures of the HLA binding groove, the present method is more sensitive and specific than ElliPro, allowing to better distinguish inward facing amino acids and outbound amino acids (FIG. 6). The characteristic shape of HLA proteins is thus better described by repeated local ellipsoid fitting according to the invention than by global ellipsoids, as used in the prior art. In particular, for proteins folding into non-elliptic structures, the present method is beneficial in protrusion estimation.

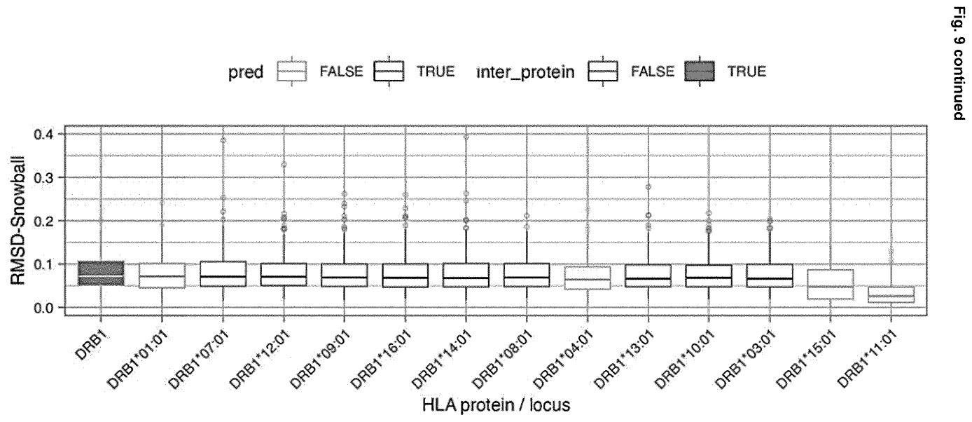

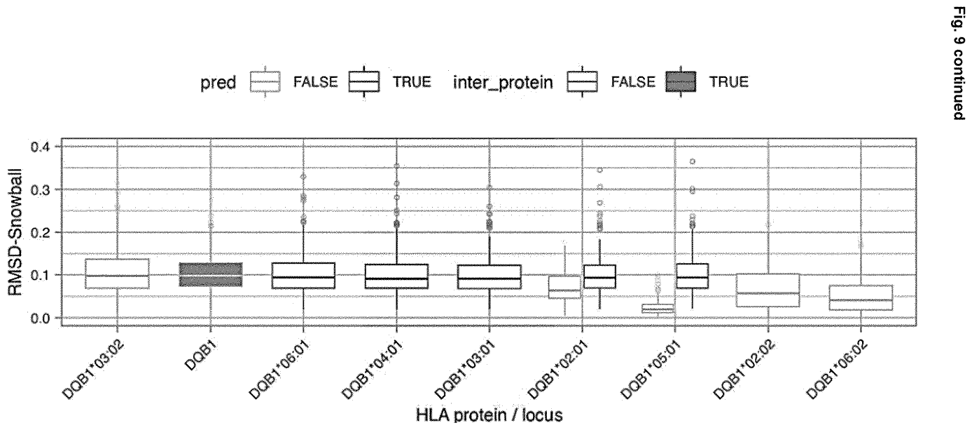

The method according to the invention enables a superior prediction of residue surface protrusion than prediction of solvent accessible residues, as shown in Example 1. Across all analyzed HLA loci, the present method's mean and median scores were higher than respective scores of the prior art method for prediction of solvent accessible residues (“Snowflake”). However, a strong correlation between the two methods Snowball and Snowflake can be observed with Spearman's rho above 0.8 (see FIG. 8). In addition, opposite to the Snowflake method, the present method is amino acid size-independent, which constitutes a significant improvement and compensates for unspecific signal using the Snowflake method.

In summary, the repeated local protrusion analysis comprising the repeated fitting of ellipsoids to a subset of atoms of an amino acid residue according to the invention facilitates a higher precision of the prediction of residue protrusion and an increased reliability of the prediction when compared to prior art methods. The herein disclosed method constitutes an improved method for identifying amino acid protrusion of proteins, such as HLA molecules, to support the definition of antibody accessibility in HLA epitope definition. The combination of the present method with the prior art method Snowflake (leading to “Snow”), which combines surface area and repeated ellipsoid protrusion as two markers of accessibility, has the surprising and advantageous potential to increase specificity of amino acid mismatch score approaches in HLA matching for transplantation.

In a preferred embodiment the invention relates to a computer-implemented method for determining an amino acid residue protruding from the surface of a human leukocyte antigen (HLA) protein structure, comprising:
(i) employing an ellipsoid-fitting algorithm to an atom of an HLA protein structure, and
(ii) repeating step (i) for multiple atoms of the HLA protein structure, preferably for each atom of the HLA protein structure, thereby creating multiple fitted ellipsoids, and
(iii) determining whether an amino acid of an HLA protein structure is surface protruding by considering the spatial distribution of atoms of any given amino acid residue relative to the position of the multiple fitted ellipsoids.

In an embodiment of the method of the invention step i) comprises fitting an ellipsoid to an atom of the HLA protein structure, wherein the atom constitutes the center of the fitted ellipsoid and the ellipsoid spans surrounding atoms in a radius of 1-100 Å (angstrom).

In an embodiment of the method according to the invention the step (i) further comprises calculating, within a fitted ellipsoid, a protrusion rank for multiple atoms, preferably for each atom, of the HLA protein structure, wherein the protrusion rank is determined based on the distance of a respective atom from the center of the ellipsoid considering its axes lengths, preferably wherein the atom furthest from the center is assigned a protrusion rank of 1 and the atom at the center of the ellipsoid is assigned a protrusion rank of 0.

In an embodiment of the method according to the invention step (iii) comprises determining whether an amino acid of an HLA protein structure is surface protruding by considering the ellipsoid protrusion ranks of atoms of any given amino acid residue relative to the position of the multiple fitted ellipsoids.

In an embodiment the method according to the invention comprises:
calculating, for each atom of the HLA protein structure, a median value of the protrusion ranks (protrusion rank median) determined in steps (i)-(ii) for the respective atom, and
determining for multiple, preferably for each, amino acid residues of the HLA protein structure a protrusion rank (amino acid protrusion rank), wherein the greatest protrusion rank median of the amino acid's atoms is considered the amino acid protrusion rank.

In an embodiment of the method according to the invention the HLA protein structure and the surface protruding residues comprise (i) an extracellular alpha chain, a beta-2-microglobulin and an HLA-bound peptide structure, in case of HLA Class I proteins, or (ii) the protein's extracellular alpha and beta chains, and an HLA-bound peptide structure, in case of HLA Class II proteins.

The consideration of complete HLA structures in embodiments, including e.g., alpha and beta chains, with the HLA-bound peptide, appears to represent an improvement over earlier approaches, and enables a more accurate determination of which residues of the HLA protein sequence are in fact surface protruding and accessible to immune cells and thus a potential B cell epitope target.

Rendering peptides of experimental Class II structures into superimposed predicted structures is a simplistic approach which assumes similar peptide alignments across different Class II protein binding grooves. Including a more robust approach to predict Class II peptide docking improves downstream predictions.

The herein-mentioned algorithms for determining surface protruding residues are functional and, preferably in combination with the peptide-bound HLA structures, appear to lead to improved prediction of surface protrusion. Additional means and algorithms for determining surface protruding residues are known to a skilled person and may be employed accordingly.

In another aspect the present invention relates to a computer-implemented method for producing a database of predicted surface-protruding residues for multiple human leukocyte antigen (HLA) proteins, comprising:
a. Providing one or more HLA protein amino acid sequences, for which a protein structure has been experimentally determined,
b. Predicting HLA protein structure of one or more HLA proteins, for which a structure has not been experimentally determined,
c. Supplementing the predicted HLA protein structures of b. with an HLA-bound peptide, if the structure of b. does not exhibit an HLA-bound peptide,
d. Determining surface-protruding residues of each HLA protein structure of a. and c. using the method according to the invention for determining an amino acid residue protruding from the surface of a human leukocyte antigen (HLA) protein structure,
e. Generating a database of surface protruding residues for the HLA proteins of a. and c. and HLA proteins additional to those of a. to c., comprising employing a neural network trained on amino acids sequences of the HLA proteins of a. and c. and the corresponding surface-protruding residues determined in d.

In embodiments of the computer-implemented method for producing a database according to the invention, the neural network according to e) comprises a long short-term memory bidirectional recurrent neural network.

This constellation of neural network type, and in- and output information, leads to a beneficial training set with which surface protrusion may be determined with greater accuracy than earlier approaches.

In embodiments the neural network considers the provided structure(s) of HLA protein(s), the linear amino acid sequence of the HLA protein(s), the amino acid sequence of a peptide (preferably 9 amino acids in length) bound (presented by) to the HLA protein(s) and/or the surface protrusion of an amino acid residue and/or the solvent accessibility of an amino acid residue that is determined according to the invention, preferably to predict the accessibility to the solvent and/or immune system components of at least one certain amino acid mismatch between a donor and a transplant recipient.

As described in greater detail herein, the inventors have developed a deep learning prediction pipeline for surface-protrusion defining allele-specific protruding epitopes. The protrusion prediction methodology described in the examples, and in embodiments of the invention, is trained on experimental HLA structures of the Protein Data Bank (www.rscb.org), supplemented by HLA structures predicted by Deepmind's recent AlphaFold protein folding predictor. [34-35].

Limiting on protrusion as predicted output, the present invention's deep learning model is preferably trained on HLA experimental structures, and optionally complemented by predicted HLA structures.

One embodiment of the present method is depicted in FIG. 4 as a schematic workflow.

Previous B cell epitope matching approaches considered HLA protein structures of different alleles of the same locus or Class being conserved despite the alleles' heterogeneity. The present invention unexpectedly found allele-specific differences in the surface protrusion of amino acid residues that suggest considering the surface on an individual basis rather than assuming identical structure.

The present invention provides advantages over earlier methods of the prior art. For example, the HLA-EMMA algorithm appears to be disadvantageous by limitation to an analysis of experimentally determined HLA structures, as solvent accessible polymorphic amino acid positions were determined using only publicly available crystal structures and solvent accessibility prediction tools. However, no mention is made of additionally using predicted HLA structures to complement and expand the analysis.

The HLA-EMMA algorithm also appears to not use structures supplemented with HLA-bound peptides, thus reducing the practical accuracy and applicability of the method.

Furthermore, the HLA-EMMA method appears not to employ artificial intelligence or machine learning to predict solvent accessible surface residues based on HLA sequence alone, after being trained on HLA sequences, structures and predicted solvent accessible surface residues. HLA-EMMA appears to only use experimentally established structures to assess solvent accessible surface residues, and is thus limited to those HLA proteins for which structures have been solved. In contrast, the method of the present invention enables database construction of surface protruding residues for HLA proteins for which no structure has been experimentally solved or predicted. By way of using a neural network, trained on HLA sequences, structures and predicted surface protruding residues, analysis of further HLA sequences for which no structure is known or predicted may be analysed and surface protruding residues for such HLA proteins may be predicted.

The production of such a database is of immediate relevance for computational and health benefits. Such a database can be subsequently used in assessing or predicting risk of potential unwanted immune reactions against mismatched HLA protein sequences and potential surface protruding and accessible mismatched residues. In future analysis of predicted risk in transplantation scenarios or other scenarios where B cell epitope prediction may play a role, the database can be consulted with and not only HLA mismatches between e.g., donor and recipient interrogated, but also the surface protrusion of the mismatched residue may be considered, when considering whether any given mismatch is likely to lead to a relevant B cell epitope that could cause an unwanted immune reaction. Thus, the generation of the database itself is of inherent medical benefit.

Computational benefit is also evident, as the method leads to a database that avoids having to solve or predict HLA structures in order to assess surface protrusion and accessibility for any given residue. The use of a neural network, trained as described herein, thus enables generation of a database that avoids computationally intensive analysis based on HLA structures to determine surface protrusion and accessibility of amino acid residues. Previous approaches required structural data of the HLA protein, which was then analysed to assess which residues were surface protruding. In embodiments, the present invention thus avoids such computationally intensive approaches, and enables prediction of surface protrusion using a trained neural network, based on HLA sequence alone as input, allowing real-time application.

As described in more detail herein, a suitable neural network may be elected for the particular use based on knowledge of a skilled person. The training of a neural network on HLA amino acid sequences and corresponding surface protruding residues as determined for any given example HLA proteins, represents a novel and inventive step in improving means to generate a database of surface protruding residues for the HLA proteins.

In other aspects and embodiments, the invention therefore relates to a method for training a neural network on HLA amino acid sequences and corresponding surface protruding residues, as may be determined for any given example HLA proteins, in order to improve prediction surface protruding residues for further previously unanalyzed HLA protein sequences.

In embodiments, the input and output of the neural network may be configured accordingly.

In one embodiment, the input for the neural network according to step e) comprises the HLA alpha chain (for HLA Class I) and/or the HLA alpha and beta chains (for HLA Class II) amino acid sequence(s).

In one embodiment, residue-specific information on surface protrusion is an output.

In embodiments of the invention, the invention relates to a database, and methods of generating the same, the database comprising surface protruding residues for the HLA proteins of a. and c. and HLA proteins additional to those of a. to c., comprising employing a neural network trained on amino acids sequences of the HLA proteins of a. and c. and the corresponding surface protruding residues determined in d.

In embodiments, the HLA proteins of a. are any given HLA protein for which a structure has been experimentally determined. Such HLA proteins may be identified by a skilled person without undue effort. Various databases are publicly available where HLA structures are presented, information is typically provided in such databases on the experimental approach used and the resulting structure that was obtained and analyzed.

In embodiments, the HLA proteins of b. are any given HLA protein for which a structure has not been experimentally determined. In embodiments, the HLA proteins of b. are any given HLA protein for which an experimentally determined HLA structure is not employed in the present method. Such HLA sequences are commonly available to a skilled person, the HLA proteins of b. are preferably any HLA protein not yet structurally solved, or an HLA protein where any experimental structure is simply not employed in the method, and the structure of such an HLA protein is thus predicted using (preferably pre-established) HLA structure prediction methods.

In embodiments, the HLA proteins of c. include an HLA-bound peptide, supplemented to the predicted structure of b.

In embodiments, “HLA proteins additional to those of a. to c.” are in essence any given further HLA protein that has not been included in the earlier method steps. Databases are available to a skilled person providing many HLA sequences. HLA structures have not been prepared for all such sequences, and the present invention seeks to provide a database of predicted surface-protruding residues not only for HLA proteins based on established (a.) or predicted (b.) structures, but also based simply on any given HLA sequence.

Thus, in preferred embodiments of the present method, HLA proteins with experimentally determined structures (a.) and HLA proteins with predicted structures (b.) and predicted HLA structures supplemented with HLA-bound peptides (c.) are analyzed to determine surface protruding residues, using for example the particular methods disclosed herein. This information regarding surface protruding residues for proteins a., b. and c. is thus used to train a neural network, or other suitable artificial intelligence or machine learning algorithm, to surface protruding residues based on HLA sequence.

By doing so, the neural network is capable of “learning” how surface protruding residues correlate to HLA sequence and/or structure (experimentally determined (a.) or predicted (b/c.) and thereby is capable of processing any given HLA protein sequence, even those not assessed under a.-d., and predict surface protruding residues. These predicted residues may be presented in a database.

The generation of the database as described herein represents a novel approach towards predicting surface protruding residues for human leukocyte antigen (HLA) proteins. By identifying such surface protruding residues, potential B cell epitopes can be determined, thus indicating whether any given portion of an HLA sequence is presented on the surface of the intact molecule (protruding, and/or solvent accessible) and may therefore represent a target for antibody production.

Embodiments of the invention comprise comparing mismatched amino acids to a database of surface protruding residues for HLA proteins, wherein the surface protruding residues of the database are predicted by a neural network trained on amino acid sequences of experimentally determined and predicted HLA protein structures, and on the corresponding residues' surface protrusion.

By generating a database for multiple, preferably essentially all, HLA protein structures, the likelihood of whether any given mismatched HLA amino acid (mismatched for example between a donor and recipient of a transplantation) is predicted to be surface-protruding can be assessed. For example, once the database is established, and a potential HLA mismatch is determined between a donor and recipient, that mismatched amino acid can be compared to the database to determine whether it is predicted to be surface protruding, and hence surface accessible, in the context of the HLA structure. If surface protruding, the risk of the mismatch representing a B cell epitope and thus potentially an antibody target can be determined.

In embodiments of the computer-implemented method for producing a database according to the invention the supplementing according to c. comprises:
For predicted structures with known binding peptides, adding said known peptides to the HLA protein structure, and/or
for predicted structures without known binding peptides, adding peptides to the HLA protein structure that are known to bind to an HLA allele with a high degree (the greatest degree) of amino acid sequence identity in extracellular domains to the HLA amino acid sequence of said predicted structure without known binding peptides.

This approach therefore is carried out in consideration of HLA structures bound by peptide sequences known to bind the HLA protein (allele-specific), or peptides known to bind similar HLA proteins (based on HLA sequence similarity), thus an accurate combined representation of HLA structure, including both the HLA molecule itself and any peptide presented by said HLA, is assessed for potential surface protruding resides.

In embodiments of the computer-implemented method for producing a database according to the invention the neural network according to step e) comprises the HLA alpha chain (for HLA Class I) or the HLA alpha and beta chains (for HLA Class II) amino acid sequence(s) as an input and the surface protruding residues as an output.

In embodiments of the computer-implemented method for producing a database according to the invention the database according to step e) comprises surface protruding residues for essentially all HLA proteins (all HLA alleles) in any given HLA Class.

By generating such a database, an optimal reference data set is provided to which any given HLA mismatch can be compared, for example prior to transplantation, in order to determine a risk of a B cell/antibody-mediated immune reaction against said mismatch. It must be considered that any given learning-based surface prediction is obsolete if predicted structures have been generated for all known HLA alleles. However, structure prediction is time and computer-power costly, thus making it practically challenging, if not entirely implausible, to have to predict structural information for all HLA alleles. In order to present a complete or near complete database, the present invention, in embodiments, employs a subset of determined HLA structures, combined with a subset of in silico predicted HLA structures, and a prediction of accessible binding residues, to train a neural network capable of assessing other HLA alleles/sequences. Thus, the neural network of step e) is trained on this combination of data sets, and subsequently allows a machine learning process to determine the surface protruding residues for any number of additional HLA alleles based on their protein sequence.

In embodiments of the computer-implemented method for producing a database according to the invention the neural network according to step e) is HLA Class and/or HLA-locus specific, for example comprises multiple surface protruding residues for multiple human leukocyte antigen (HLA) proteins from only HLA Class I proteins, only HLA Class II proteins, or only for alleles selected from the same HLA locus.

Thus, a database can be generated as described herein, in which essentially all known HLA alleles of a given Class or HLA locus are assessed, and upon determining e.g., a mismatch between donor and recipient of transplantation material, the relevant database can be considered. This method enables a more efficient and/or quicker approach to interrogating relevant HLA mismatches, depending on the HLA Class of locus of interest.

In embodiments, the method is automated, preferably wherein the method does not comprise manual curation of the database according to verified antibody binding.

In contrast to other B cell epitope predictors known in the art, the present invention preferably does not rely on manual curation of the database based on known HLA epitopes, for example known epitopes to which antibodies have been detected. The absence of manual curation represents an improvement in automation in this approach and indicates that the means of the present invention are equally effective, if not more accurate, in predicting surface protruding and potential antibody-accessible, residues, compared to older approaches which require or employ manual curation of the database based on known epitopes bound by antibodies.

In embodiments of the computer-implemented method for producing a database according to the invention the method is automated, preferably wherein the method does not comprise manual curation of the database according to verified antibody binding.

A further embodiment of the invention therefore relates to a system for selecting and/or screening donor material for transplantation, for example for selecting donor material with permissible mismatches from HLA mismatched donors, comprising one or more software modules capable of carrying out one or more methods as described herein.

In one embodiment the method as described herein is characterized in that the information stored in the database is an electronically stored representation of biological data, or corresponds to, or is an abstraction of, one or more biological entities. The present invention therefore relates to a computer implemented method in which data are processed that correspond to “real-world” biological entities. The database as such is not only characterized by the means of its production, but preferably also by the particular biological context of the entities and relationships embodied in the database.

In one embodiment, the method as described herein is characterized in that HLA typing and/or matching between one or more subjects, for example donor and recipient of transplantation material, has been conducted in advance of carrying out the method described herein. The HLA-typing data of the method may have been carried out in advance, and the data regarding HLA-type subsequently analyzed via the methods of the present invention.

In one embodiment the HLA protein sequences relate preferably to an informational and/or computational representation of an HLA allele. In a preferred embodiment the HLA sequences relate to alleles that are defined by a unique protein sequence, whereby HLA alleles that show only DNA sequence differences without changes in the corresponding protein sequence are in some embodiments preferably not considered in the method. An example of such HLA alleles that show only DNA sequence differences relate to allele-specific features described in fields 3 and 4 of the standard HLA nomenclature system relating to synonymous DNA sequence changes or sequence changes outside a coding region.

In one embodiment the selected HLA molecules for assessment are the HLA loci A*, B*, C*, DRB1* and/or DQB1*, and optionally DQA1*, DPA1*, DPB1* and DRB3/4/5*.

A further aspect of the invention relates to a system for selecting and/or screening donor material for transplantation, for example for selecting donor material with permissible mismatches from mismatched donors, comprising software modules for determining B cell epitopes, as described herein. In one embodiment, the system for selecting and/or screening donor material for transplantation is characterized in that said HLA software module carries out HLA matching between donor and recipient HLA typing data on the basis of high-resolution allele coding (e.g. A*02:01), preferably for HLA loci A*, B*, C*, DRB1* and/or DQB1*, and optionally DQA1*, DPA1*, DPB1* and DRB3/4/5*.

In one embodiment, the system for selecting and/or screening donor material for transplantation is characterized in that said HLA software module carries out HLA epitope matching between donor and recipient HLA typing data on low or intermediate resolution data (i.e., ambiguous HLA typing). By multiple imputation of potential genotypes matching with the given low or intermediate resolution input data and weighting genotypes by haplotype frequencies, multiple epitope match results are generated for a single recipient donor pair. These results are aggregated together based on said haplotype frequencies. Consequently, the invention presented herein is capable of being used in settings, where high resolution HLA typing data is not available with current technologies, e.g., due to time constraints (e.g., deceased donor kidney allocation). Opposed to that, existing algorithms for B cell epitope matching require protein-level typing for both recipient and donor.

In some embodiments, the method comprises additionally incorporating the information generated by the methods of the present invention for multiple donors and/or multiple recipients, for example wherein the data outputted by the present invention is incorporated in a list of HLA mismatched donors and/or recipients with acceptably low or unacceptably high numbers of B cell epitopes, in the context of any given transplantation.

The various embodiments of the present invention provide approaches towards HLA matching providing a unique service to transplantation providers searching for transplantation material with low risk of causing unwanted allogenic reactions. The technical effect of the invention described herein is not only the improved speed of interrogation of donor databanks, but also the provision of safer transplantation material, thereby increasing/prolonging the health of the patient population having received transplantations of allogenic material.

In one embodiment, donor profiles are preferably stored in an additional data structure, database, and/or library. In one embodiment, multiple donor profiles or donor types are stored in a donor data structure, database, and/or library comprising preferably essentially all possible donors for essentially all possible genotypes or possible combinations of HLA alleles, and/or haplotypes.

The inventors recently proposed the “Snowflake” amino acid matching score. [8] In brief, Snowflake computes the predicted surface area of donor HLA proteins' amino acid positions. Amino acids exceeding a surface area threshold are considered as being exposed and compared to recipients' self HLA proteins. Exposed amino acids being mismatched with the patients increase the Snowflake score by one. It has been shown that surface area varies within HLA loci or Class which lead to the Snowflake model considering surface area protein-specifically. [8]

The authors herein suggest an improvement of said prior art method named “Snow”, which combines the “Snowflake” model with the method according to the invention described herein that applies repeated ellipsoid protrusion ranking of amino acid positions, named “Snowball”. The present method (Snowball) iterates over atoms of a protein structure, such as the HLA protein structure, fitting ellipsoids to substructures of atoms in proximity to a current center atom and ranking atoms' protrusion with respect to fitted ellipsoids.

Therefore, in embodiments, the present method may also be combined with or can comprise additionally determining one or more predicted solvent accessible surface residues of a human protein, such as a leukocyte antigen (HLA) protein (this embodiment being termed Snowflake).

In embodiments of the computer-implemented method for producing a database according to the invention further comprises additionally determining one or more predicted solvent accessible surface residues of a human leukocyte antigen (HLA) protein, wherein the predicted solvent-accessible surface residues and protruding amino acid residues of a human leukocyte antigen (HLA) protein indicate a human leukocyte antigen (HLA) B-cell epitope.

The Snowflake predictor for HLA surface area prediction is comparable to both NetSurfP 2.0 and PaleAle 4.0 in their neural network architecture. These predictors implement a deep learning architecture using BRNN to predict (amongst other characteristics) relative surface accessibility based on surface area. Both networks have been trained with the experimental structures reported in the PDB (Klausen M S, et al. NetSurfP-2.0: Improved prediction of protein structural features by integrated deep learning. Proteins. 2019 June; 87(6):520-7; Mirabello C, Pollastri G. Porter, PaleAle 4.0: high-accuracy prediction of protein secondary structure and relative solvent accessibility. Bioinformatics. 2013, 15; 29(16):2056-8). The training data used to train the Snowflake surface area predictor however is specialized for HLA molecules and experimental structures are supplemented by predictions of dedicated protein folding predictors (e.g., Alphafold), aggregating more domain-specific data. In embodiments, these simulated HLA structures are also considered by the Snowball predictor for estimation of local repeated amino acid protrusion. In embodiments of the invention, the methods, embodiments or other aspects of the present invention are combined with embodiments of the Snowflake predictor, thus joining both analyses into the Snow algorithm, suitable for predicting antibody accessibility.

In another aspect the present invention relates to a computer-implemented method for predicting a human leukocyte antigen (HLA) B-cell epitope that is mismatched between two or more subjects, comprising:
i. Determining one or more mismatched amino acids of an HLA protein between two or more subjects,
   comprising preferably identifying amino acid similarity of an HLA protein of one subject with the same amino acid positions in one or more HLA proteins of another subject,
ii. Determining whether said mismatched amino acids are predicted to be protruding from a surface of an HLA structure, preferably according to the methods described herein,
   comprising preferably comparing said mismatched amino acids to a database of surface-protruding residues for HLA proteins, wherein the surface-protruding residues of the database are predicted by a neural network trained on amino acids sequences of experimentally determined and predicted HLA protein structures, and on the corresponding residues' surface protrusion,
iii. wherein a mismatched amino acid predicted to be surface-protruding indicates a human leukocyte antigen (HLA) B-cell/antibody epitope that is mismatched between two or more subjects.

Through this method, one can ascertain whether any given mismatched HLA amino acid, for example between a donor and recipient of transplantation material, is predicted to be surface-protruding and therefore a potential B cell/antibody epitope. In embodiments, this aspect of the invention is also defined by the surface-protruding residues of the database being predicted by a neural network trained on amino acid sequences of experimentally determined and predicted HLA protein structures, and on the corresponding residues' surface protrusion.

The advantages of the database of the invention, as described above, are also applicable to the method of the invention for predicting a human leukocyte antigen (HLA) B-cell epitope that is mismatched between two or more subjects.

In embodiments, the computer-implemented method for predicting an HLA B-cell epitope that is mismatched between two or more subjects produces a score. The score reflects the number of allele-specific surface-protruding amino acid mismatches identified between two subjects.

In one embodiment, the method of the invention for predicting a human leukocyte antigen (HLA) B-cell epitope that is mismatched between two or more subjects, is characterized in that the database is produced using a method for producing a database of predicted surface protruding residues for multiple human leukocyte antigen (HLA) proteins, as described above.

In one embodiment, a mismatched amino acid predicted to be surface protruding indicates an antibody immune response against the corresponding mismatched HLA protein.

The method as described herein therefore also provides an indication of whether a mismatched HLA amino acid is immunogenic, and thus potentially a risk factor in transplantation of material between HLA mismatched subjects.

In embodiments of the computer-implemented method for predicting a mismatched human leukocyte antigen (HLA) B-cell epitope the database is produced using a method as described herein.

In embodiments of the computer-implemented method for predicting a mismatched human leukocyte antigen (HLA) B-cell epitope a mismatched amino acid predicted to be surface protruding indicates an antibody immune response against the corresponding mismatched HLA protein.

In embodiments of the computer-implemented method for predicting a mismatched human leukocyte antigen (HLA) B-cell epitope comprises additionally determining one or more predicted residues with high solvent-accessible surface area of a human leukocyte antigen (HLA) protein, wherein a mismatched amino acid predicted to be surface-protruding and with high solvent-accessible surface area indicates an antibody immune response against the corresponding mismatched HLA protein.

In another aspect the present invention relates to a computer-implemented method for selecting transplantation material for allogeneic transplantation between subjects with one or more human leukocyte antigen (HLA) mismatches, comprising assessing whether one or more human leukocyte antigen (HLA) B-cell epitopes are mismatched between said subjects according to the method for predicting a human leukocyte antigen (HLA) B-cell epitope that is mismatched between two or more subjects, wherein the number of determined mismatched HLA B-cell epitopes positively correlates with an antibody immune response against a corresponding mismatched HLA protein and/or a likelihood of an adverse immune reaction after transplantation.

In embodiments of the computer-implemented method for selecting transplantation material for allogeneic transplantation the transplantation comprises solid organ transplantation (such as kidney transplantation, liver transplantation, heart transplantation, or lung transplantation), transplantation of stem cells (such as transplantation of hematopoietic stem-cells (HSCT), cord blood, cord blood cells or induced pluripotent stem cells), or transfusion of blood or cell products (such as platelet transfusion, or engineered T cells).

The data presented in Example 2 confirms that, when the prior art PIRCHE model, which aims on predicting the indirect pathway, is considered next to the present Snow method, optimal Snow thresholds shift it towards being a specialized predictor for the antibody pathway, in conjunction outperforming predictive performance of simple amino acid matching, which thus can be considered as a generalist predictor for immune response. This proves the additive value of considering multiple specialized prediction models for the different pathways of allorecognition simultaneously and supports the theory of linked recognition in transplant immunology.

In summary, in Example 2 herein, the presented evidence shows that the Snow method according to the invention, i.e., amino acid matching based on surface area and protrusion, is a powerful predictor of kidney graft survival. Also, the data proves optimal Snow thresholds that outperform amino acid mismatch scores in conjunction with HLA-A-B-DR matching and prior art PIRCHE matching. This supports the hypothesis of linked recognition and the importance of modeling and considering individual pathways of allorecognition simultaneously.

In further embodiments the present method may also be combined with or can comprise additionally determining the number of predicted indirectly recognized HLA epitopes (PIRCHEs).

In embodiments of the computer-implemented method for selecting transplantation material for allogeneic transplantation comprises additionally determining one or more mismatched HLA T-cell epitopes between said subjects, preferably comprising determining the number of predicted indirectly recognized HLA epitopes (PIRCHEs), wherein said PIRCHEs are recipient- or donor-specific HLA-derived peptides from a mismatched recipient or donor HLA allele, respectively, and are predicted to be presented by an HLA molecule, wherein the number of determined mismatched HLA T-cell epitopes positively correlates with a likelihood of an adverse immune reaction after transplantation.

In embodiments, donor profiles are preferably stored in an additional data structure, database, and/or libraries. In embodiments, multiple donor profiles or donor types are stored in a donor data structure, database, and/or library comprising preferably essentially all possible donors for essentially all possible genotypes or possible combinations of HLA alleles, and/or haplotypes.

The phrase “essentially all” relates in this context to a significant number of, preferably all, or every known, protein variant of the allele group. The method therefore can effectively determine whether a particular donor or allele group indeed is associated with a relatively low number of predicted B cell epitopes and therefore present itself as a preferred selection.

Because those skilled in the art are able to make a pre-selection of possible protein variants within any given allele group, either on the basis of the reliability of the stored data or on population frequencies of certain alleles, the method is not limited to analysis of all known protein sequences of an allele group. In one embodiment the method will encompass the analysis of essentially all known (or relevant) protein sequences within the allele group, in order to provide more definitive results.

In one embodiment, the invention enables searching with recipient information (corresponding preferably to a biological recipient in need of a transplantation) against one or more donor profiles, wherein the result (outcome) of the present invention provides information on the one or more (preferably multiple) best-matched mismatched donor profiles, preferably a mismatched donor with a lowest possible number of determined (predicted) B cell epitopes. In one embodiment the invention is characterized in that the method and/or system is functionally connected to electronic registers of donor or patient material, wherein the outcome of the method and/or system of the invention enables a query to be sent from an end-user of said method and/or system to said register for any given one or more best-matched mismatched donor material corresponding to said outcome. The invention preferably links the end-user, for example a medical practitioner or transplant provider, with the stem cell, organ or other donor material registers that comprise information on said donor material. The transplantation material may relate to any given biological matter intended for transplantation. Such biological matter may relate to cells, organs, tissue, blood, in particular cord blood, hematopoietic stem cells, or other progenitor or stem cell populations, or organs for transplantation. Preferred relevant transplantation fields are solid organ transplantation (such as kidney transplantation, liver transplantation, heart transplantation, lung transplantation), transplantation of stem cells (such as transplantation of hematopoietic stem-cells (HSCT), cord blood, cord blood cells or induced pluripotent stem cells), or transfusion of blood or cell products (such as platelet transfusion or engineered T cells).

The computer implementation of the invention enables an efficient, fast and reliable method for identification of potentially permissible donor material for allogeneic transplantation. The data processed by the software can be handled in a completely or partially automatic manner, thereby enabling in embodiments an automated computer-implemented method. Data regarding the HLA typing of donor and recipient, in addition to the number of B cell epitopes determined for any donor-recipient pair, can be stored electronically and eventually maintained in appropriate databases. The invention therefore also relates to computer software capable of carrying out the method as described herein.

In one embodiment, the system of the invention preferably comprises one or more databases with information on all published HLA alleles. The database may be updated as new HLA allele sequences are published. Computer software, which could also be based on any given computing language, can be used for generating and/or updating the databases, in addition to calling the respective programs required.

In embodiments, the invention further comprises a method and/or system for preferably pre-transplantation prediction of an immune response against human leukocyte antigens (HLA), which may occur after transplantation. The system may comprise computing devices, data storage devices and/or appropriate software, for example individual software modules, which interact with each other to carry out the method as described herein.

In one embodiment the present invention relates to a method as described herein, wherein HLA typing is obtained for and/or carried out for HLA subtypes HLA-A, HLA-B, HLA-C, HLA-DRB1, HLA-DQB1, HLA-DPB1, -DQA1, -DPA1, -G, -E, -F, MICA, MICB and/or KIR. In one embodiment the present invention relates to a method as described herein, wherein HLA typing is provided and/or carried out on HLA-A, -B, -C, -DRB1 and -DQB1. In the domain of stem cell transplantation, these 5 alleles provide the basis for the commonly used terminology “9/10-matched”, which refers to a 9/10-matched unrelated donor. In such a case 9 of the 10 alleles of these 5 genes show a match but one remaining allele does not match. The present invention therefore allows, in one embodiment, determination of whether the use of material from such a 9/10-matched unrelated donor is safe, e.g., whether the mismatch is permissible for transplantation. In the domain of solid organ transplantation, sometimes only a subset of the named HLA subtypes (e.g., HLA-A, HLA-B and HLA-DRB1) is focused on.

The invention relates also to the method as described herein for finding permissible HLA-mismatches in donor samples where more mismatches are present than the common 9/10 scenario. One example of potential donors who are encompassed by the present invention are Haploidentical donors, who may be screened using the method described herein for permissible mismatched donor material. A haploidentical related donor, may be described as a donor who has a “50% match” to the patient. This type of donor can be a parent, sibling, or child. By definition, a parent or child of a patient will always be a haploidentical donor since half of the genetic material comes from each parent. There is a 50% chance that a sibling will be a haploidentical donor. Haploidentical Hematopoietic stem-cell transplantation (HSCT) offers many more people the option of Hematopoietic cell transplantation (HCT) as 90% of patients have a haploidentical family member. Other advantages include: immediate donor availability; equivalent access for all patients regardless of ethnic background; ability to select between multiple donors; and ability to obtain additional cells if needed. Alternatively, cord blood units (CBUs) may not show a high level of HLA-matching, but still be suitable for transplantation if the mismatches are permissible as determined by the method as described herein. CBU's are typically minimally 4/6 matched, but this match can however lead to a 4/10 or 5/10 situation at the allelic level.

In one embodiment the present invention relates to a method as described herein, wherein HLA typing comprises serological and/or molecular typing. In a preferred embodiment the present invention relates to a method as described herein, wherein HLA typing is carried out at high resolution level with amino acid sequence-based typing.

In one embodiment the present invention relates to a method as described herein, wherein HLA typing comprises sequencing of exon 1-7 for HLA Class I alleles and exon 1-6 for HLA Class II alleles. In one embodiment the present invention relates to a method as described herein, wherein HLA typing comprises high resolution HLA-A, -B, -C, -DRB1 and -DQB1 typing of exons 2 and 3 for HLA Class-I alleles and exon 2 for HLA Class-II alleles. These particular HLA-typing approaches are described in the examples provided herein and demonstrate advantages over earlier typing methods, providing full coverage of the alleles that need to be typed to identify the mismatches.

The various embodiments of the present invention provide approaches towards HLA matching providing a unique service to transplantation providers searching for transplantation material with low risk of causing unwanted allogenic reactions. The technical effect of the invention described herein is not only the improved speed of interrogation of donor databanks, but also the provision of safer transplantation material, thereby increasing and prolonging the health of the patient population having received transplantations of allogenic material.

Any embodiment of the invention disclosed in the context of the method, system or computer product, is considered disclosed in combination with the other related aspects of the invention. For example, the features of the method apply to the system, and vice versa. The embodiments or features of any given aspect may also be considered in combination with the features or embodiments of another aspect of the invention.

### DETAILED DESCRIPTION OF THE INVENTION

All cited documents of the patent and non-patent literature are hereby incorporated by reference in their entirety.

The present invention is directed to a computer-implemented method for determining an amino acid residue protruding from the surface of a human leukocyte antigen (HLA) protein structure. In embodiment, the method comprises: (i) employing an ellipsoid-fitting algorithm to a subset of atoms of an HLA protein structure, that are in spatial proximity to each other, (ii) repeating step (i) for multiple subsets of atoms of the HLA protein structure thereby creating multiple fitted ellipsoids, and (iii) determining whether an amino acid of an HLA protein structure is surface-protruding by considering the spatial distribution of atoms of any given amino acid residue relative to the position of the multiple fitted ellipsoids.

The term “subset of atoms” refers to a group, set or subset of atoms within a molecule, such as e.g., a protein. In some embodiments the subset of atoms refers to a group or set of one or more or at least two atoms that are in spatial proximity to each other.

An “ellipsoid” is a surface that may be obtained from a sphere by deforming it by means of directional scaling, or more generally, of an affine transformation. An ellipsoid commonly has three pairwise perpendicular axes of symmetry which intersect at a center of symmetry, called the center of the ellipsoid.

By way of example, considering arbitrary axes orientation, the ellipsoid-fitting algorithm identifies 9 independent parameters to characterize the ellipsoid based on a general ellipsoid-defining equation:

$$A {x}^{2} + B {y}^{2} + C {z}^{2} + D x y + E x z + F y z + G x + Hy + Iz = 1$$

Three parameters indicate the ellipsoid center (A, B, C), three parameters define the axes (G, H, I), and three parameters define the axes' orientation (D, E, F). Solving this equation following least-squares leads to:

$$AI = {( {J}^{T} J )}^{- 1} {J}^{T} K$$

With Al being the extracted column vector of parameters A to I, J being the input data points translated into a 9-column matrix following the aforementioned generalized ellipsoid formula and K being a vector of 1s. After inverting the resulting matrix, and identifying the eigenvalues and eigenvectors, a description of the generalized ellipsoid is created.

Transformation of the ellipsoid into a sphere allows efficient ranking of input data points based on their distance to the sphere's center. This rank corresponds to the input points' ellipsoid protrusion ranks.

Herein an amino acid residue or residue of a protein that is considered to protrude from (the surface) of a protein or the protein's structure or the cellular surface is termed a “protruding amino acid residue” or “protruding residue”, having a high protrusion rank. A protruding residue may be an amino acid residue that is exposed and/or accessible on the surface of a protein, preferably of a folded protein or natively folded protein, and wherein the protruding residue is preferably accessible to its surrounding, such as to the interaction with or recognition by other molecules or cells, e.g., components of the immune system, antibodies and/or immune cells, preferably B-cells.

In embodiments wherein the protruding residue is part of a protein that is expressed or located (entirely or partially) on the surface of a cell, the protruding residue may be considered to be particularly accessible to other cells and/or components of the immune system, e.g., immune cells, preferably B-cells or antibodies. In the context of embodiments of the invention the terms “protruding amino acid residue” or “protruding residue”, or “amino acid protrusion” or “residue protrusion” may be used interchangeably.

In embodiments a protrusion score, a protrusion rank score or a ‘Snowball score’ may indicate the degree or extend of protrusion of a residue from a protein's and/or cells structure and/or surface. The terms protrusion rank and protrusion score may be used in embodiments herein interchangeably.

The present approach facilitates the “scanning” of a protein structure through the repetitively fitting of numerous ellipsoids to a protein structure's atoms or to subsets of its atoms, thereby determining numerous so called “protrusion ranks” for multiple atoms or for each atom of a protein structure. Thereby the protrusion of an amino acid of a protein or of a sub-structure of a protein (e.g., a part of a protein) can be determined by considering multiple protrusion ranks of numerous atoms or for each atom of a protein structure or sub-structure. In embodiments the atoms' protrusion is ranked based on the ellipsoid's axes with the atom furthest from the center of the ellipsoid being ranked 1.0 and the atom centered in the ellipsoid being ranked 0.0. In embodiments the median of all structure's atoms' protrusion ranks is formed and for each residue the maximum of its atomic protrusion medians is considered the amino acid protrusion rank. Therefore, a high protrusion rank of an amino acid residue indicates a strong protrusion for this residue. Therefore, in embodiments a high protrusion rank of an amino acid residue that is mismatched between a donor and a transplant recipient indicates a higher probability that this residue might be recognized by components of the immune system (constitutes a potential B-cell and/or antibody epitope) of the recipient.

As used herein, “solvent accessible surface residues” relates to any amino acid, for example of an HLA protein, that is determined by a computer-implemented method to be accessible to solvent, for example water, when the protein is in an aqueous environment. The term “surface accessible” may be used interchangeably. Other terms relate to the accessible surface area (ASA) or solvent-accessible surface area (SASA) of a protein, commonly understood as the surface area of a protein that is accessible to a solvent. Various methods for determining the solvent accessible surface residues of a biomolecule are available, for example ASA was first described by Lee & Richards in 1971 and is sometimes called the Lee-Richards molecular surface. ASA is often calculated using a ‘rolling ball’ algorithm, for example as developed by Shrake & Rupley in 1973, which uses a sphere (of solvent) of a particular radius to ‘probe’ the surface of the molecule.

By way of example, the Shrake-Rupley algorithm is a numerical method that draws a mesh of points equidistant from each atom of the molecule and uses the number of these points that are solvent accessible to determine the surface area. The points are drawn at a water molecule's estimated radius beyond the van der Waals radius, which is effectively similar to ‘rolling a ball’ along the surface. All points are checked against the surface of neighboring atoms to determine whether they are buried or accessible. The number of points accessible is multiplied by the portion of surface area each point represents to calculate the ASA. The choice of the ‘probe radius’ does have an effect on the observed surface area, as using a smaller probe radius detects more surface details and therefore reports a larger surface. A typical value is 1.4 Å, which approximates the radius of a water molecule (Shrake, A; Rupley, JA. (1973). “Environment and exposure to solvent of protein atoms. Lysozyme and insulin”. J Mol Biol. 79 (2): 351-71). Alternative methods are described for example in Ali et al (A Review of Methods Available to Estimate Solvent-Accessible Surface Areas of Soluble Proteins in the Folded and Unfolded States, 2014, Curr Protein Pept Sci. 2014; 15(5):456-76).

As used herein, an “HLA protein structure” refers to a three-dimensional (preferably computer representation of the) structure of an HLA protein. HLA protein structures can be obtained via various methods, including analysis of crystal structures.

Protein crystallization is the process of formation of a regular array of individual protein molecules stabilized by crystal contacts. If the crystal is sufficiently ordered, it will diffract. In the process of protein crystallization, proteins are dissolved in an aqueous environment and sample solution until they reach the supersaturated state. Different methods are used to reach that state such as vapor diffusion, microbatch, microdialysis, and free-interface diffusion. Once formed, these crystals can be used in structural biology to study the molecular structure of the protein, particularly for various industrial or medical purposes. Macromolecular structures can be determined from protein crystals using a variety of methods, including X-Ray Diffraction/X-ray crystallography, Cryogenic Electron Microscopy (CryoEM) (including Electron Crystallography and Microcrystal Electron Diffraction (MicroED)), Small-angle X-ray scattering, and Neutron diffraction. Computer representations of the structure can be generated using standard methods established in the art.

As used herein, the term “a structure that has been experimentally determined” relates to an HLA protein structure that has been experimentally assessed and a crystal structure has been determined and analysed.

As used herein, the term “a structure that has not been experimentally determined” relates to an HLA protein structure that has not been experimentally determined or its dataset being published but has been predicted using a computer-implemented method of protein structure prediction. For example, a “protein structure of one or more HLA proteins, for which a structure has not been experimentally determined” may be used interchangeably with “a protein structure of one or more HLA proteins, predicted by a computer-implemented method”.

Protein structure prediction is considered the inference of the three-dimensional structure of a protein from its amino acid sequence. Proteins are chains of amino acids joined together by peptide bonds. Many conformations of this chain are possible due to the rotation of the chain about each alpha-Carbon atom (Ca atom). It is these conformational changes that are responsible for differences in the three-dimensional structure of proteins.

Secondary structure prediction is a set of techniques in bioinformatics that aim to predict the local secondary structures of proteins based on knowledge of amino acid sequence. For proteins, a prediction consists of assigning regions of the amino acid sequence as likely alpha helices, beta strands or turns. The success of a prediction can be determined by comparing it to the results of the DSSP algorithm (or similar e.g., STRIDE) applied to the crystal structure of the protein. Specialized algorithms have been developed for the detection of specific well-defined patterns such as transmembrane helices and coiled coils in proteins. Multiple methods are available to predict structure and a high accuracy can be obtained using e.g., machine learning and sequence alignments. The high accuracy allows the use of the predictions to improve fold recognition and ab initio protein structure prediction, classification of structural motifs, and refinement of sequence alignments.

Tertiary structure prediction provides a predicted structure of the three-dimensional shape of a protein. The tertiary structure will have a single polypeptide chain “backbone” with one or more protein secondary structures, the protein domains. Amino acid side chains may interact and bond in several ways. The interactions and bonds of side chains within a particular protein determine its tertiary structure. Tertiary structure prediction methods typically involve “comparative” or “homology modelling” and “fold recognition” methods. Alternatively, “de novo protein structure prediction” involves an algorithmic process by which protein tertiary structure is predicted from its amino acid primary sequence. Various methods for predicting protein structure are available, such as are reviewed in Pearce et al (JBC, Volume 297, Issue 1, 2021) or Susanty et al (BIO Web of Conferences 41, 04003, 2021).

If a protein of known tertiary structure shares at least 30% of its sequence with a potential homolog of undetermined structure, comparative methods that overlay the putative unknown structure with the known can be utilized to predict the likely structure of the unknown. However, below this threshold three other classes of strategy are used to determine possible structure from an initial model: ab initio protein prediction, fold recognition, and threading. Ab Initio Methods: In ab initio methods, an initial effort to elucidate secondary structures (alpha helix, beta sheet, beta turn, etc.) from primary structure is made by utilization of physicochemical parameters and neural net algorithms. From that point, algorithms predict tertiary folding. One drawback to this strategy is that it is not yet capable of incorporating the locations and orientation of amino acid side chains. Fold Prediction: In fold recognition strategies, a prediction of secondary structure is first made and then compared to either a library of known protein folds, such as CATH or SCOP, or what is known as a “periodic table” of possible secondary structure forms. A confidence score is then assigned to likely matches. Threading: In threading strategies, the fold recognition technique is expanded further. In this process, empirically based energy functions for the interaction of residue pairs are used to place the unknown protein onto a putative backbone as a best fit, accommodating gaps where appropriate. The best interactions are then accentuated in order to discriminate amongst potential decoys and to predict the most likely conformation. The goal of both fold and threading strategies is to ascertain whether a fold in an unknown protein is similar to a domain in a known one deposited in a database, such as the protein databank (PDB). This is in contrast to de novo (ab initio) methods where structure is determined using a physics-based approach en lieu of comparing folds in the protein to structures in a data base.

In one embodiment, the predicted HLA protein structure is provided using an ab initio protein structure prediction. In one embodiment, the predicted HLA protein structure is provided using a fold recognition prediction. In one embodiment, the predicted HLA protein structure is provided using a threading prediction.

Examples of protein structure prediction that may be employed are known in the art, and include techniques described by Jumper et al (Highly accurate protein structure prediction with AlphaFold. Nature 596, 583-589, 2021) or Baek et al (Accurate prediction of protein structures and interactions using a three-track neural network, Science, 19 Aug. 2021, Vol 373, Issue 6557, pp. 871-876).

As used herein, the term “supplementing the predicted HLA protein structures with an HLA-bound peptide” refers to adding a bound peptide to the predicted structure of an HLA protein, such as a structure of an HLA alpha chain and a Beta-2-microglobulin or HLA alpha and beta chain.

As used herein, the term “neural network” refers to a neural network as understood by a skilled person. For example, a neural network is a network or circuit of neurons in an artificial neural network, composed of artificial neurons or nodes, connected by edges. Artificial neural networks (ANNs), also named neural networks (NNs), are based on a collection of connected units or nodes called artificial neurons, which loosely model the neurons in a biological brain.

An artificial neural network preferably consists of a collection of simulated neurons. By way of example, each neuron is a node which is connected to other nodes via links that correspond loosely to biological axon-synapse-dendrite connections. Each link has a weight, which determines the strength of one node's influence on another. The neurons are typically organized into multiple layers, especially in deep learning. Neurons of one layer connect only to neurons of the immediately preceding and immediately following layers. The layer that receives external data is the input layer. The layer that produces the ultimate result is the output layer. In between them may be zero or more hidden layers. Single layer and unlayered networks may also be used. Example neural networks are known to a skilled person and are described in more detail in the Examples below.

As used herein, “training” a neural network refers to the term as commonly understood by a skilled person. By way of example, neural networks learn (or are trained) by processing examples, each of which contains a known “input” and “output,” forming probability-weighted associations between the two, which are stored within the data structure of the net itself. The training of a neural network from a given example is usually conducted by determining the difference between the processed output of the network (often a prediction) and a target output. This difference is the error. The network then adjusts its weighted associations according to a learning rule and using this error value. Successive adjustments will cause the neural network to produce output which is increasingly similar to the target output. After a sufficient number of these adjustments the training can be terminated based upon certain criteria. This may also be referred to as supervised learning. Suitable training modes and software are known to a skilled person and described in the Examples below.

According to the present invention, the term “predict” means any statement about a possible outcome or state based on any given analysis. By way of example, a “prediction” in the sense of the present invention may represent an assessment of “predicted” surface protruding residues. The prediction is therefore a computer-implemented assessment of the likelihood of surface protrusion of any given amino acid in the HLA amino acid sequence. The computer-implemented method or software is capable of determining, according to any given set of parameters, the surface (solvent) accessibility in the computer representation, whereby a prediction is made as to whether the amino acid is indeed accessible in a biological system.

The term “Immune response” in the context of the present invention relates to an immune response as commonly understood by one skilled in the art. An immune response may be understood as a response from a part of the immune system to an antigen that occurs when the antigen is identified as foreign, which preferably subsequently induces the production of antibodies and/or lymphocytes capable of destroying or immobilising the “foreign” antigen or making it harmless. The immune response of the present invention may relate either to a response of the immune system of the recipient against the transplanted material, or an immune response effected by cells of the transplanted cells, tissues, or organs, whereby for example in GVHD T cells of the transplanted material react against and/or attack recipient antigens or tissue. The immune response may be a defense function of the recipient that protects the body against foreign matter, such as foreign tissue, or a reaction of immune cells of the transplanted material against recipient cells or tissue.

The human leukocyte antigen (HLA) system is the major histocompatibility complex (MHC) in humans. The super locus contains a large number of genes related to immune system function in humans. This group of genes resides on chromosome 6, and encodes cell-surface antigen-presenting proteins and has many other functions. The proteins encoded by certain genes are also known as antigens, as a result of their historic discovery as factors in organ transplants. The major HLA antigens are essential elements for immune function. HLAs corresponding to MHC class I (A, B, and C) present peptides from inside the cell (including viral peptides if present). These peptides are produced from digested proteins that are broken down in the proteasomes. In general, these particular peptides are small polymers, about 9 amino acids in length. Foreign antigens attract killer T-cells (also called CD8 positive- or cytotoxic T-cells) that destroy cells. HLAs corresponding to MHC class II (DP, DM, DOA, DOB, DQ, and DR) present antigens from outside of the cell to T-lymphocytes. These particular antigens stimulate the multiplication of T-helper cells, which in turn stimulate antibody-producing B-cells to produce antibodies to that specific antigen.

MHC loci are some of the most genetically variable coding loci in mammals, and the human HLA loci are no exception. Most HLA loci show a dozen or more allele-groups for each locus. Six loci have over 2000 alleles that have been detected in the human population. Of these, the most variable are HLA-B and HLA-DRB1. Given the heterodimeric structure of Class II alleles, combinations of alpha and beta chains alter peptide binding specificity, resulting in super-linear peptide binding signatures.

An “allele” is a variant of the nucleotide (DNA) sequence at a locus, such that each allele differs from all other alleles by at least one (single nucleotide polymorphism, SNP) position. Most of these changes result in a change in the amino acid sequences that result in slight to major functional differences in the protein.

“HLA” refers to the human leukocyte antigen locus on chromosome 6p21, consisting of HLA genes (HLA-A, HLA-B, HLA-C, HLA-DRB1, HLA-DQB1, etc. . . . ) that are used to determine the degree of matching, for example, between a recipient and a donor of a tissue graft. “HLA allele” means a nucleotide sequence within a locus on one of the two parental chromosomes.

“HLA typing” means the identification of an HLA allele of a given locus (HLA-A, HLA-B, HLA-C, HLA-DRB1, HLA-DQB1, etc., . . . ). Samples may be obtained from blood or other body samples from donor and/or recipient, which may subsequently be analysed.

Serotyping (identification of the HLA protein on the surface of cells using antibodies) is the original method of HLA typing and is still used by some centres. Phenotyping relates to the serological approach for typing. This method is limited in its ability to define HLA polymorphism and is associated with a risk of misidentifying the HLA type.

Modern molecular methods are more commonly used to genotype an individual. With this strategy, PCR primers specific to a variant region of DNA are used (called PCR SSP). If a product of the right size is found, the assumption is that the HLA allele/allele group has been identified. PCR-SSO may also be used incorporating probe hybridisation. Reviews of technical approaches towards HLA typing are provided in Erlich H, Tissue Antigens, 2012 July; 80(1):1-11 and Dunn P, Int J Immunogenet, 2011 December; 38(6):463-73.

Gene sequencing may be applied, and relates to traditional methods, such as Maxam-Gilbert sequencing, Chain-termination methods, advanced methods and de novo sequencing such as shotgun sequencing or bridge PCR, or the so-called “next-generation” methods, such as massively parallel signature sequencing (MPSS), 454 pyrosequencing, Illumina (Solexa) sequencing, SOLiD sequencing, nanopore sequencing or other similar methods.

With respect to HLA typing, samples obtained from the donor themselves and/or from donor material before, during or after isolation/preparation for transplantation, may be used for HLA-typing and subsequent comparison of HLA-typing data between subjects. For example, HLA-typing of the donor themselves, for example by analyzing a saliva, blood or other bodily fluid sample for HLA information, may occur, and optionally additionally or alternatively, the material obtained from the donor intended for transplantation (donor material) may be analyzed for the same and/or complementary HLA type characteristics during HLA-typing.

An “HLA-mismatch” is defined as the identification of different alleles in donor and recipient, which are present at any given loci. Preferably, the HLA mismatch is a difference in protein sequence between two subjects.

According to the present invention, the term “two-field specific HLA protein typing” relates to the standardised HLA naming system developed in 2010 by the WHO Committee for Factors of the HLA System. According to the HLA naming system, the nomenclature is as follows: HLA-Gene*Field1:Field2:Field3:Field4-Suffix. Gene relates to the HLA locus (A, B, C, DRB1, DQB1, etc. . . . ). Field 1 relates to the allele group. Field 2 relates to the specific HLA protein. Field 3 relates to a synonymous DNA substitution within the coding region. Field 4 is used to show differences in non-coding regions. The suffix is used to denote particular characteristics in gene expression.

The “two-field” within “two-field specific HLA protein typing” relates to the presence of HLA typing information for Fields 1 and 2 according to the standardized WHO nomenclature used herein. This typing is commonly referred to as “high resolution typing” or “specific HLA protein typing”, as the typing provides information on the specific protein sequence relevant for the immune responses dealt with herein.

The term “HLA allele group” typing relates to typing information that is provided for only the HLA gene (locus) and Field1. This typing is commonly referred to as “low resolution” typing. According to the present invention any serotype would be converted to a molecular “low resolution” typing.

The term “intermediate resolution” or “medium resolution” typing relates to some kinds of typing information where the presence of some alleles, or allele strings, have been defined in the patient/donor by the typing method used. The NMDP nomenclature is one method of describing this level of typing. For example A*01:AB relates to alleles 01:01 or 01:02 (01:01/01:02) and the patient/donor is one of these two alleles. The NMDP allele codes are in some cases generic, wherein one code may encompass a number of possible HLA sequences (alleles) at any given HLA locus. Although the donor material may be HLA typed, a number of NMDP codes do not provide information on which specific protein is in fact present at any given HLA locus. This form of typing information is referred to as intermediate resolution typing.

The “donor” is commonly understood to be an individual or multiple individuals (for example in the case of where multiple samples or preparations, such as cord blood units (CBUs) are required for an effective therapeutically relevant amount of donor material for the transplantation) who provide donor material, or in other words biological material, for transplantation in the recipient. References to the donor, or HLA-typing of the donor, may also refer to donor material, or HLA-typing of the donor material, respectively.

The subjects of the invention may be either the donor or recipient, for example where two subjects are compared for HLA mismatches, a donor and recipient are preferably the two subjects.

The subject “recipient” of the method is typically a mammal, preferably a human. The recipient is typically a patient suffering from a disorder characterised by the need for transplantation, such as organ failure necessitating a transplant.

By the term “organ”, it is meant to include any bodily structure or group of cells containing different tissues that are organized to carry out a specific function of the body, for example, the heart, lungs, brain, kidney skin, liver, bone marrow, etc. In one embodiment the graft is an allograft, i.e. the individual and the donor are of the same species. The subject may also suffer from a condition that could be treated by the transplantation of cells, even when the disorder itself is not defined by a lack or loss of function of a particular subset of endogenous cells. Some disorders may be treatable by the transplantation of certain kinds of stem cells, whereby the native or endogenous pool of such cells are not necessarily non-functional in the recipient.

The method of the invention is particularly applicable to patients who are about to receive or are predicted to require a transplant, to predict the likelihood of unwanted immune response, such as origin graft damage or rejection, and/or immune origin damage to non-graft tissue. For example, the patient may be expected to receive a transplant in the next one, two, three, four, five, six, or twelve months. Alternatively, the assay is particularly applicable to individuals who have received a transplant to predict the likelihood of immune origin graft damage or rejection, and/or immune origin damage to non-graft tissue. Post-transplant, the method is particularly applicable to patients who show evidence of chronic organ dysfunction (of the graft organ) or possible graft versus host disease (GVHD), particularly chronic GVHD and particularly in cases wherein the graft is a bone marrow transplant.

Transplantation is the moving of biological material, such as cells, tissue or an organ, from one body (donor) to another (recipient or patient), or from a donor site to another location on the patient's own body, or from stored donor material to a recipient's body. Preferably the transplantation is carried out for the purpose of a beneficial medical effect in the recipient, for example by replacing the recipient's damaged or absent organ, or for the purpose of providing stem cells, other cells, tissues or organs capable of providing a therapeutic effect.

Allogeneic transplantation or Allotransplantation is the transplantation of biological material, such as cells, tissues, or organs, to a recipient from a genetically non-identical donor of the same species. The transplant is called an allograft, allogeneic transplant, or homograft. Most human tissue and organ transplants are allografts. Allografts can either be from a living or cadaveric source. Generally, organs that can be transplanted are the heart, kidneys, liver, lungs, pancreas, intestine, and thymus. Tissues include bones, tendons (both referred to as musculoskeletal grafts), cornea, skin, heart valves, nerves and veins.

The invention also encompasses use of the method in the context of screening biological material, such as organs, cells, or tissues produced via regenerative medicine, for example reconstructed donor material that has been constructed ex vivo and is intended for transplantation. Stem cell technologies enable the production of a number of medically relevant cell types or tissues ex vivo. The present invention could therefore also be applied in screening allogeneic material that has been produced by biotechnological and/or tissue engineering methods for its suitability in transplantation.

The invention encompasses the assessment of risk of an immune reaction, preferably a pre-transplantation risk assessment. In embodiments, for stem cells, any given stem cell may be considered as donor material intended for transplantation. For example, hematopoietic stem cell transplantation (HSCT) is the transplantation of multipotent hematopoietic stem cells, usually derived from bone marrow, peripheral blood, or umbilical cord blood. It is a medical procedure common in the fields of hematology and oncology, most often performed for patients with certain cancers of the blood or bone marrow, such as multiple myeloma or leukemia. In these cases, the recipient's immune system is usually destroyed with radiation or chemotherapy before the transplantation. Infection and graft-versus-host disease are major complications of allogenic (also referred to as allogeneic) HSCT.

Stem cells are to be understood as undifferentiated biological cells, that can differentiate into specialized cells and can divide (through mitosis) to produce more stem cells. Highly plastic adult stem cells are routinely used in medical therapies, for example in bone marrow transplantation. Stem cells can now be artificially grown and transformed (differentiated) into specialized cell types with characteristics consistent with cells of various tissues such as muscles or nerves through cell culture. The potential stem cell transplantation may relate to any given stem cell therapy, whereby a number of stem cell therapies exist. Medical researchers anticipate that adult and embryonic stem cells will soon be able to treat cancer, Type 1 diabetes mellitus, Parkinson's disease, Huntington's disease, Celiac disease, cardiac failure, muscle damage and neurological disorders, and many others.

Also known as somatic stem cells and germline stem cells, stem cells can be found in children, as well as adults. Pluripotent adult stem cells are rare and generally small in number but can be found in a number of tissues including umbilical cord blood. Bone marrow has been found to be one of the rich sources of adult stem cells which have been used in treating several conditions including Spinal cord injury, Liver Cirrhosis, Chronic Limb Ischemia and End-stage heart failure. Adult stem cells may be lineage-restricted (multipotent) and are generally referred to by their tissue origin (mesenchymal stem cell, adipose-derived stem cell, endothelial stem cell, dental pulp stem cell, etc.).

Multipotent stem cells are also found in amniotic fluid. These stem cells are very active, expand extensively without feeders and are not tumorigenic. Amniotic stem cells are multipotent and can differentiate in cells of adipogenic, osteogenic, myogenic, endothelial, hepatic and also neuronal lines. It is possible to collect amniotic stem cells for donors or for autologuous use.

Cord blood-derived multipotent stem cells display embryonic and hematopoietic characteristics. Phenotypic characterization demonstrates that (CB-SCs) display embryonic cell markers (e.g., transcription factors OCT-4 and Nanog, stage-specific embryonic antigen (SSEA)-3, and SSEA-4) and leukocyte common antigen CD45, but that they can be negative for blood cell lineage markers. Additionally, CB-SCs display very low immunogenicity as indicated by expression of a very low level of major histocompatibility complex (MHC) antigens and failure to stimulate the proliferation of allogeneic lymphocytes.

HSC are typically available from bone marrow, Peripheral blood stem cells, Amniotic fluid, or Umbilical cord blood. In the case of a bone marrow transplant, the HSC are removed from a large bone of the donor, typically the pelvis, through a large needle that reaches the center of the bone. The technique is referred to as a bone marrow harvest and is performed under general anesthesia. Peripheral blood stem cells are a common source of stem cells for allogeneic HSCT. They can be collected from the blood through a process known as apheresis. The donor's blood is withdrawn through a sterile needle in one arm and passed through a machine that removes white blood cells. The red blood cells may be returned to the donor. The peripheral stem cell yield may be boosted with daily subcutaneous injections of Granulocyte-colony stimulating factor, serving to mobilize stem cells from the donor's bone marrow into the peripheral circulation.

It is also possible to extract hematopoietic stem cells from amniotic fluid. Umbilical cord blood is obtained from an infant's Umbilical Cord and Placenta after birth. Cord blood has a higher concentration of HSC than is normally found in adult blood. However, the small quantity of blood obtained from an Umbilical Cord (typically about 50 ml.) makes it more suitable for transplantation into small children than into adults. Multiple units could however be used. Newer techniques using ex-vivo expansion of cord blood units or the use of two cord blood units from different donors allow cord blood transplants to be used in adults. Cord blood can be harvested from the umbilical cord of a child being born.

Unlike other organs, bone marrow cells can be frozen (cryopreserved) for prolonged periods without damaging too many cells. This is a necessity with autologous HSC because the cells are generally harvested from the recipient months in advance of the transplant treatment. In the case of allogeneic transplants, fresh HSC are preferred in order to avoid cell loss that might occur during the freezing and thawing process. Allogeneic cord blood is typically stored frozen at a cord blood bank because it is only obtainable at the time of childbirth. To cryopre-serve HSC, a preservative, DMSO, must be added, and the cells must be cooled very slowly in a controlled-rate freezer to prevent osmotic cellular injury during ice crystal formation. HSC may be stored for years in a cryofreezer, which typically uses liquid nitrogen. In light of this, the invention may relate to typing and risk assessment of donor material already stored as described herein, before being considered for transplantation.

The invention encompasses the assessment of risk of an immune reaction, preferably a pre-transplantation risk assessment, whereby any given organ or tissue may be considered as donor material intended for transplantation. For example, kidney transplantation or renal transplantation is the organ transplant of a kidney into a patient, for example with end-stage renal disease. Kidney transplantation is typically classified as deceased-donor (formerly known as cadaveric) or living-donor transplantation depending on the source of the donor organ. Living-donor renal transplants are further characterized as genetically related (living-related) or non-related (living-unrelated) transplants, depending on whether a biological relationship exists between the donor and recipient.

Alloreactivity is defined as the reaction of a lymphocyte or antibody with an alloantigen, which may be understood as an antigen from foreign material. Alloantigen recognition may occur via direct or indirect alloantigen recognition, by which T cells may recognize alloantigens and potentially lead to transplant rejection after an organ transplantation. Furthermore, alloantigen recognition may occur via T cell dependent or T cell independent recognition, by which B cells may recognize alloantigens and potentially lead to transplant rejection after an organ transplantation. In T cell dependent B cell activation, an immune response is triggered and reinforced by a conformational loop between B cells' alloantigen presentation and T cell activation. This concept is described as “linked recognition”.

Graft-versus-host disease (GVHD) is a relatively common complication following an allogeneic cell, tissue or organ transplant. It is commonly associated with stem cell or bone marrow transplant but the term also applies to other forms of tissue graft or organ transplant. Immune cells (typically white blood cells) in the tissue (the graft) recognize the recipient (the host) as “foreign”. The transplanted immune cells then attack the host's body cells. GVHD can also occur after a blood transfusion if the blood products used have not been irradiated.

The method, system or computer product of the invention may in some embodiments comprise one or more conventional computing devices having a processor, an input device such as a keyboard or mouse, memory such as a hard drive and volatile or nonvolatile memory, and computer code (software) for the functioning of the invention. The computers may also comprise a programmable printed circuit board, microcontroller, or other device for receiving and processing data signals such as those received from the local controllers, programmable manufacturing equipment, programmable material handling equipment, and robotic manipulators.

The method, system or computer product may comprise one or more conventional computing devices that are pre-loaded with the required computer code or software, or it may comprise custom-designed hardware. The system may comprise multiple computing devices which perform the steps of the invention. In certain embodiments, a plurality of clients such as desktop, laptop, or tablet computers can be connected to a server such that, for example, multiple users can enter their orders for personalized products at the same time. The computer system may also be networked with other computers over a local area network (LAN) connection or via an Internet connection. The system may also comprise a backup system which retains a copy of the data obtained by the invention. The data connections of step e) may be conducted or configured via any suitable means for data transmission, such as over a local area network (LAN) connection or via an Internet connection, either wired or wireless.

A client or user computer can have its own processor, input means such as a keyboard, mouse, or touchscreen, and memory, or it may be a dumb terminal which does not have its own independent processing capabilities, but relies on the computational resources of another computer, such as a server, to which it is connected or networked. Depending on the particular implementation of the invention, a client system can contain the necessary computer code to assume control of the system if such a need arises. In one embodiment, the client system is a tablet or laptop.

The components of the computer system may be conventional, although the system may be custom-configured for each particular implementation. The computer system may run on any particular architecture, for example, personal/microcomputer, minicomputer, or mainframe systems. Exemplary operating systems include Apple Mac OS X and iOS, Microsoft Windows, and UNIX/Linux; SPARC, POWER and Itanium-based systems; and z/Architecture. The computer code to perform the invention may be written in any programming language or model-based development environment, such as but not limited to C/C++, C#, Objective-C, Java, Basic/VisualBasic, MATLAB, Simulink, StateFlow, Lab View, or assembler. The computer code may comprise subroutines which are written in a proprietary computer language which is specific to the manufacturer of a circuit board, controller, or other computer hardware component used in conjunction with the invention.

The method, system or computer product, and database of the invention, can employ any kind of file format which is used in the industry. For example, a digital representation of a protein sequence, protein structure, or other computer-implemented aspect of the invention, can be stored in a proprietary format, DXF format, XML format, or other format for use by the invention. Any suitable computer readable medium may be utilized. The computer-usable or computer-readable medium may be, for example but not limited to, an electronic, magnetic, optical, electromagnetic, infrared, or semiconductor system, apparatus, device, or propagation medium. More specific examples (a non-exhaustive list) of the computer-readable medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a transmission media such as those supporting the Internet or an intranet, cloud storage or a magnetic storage device.

The invention therefore further relates, in related aspects, to a computer product, such as a software, or kit comprising computer software, or to an online portal offering a software-based service, for producing a database of predicted surface protruding residues for multiple human leukocyte antigen (HLA) proteins, comprising:
a. Providing one or more HLA protein amino acid sequences, for which a protein structure has been experimentally determined,
b. Predicting HLA protein structure of one or more HLA proteins, for which a structure has not been experimentally determined,
c. Supplementing the predicted HLA protein structures of b. with an HLA-bound peptide, if the structure of b. does not exhibit an HLA-bound peptide,
d. Determining surface-protruding residues of each HLA protein structure of a. and c. using the method according to the invention for determining an amino acid residue protruding from the surface of a human leukocyte antigen (HLA) protein structure,
e. Generating a database of surface protruding residues for the HLA proteins of a) and c) and HLA proteins additional to those of a) to c), comprising employing a neural network trained on amino acids sequences of the HLA proteins of a. and c. and the corresponding surface-protruding residues determined in d).

Embodiments of the present invention are described below with reference to flowchart figures and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions, such as by modules. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

### FIGURES

The invention is further described by the following figures. These are not meant to restrict the scope of the invention but represent preferred embodiments of aspects of the invention provided for greater illustration of the invention described herein.

### BRIEF DESCRIPTION OF THE FIGURES

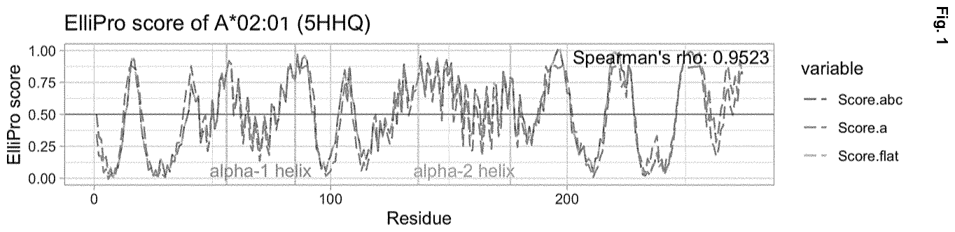

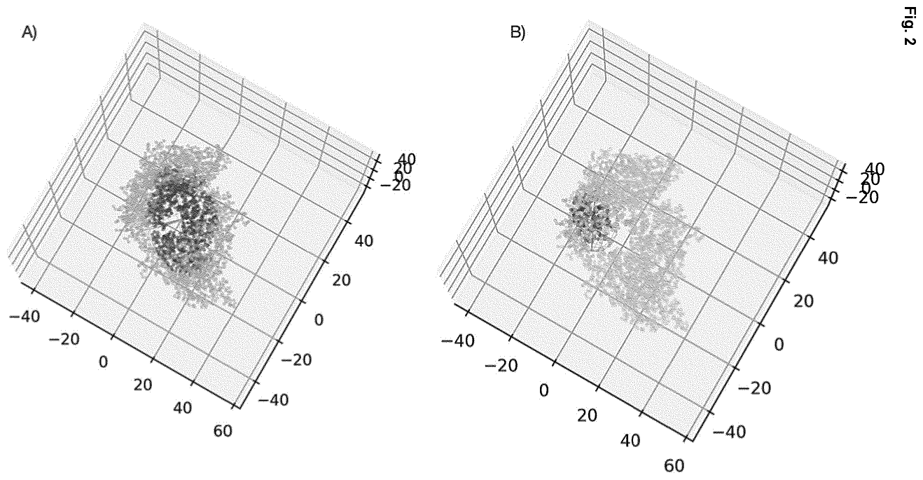

FIG. 1: ElliPro scores of A*02:01 (PDBID 5HHQ).

FIG. 2: A) HLA is not well described by single Ellipsoids.

FIG. 3: Example of fitting an ellipsoid to non-hydrogen atoms of HLA-A*02:01 (PDB 3UTQ).

FIG. 4: Data flow chart of experimental and predicted HLA structures considered in surface area and protrusion prediction.

FIG. 5: Repeated local protrusion strongly correlates with the ElliPro score. Snowball's mean protrusion rank is however higher than average ElliPro scores.

FIG. 6: Sphere radii impact sensitivity of repeated ellipsoid fitting on local structural changes.

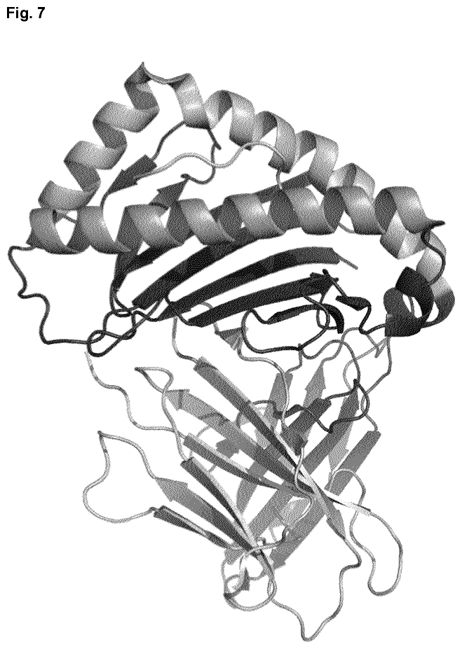

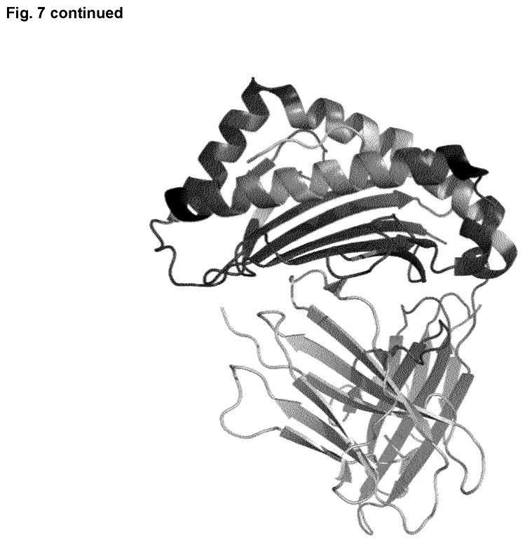

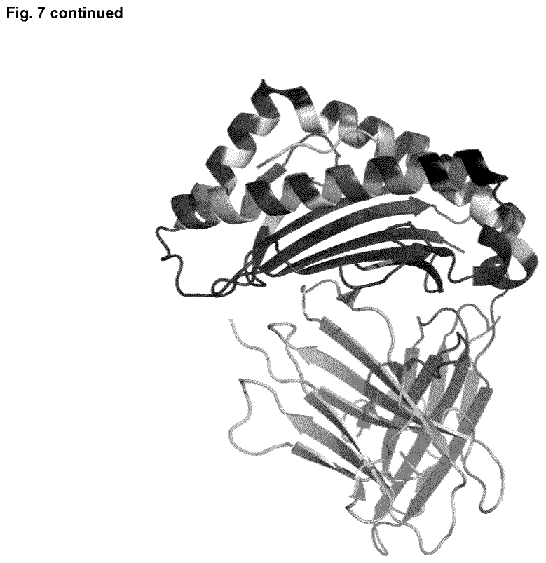

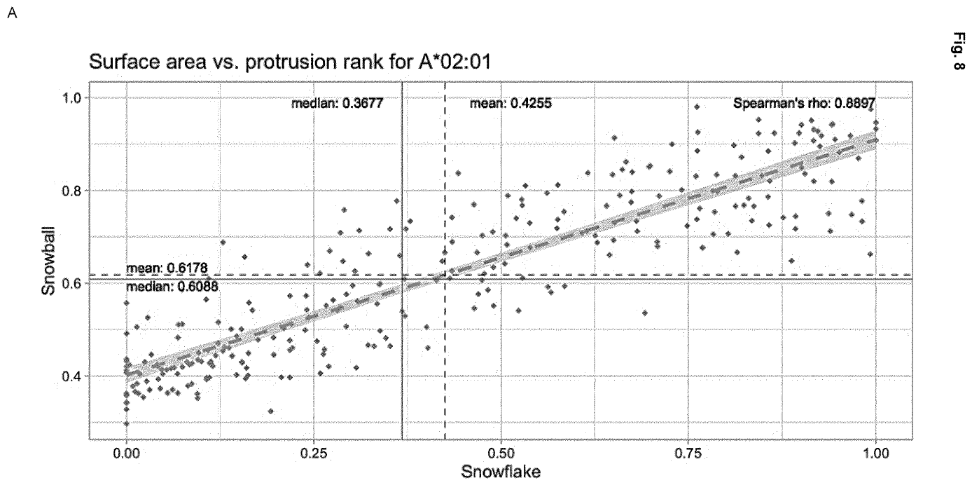

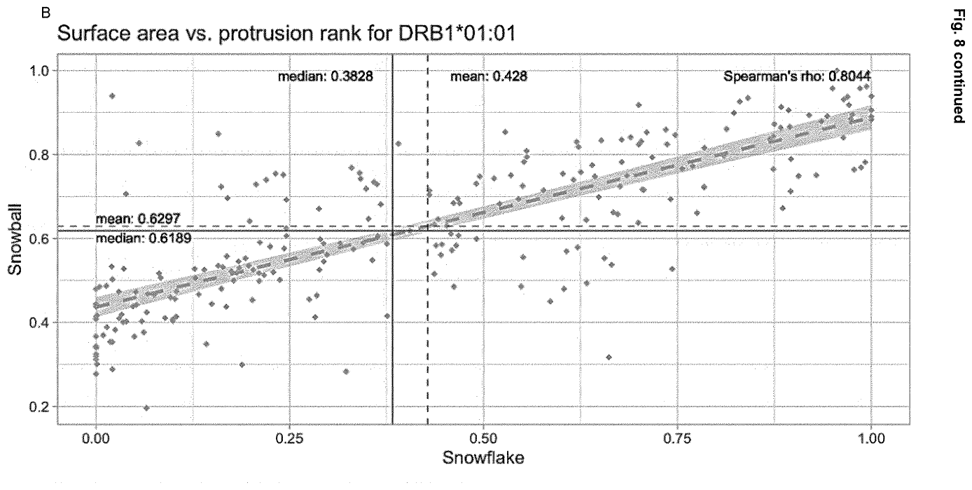

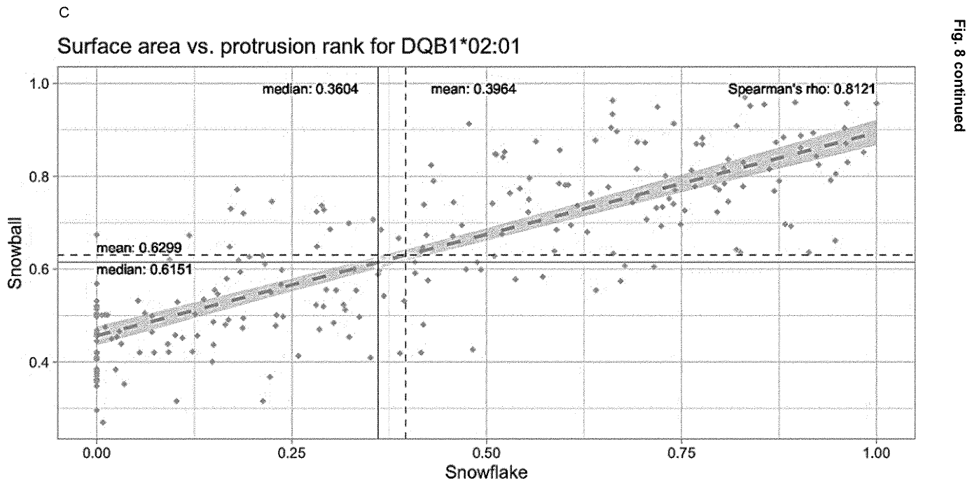

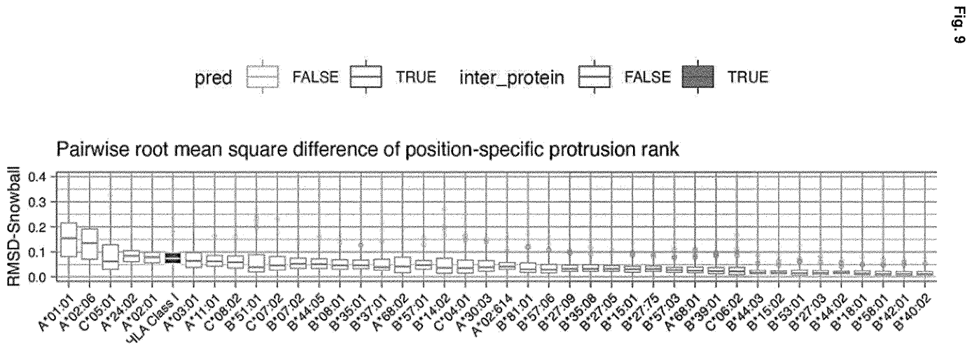

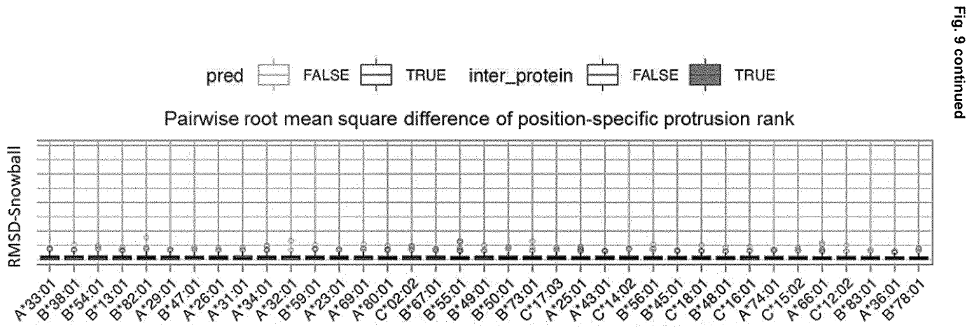

FIG. 7: Crystal Structure of HLA-A*0201.

FIG. 8: Scatterplot of Snowball (protrusion rank) and Snowflake (solvent accessibility) scores for A*02:01, DRB1*01:01 and DQB1*02:01.

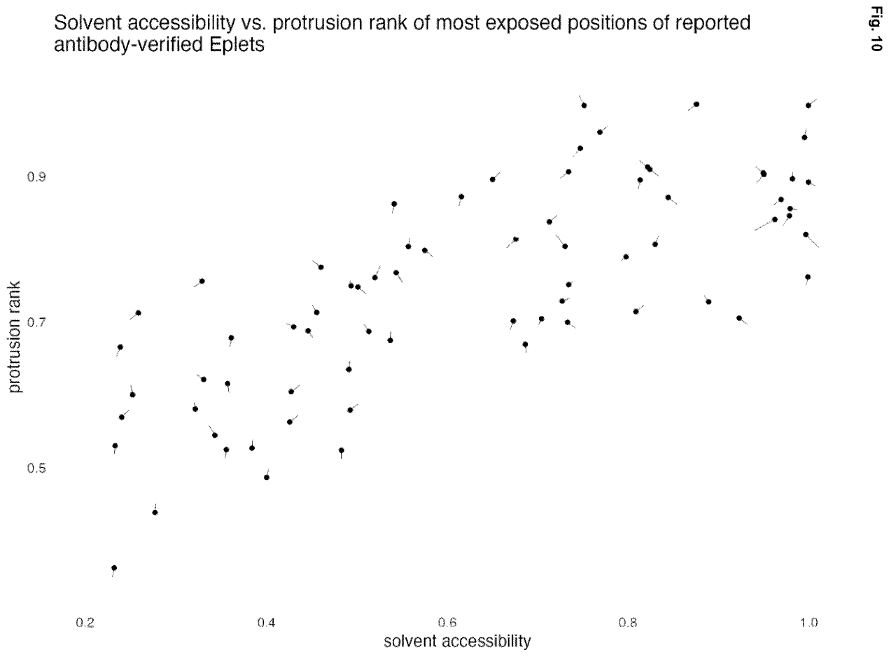

FIG. 9: Pairwise RMSDSNB of experimental structures.

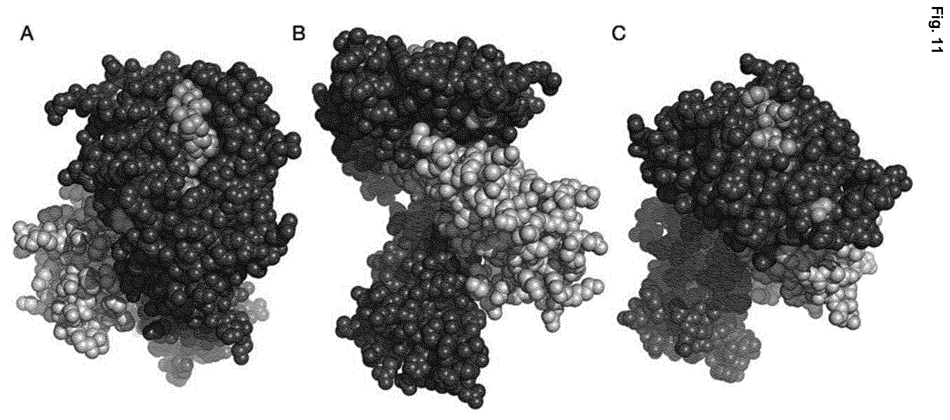

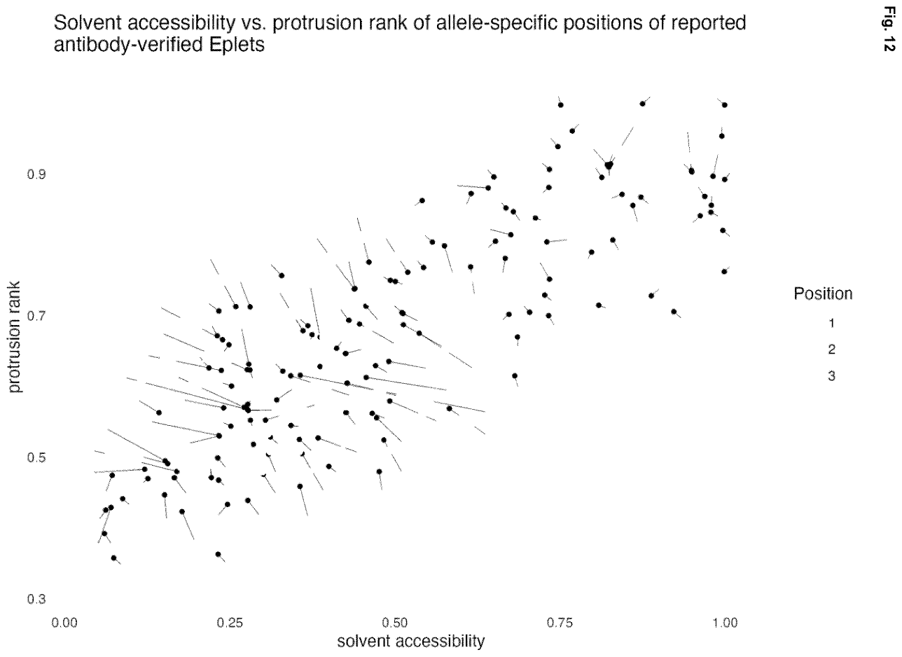

FIG. 10: Mean Snowflake (x axis) and Snowball (y axis) scores of most exposed positions of each reported antibody-verified Eplet based on HLA proteins carrying the respective Eplet.

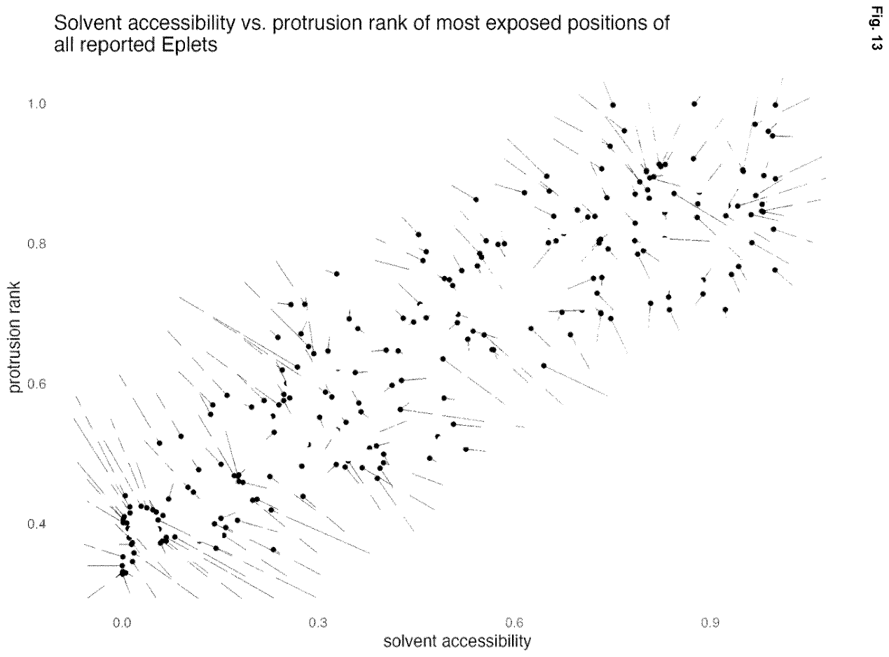

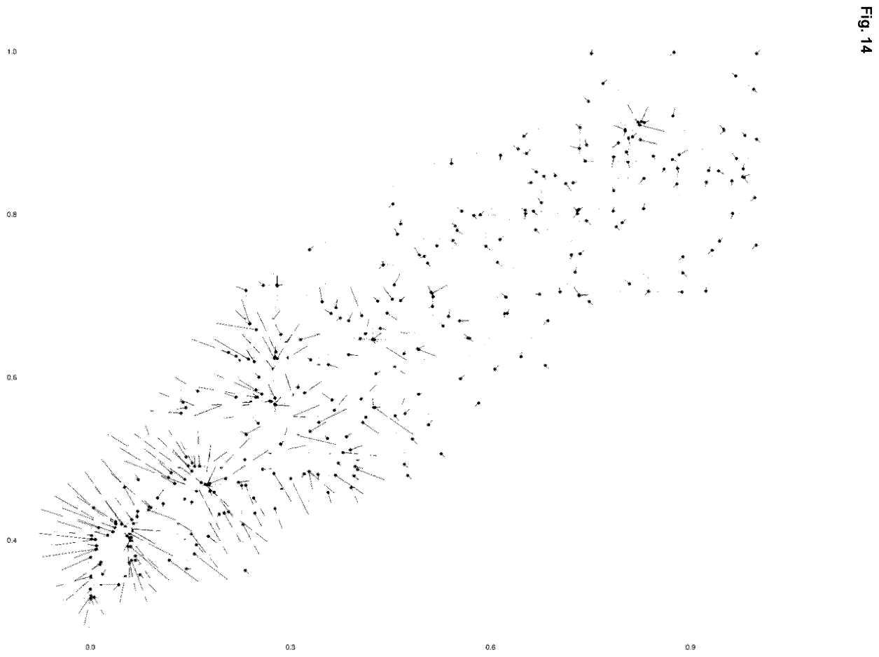

FIG. 11: Rendered A*02:01 structure (PDB 3UTQ) with alpha chain colored in teal, beta-2-microglobulin colored in gray, bound peptide colored in light grey.

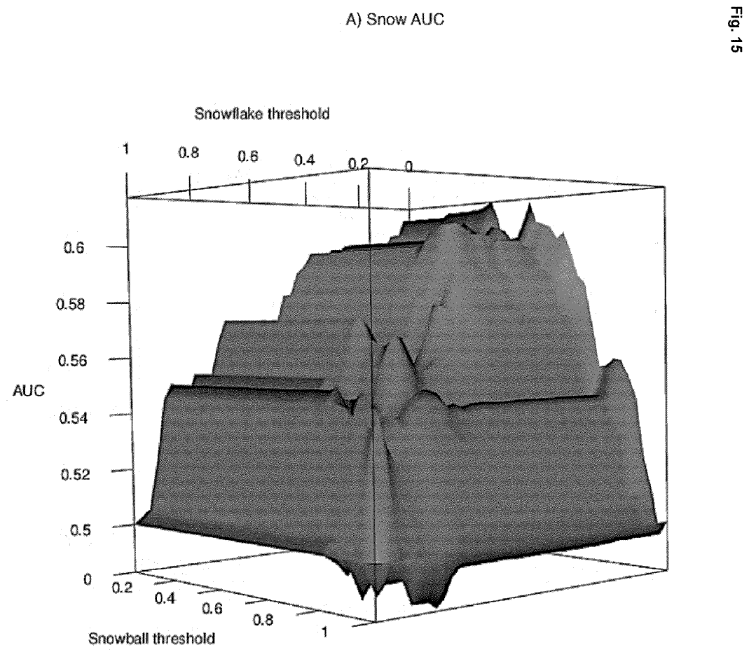

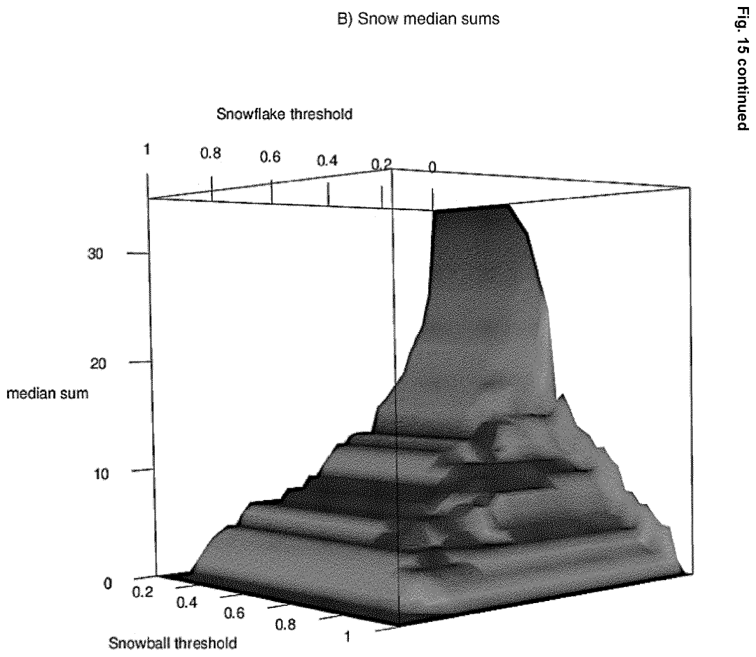

FIG. 12: Mean Snowflake (x axis) and Snowball (y axis) scores of antibody-verified Eplets based on HLA proteins carrying the respective Eplet.

FIG. 13: Mean Snowflake (x axis) and Snowball (y axis) scores of most exposed positions of each reported non-antibody-verified Eplet based on HLA proteins carrying the respective Eplet.

FIG. 14: Mean Snowflake (x axis) and Snowball (y axis) scores of all reported Eplets based on HLA proteins carrying the respective Eplet.

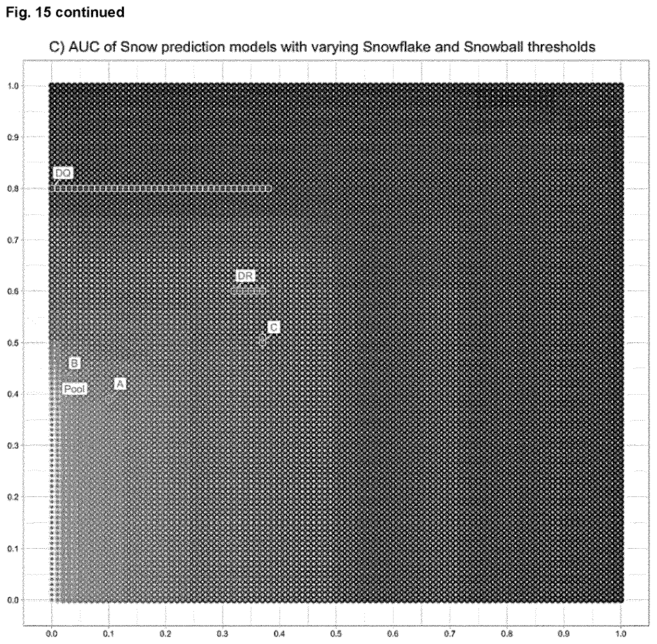

FIG. 15: AUCs for pooled CSA data of the PC considering various thresholds for Snowflake (x axis) and Snowball (y axis).

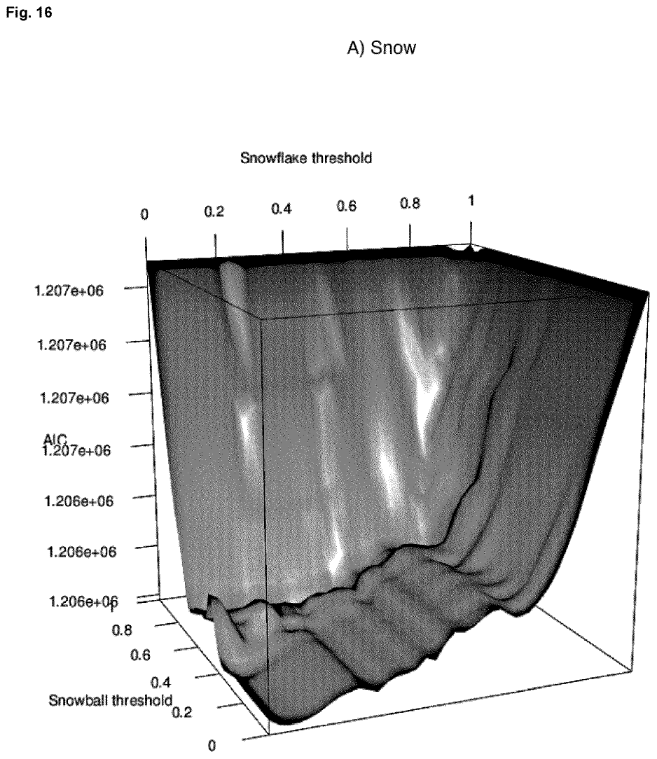

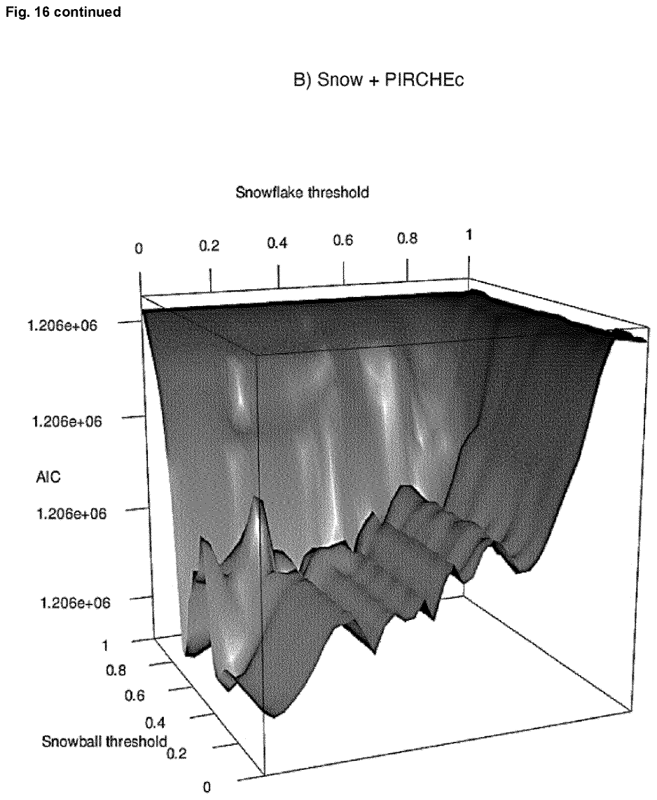

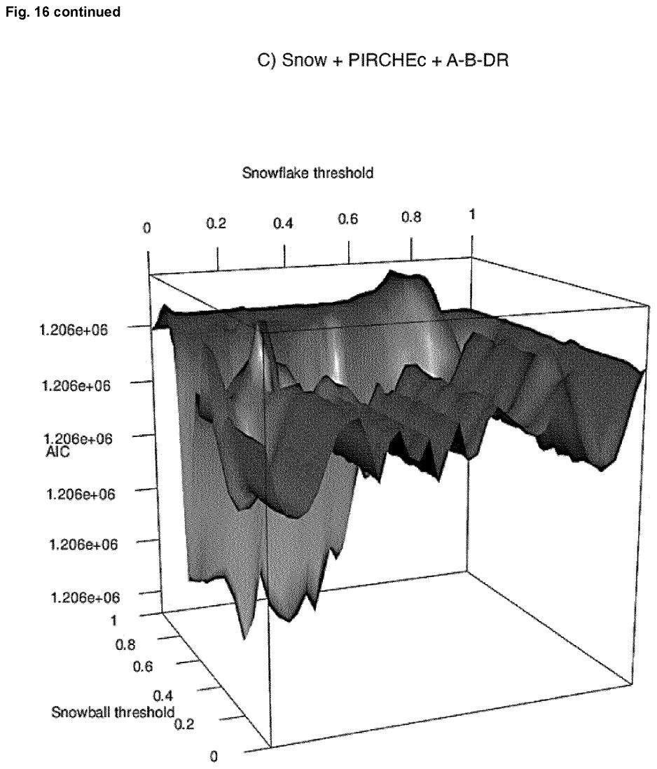

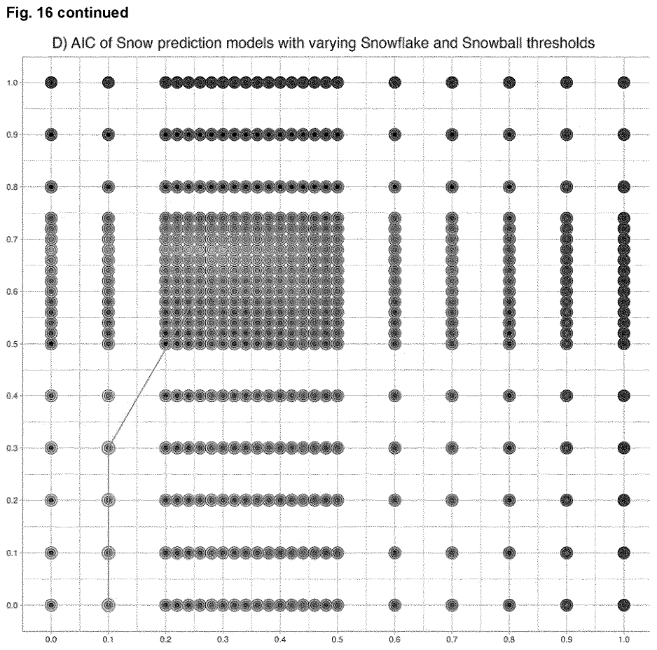

FIG. 16: Akaike Information Criterion values (z axis) for Cox proportional hazard model considering models of (A) log(Snow+1), (B) log(Snow+1) and log(PIRCHEc+1), and (C) log(Snow+1) and log (PIRCHEc+1) and A-B-DR scores at varying Snowflake (x-axis) and Snowball (y-axis) thresholds.

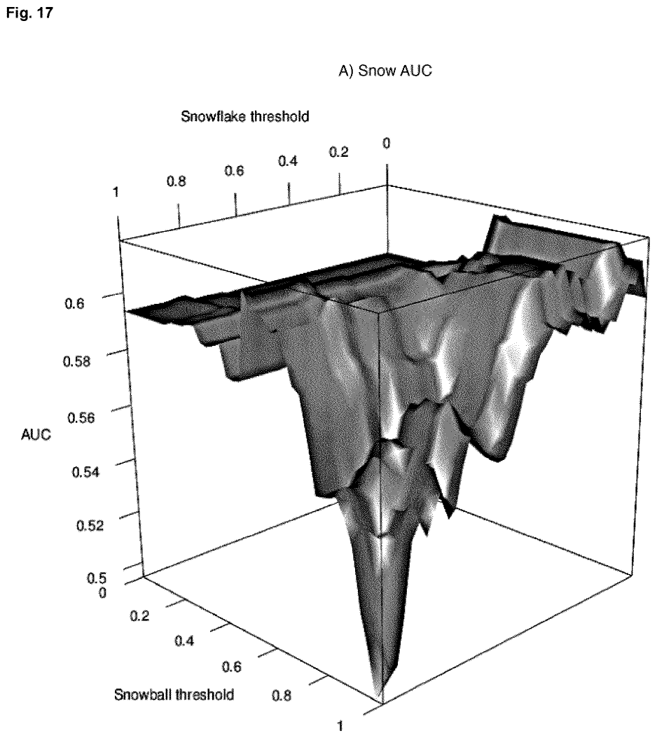

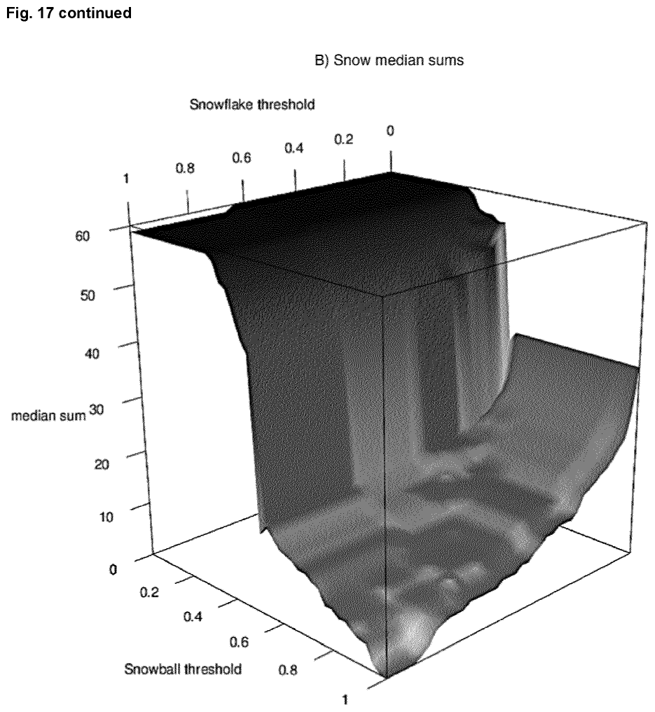

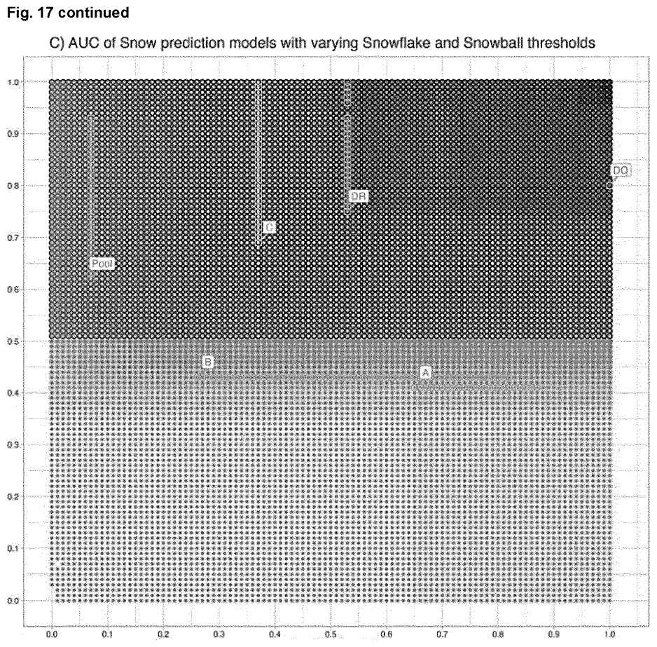

FIG. 17: AUCs for pooled CSA data of the PC considering various thresholds for Snowflake (x axis) and Snowball (y axis) with Snow being configured as either exceeding Snowflake thresholds or Snowball thresholds or both.

### DETAILED DESCRIPTION OF THE FIGURES

FIG. 1: ElliPro scores of A*02:01 (PDBID 5HHQ). Transmitting chains A, B and C (dark gray), only A (grey) and A, B and C aggregated into a virtual single chain yields highly correlated protrusion rank scores. The regions encoding the alpha helices show small fluctuations in protrusion rank, yet follow a superordinate curve.

FIG. 2: A) HLA is not well described by single Ellipsoids (dark grey, center of structure). Consequently, very large protruding regions are defined (outer circle of structure), compared to buried regions. B) Locally fitted ellipsoids (dark grey, left part of structure) better adapt for HLA structures. Plots show all non-hydrogen atoms of A*02:01 (PDBID 5HHQ) as points with HLA alpha (dark grey, left part of structure), beta-2 (light grey, right part of structure) and peptide chains (grey, left part of structure).

FIG. 3: Example of fitting an ellipsoid to non-hydrogen atoms of HLA-A*02:01 (PDB 3UTQ). Center atom 560, amino acid position 72, radius of 15 Å. Alpha-chain atoms at top of the structure, beta-1-microglobulin at the center of structure, peptide in purple. A) Center atom 560 highlighted in dark grey (top left of structure). B) Selected atoms in a 15 Å radius highlighted in dark grey (top left of structure). C) Fitted ellipsoid shown in dark grey (top left of structure), selected atoms inside the ellipsoid in teal, selected atoms outside the ellipsoid in grey (top left of structure). D) Coordinates of selected atoms transformed to spherical coordinates to rank distance to the center.

FIG. 4: Data flow chart of experimental and predicted HLA structures considered in surface area and protrusion prediction.

FIG. 5: Repeated local protrusion strongly correlates with the ElliPro score. Snowball's (method according to the invention) mean protrusion rank is however higher (in graph on top of) than average ElliPro scores.

FIG. 6: Sphere radii impact sensitivity of repeated ellipsoid fitting on local structural changes. Radii are 3, 9, 15, 25, 35, 50, 100 Å (angstrom). Small radii (e.g., 3 Å) are susceptible to overfitting, whereas large radii consider solvent-inaccessible residues as protruding compared to the overall structure (e.g. 50 Å or 100 Å), similar to ElliPro's global protrusion ranks.

FIG. 7: The present method is highly sensitive to distinguish inward facing amino acids and outbound amino acids (see also FIG. 6) of the characteristic helix structures of the HLA binding groove. The present Figure shows cartoons of A*02:01 (PDBID 5HHQ; Crystal Structure of HLA-A*0201).

FIG. 8: Scatterplot of Snowball (protrusion rank) and Snowflake (solvent accessibility) scores for all amino acid positions of (A) A*02:01, (B) DRB1*01:01 and (C) DQB1*02:01. Results of Example 1. Purple dashed line indicates linear model including confidence intervals, black dashed lines indicate means, black solid lines indicate medians.

FIG. 9: Pairwise RMSDSNB of experimental structures.

FIG. 10: Mean Snowflake (x axis) and Snowball (y axis) scores of most exposed positions of each reported antibody-verified Eplet based on HLA proteins carrying the respective Eplet. Error bars represent the respective score's standard deviations. The Figure shows the data without labels.

FIG. 11: Rendered A*02:01 structure (PDB 3UTQ) with alpha chain colored in teal, beta-2-microglobulin colored in gray, bound peptide colored in light grey. (A) Position 47 highlighted in light grey with above-median surface area yet low protrusion rank, (B) position 92 highlighted in light grey with above median surface area yet low protrusion rank, and (C) position 83 highlighted in light grey with below median surface area but high protrusion rank.

FIG. 12: Mean Snowflake (prior art; x axis) and Snowball (present invention; y axis) scores of antibody-verified Eplets based on HLA proteins carrying the respective Eplet. Error bars represent the respective score's standard deviations. The Figure shows the data without labels.

FIG. 13: Mean Snowflake (prior art; x axis; solvent accessibility) and Snowball (present invention; y axis; protrusion rank) scores of most exposed positions of each reported non-antibody-verified Eplet based on HLA proteins carrying the respective Eplet. The data is shown without labels.

FIG. 14: Solvent accessibility vs. protrusion rank of allele-specific positions of all reported Eplets. Mean Snowflake (solvent accessibility; x axis) and Snowball (protrusion rank; y axis) scores of all reported Eplets based on HLA proteins carrying the respective Eplet. Error bars represent the respective score's standard deviations. The figure shows the data without labels.

FIG. 15: AUCs for pooled CSA data of the PC considering various thresholds for Snowflake (x axis) and Snowball (y axis). A) 3D plot with AUC on z axis and greyscale B) Sum of median Snow scores in greyscale and on z axis C) Heatmap combining A and B. Snow sum values are depicted in greyscale, wherein if >35=white, and if 0=black; the pooled AUC is indicated in greyscale, wherein values above 0.48 are black. The Snowflake threshold is shown on the x-axis and Snowball threshold is shown on the y-axis. Peak AUCs were identified at a Snowflake threshold of 0.02 and Snowball threshold of 0.38. Linear models' AUCs have been calculated for Snowflake, Snow considering either of the thresholds being exceeded (SNF|SNB) and Snow considering both thresholds being exceeded simultaneously (SNF & SNB) for each locus individually and aggregated across loci (Table 1). Within each of the loci AUC remained identical or increased in the SNF & SNB model. In the pooled data, the AUCs of both the SNF|SNB and SNF & SNB models were slightly improved compared to the optimal Snowflake model.

FIG. 16: Akaike Information Criterion values (z axis) for Cox proportional hazard model considering models of (A) log(Snow+1), (B) log(Snow+1) and log(PIRCHEc+1), and (C) log(Snow+1) and log(PIRCHEc+1) and A-B-DR scores at varying Snowflake (x-axis) and Snowball (y-axis) thresholds. The respective minimum AICs are 1206008 (A, Snowflake=0.00, Snowball 0.00), 1205923 (B, Snowflake=0.30, Snowball=0.68) and 1205753 (C, Snowflake=0.30, Snowball=0.68). The heatmap in D) indicates the minimum AICs of the respective models (of each circle shown: the outer ring represents Snow, the middle ring Snow and PIRCHEc, the inner ring Snow, PIRCHEc and A-B-DR scores) in the same grey range (0=white, 1.6=black) of log-transformed AICs to indicate optimal thresholds for Snowflake (x-axis) and Snowball (y-axis). Circles indicate optimal thresholds (lowest AIC) considering the respective model.

FIG. 17: AUCs for pooled CSA data of the PC considering various thresholds for Snowflake (x axis) and Snowball (y axis) with Snow being configured as either exceeding Snowflake thresholds or Snowball thresholds or both. A) 3D plot with AUC on z axis and greyscale B) Sum of median Snow scores in greyscale and on z axis C) Heatmap combining A and B using the same greyscale (Snow sum of 60=white, 0=black). Peak AUCs for individual loci were identified and labeled accordingly. Snowflake threshold (x-axis) and Snowball threshold (y-axis). For the pooled CSA, an optimal AUC was reached at a Snowflake threshold of 0.07 and Snowball threshold of 0.62.

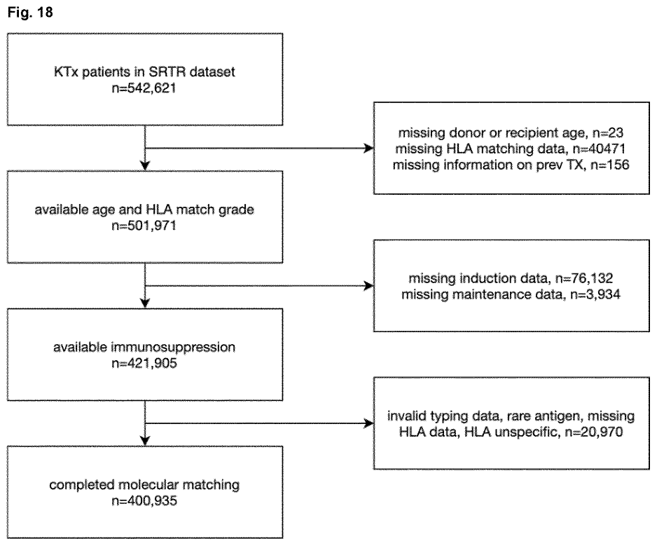

FIG. 18: The subset of the SRTR kidney transplant dataset considered for statistical models comprised 400,935 kidney transplantations.

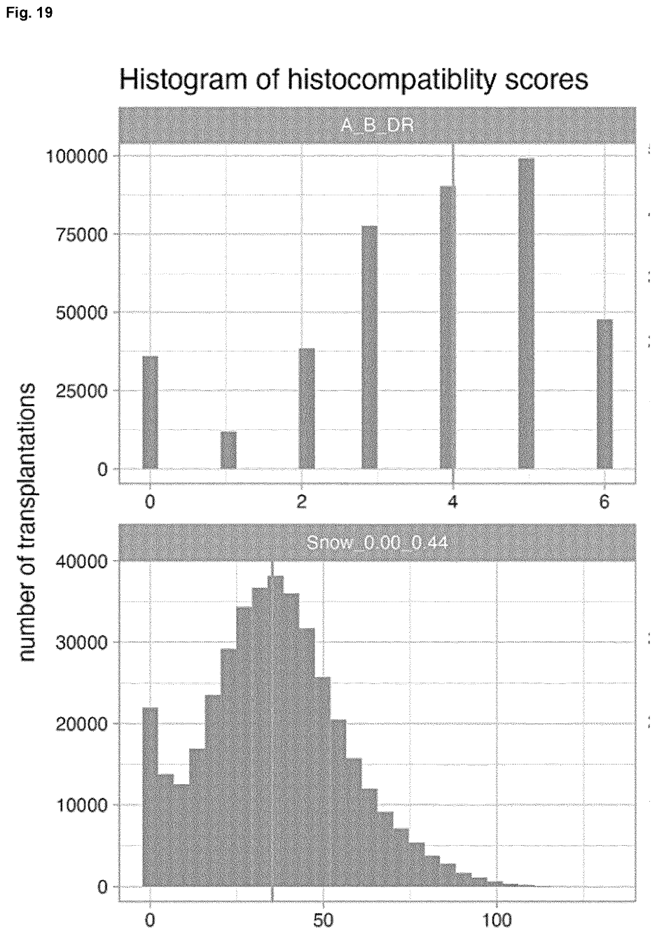

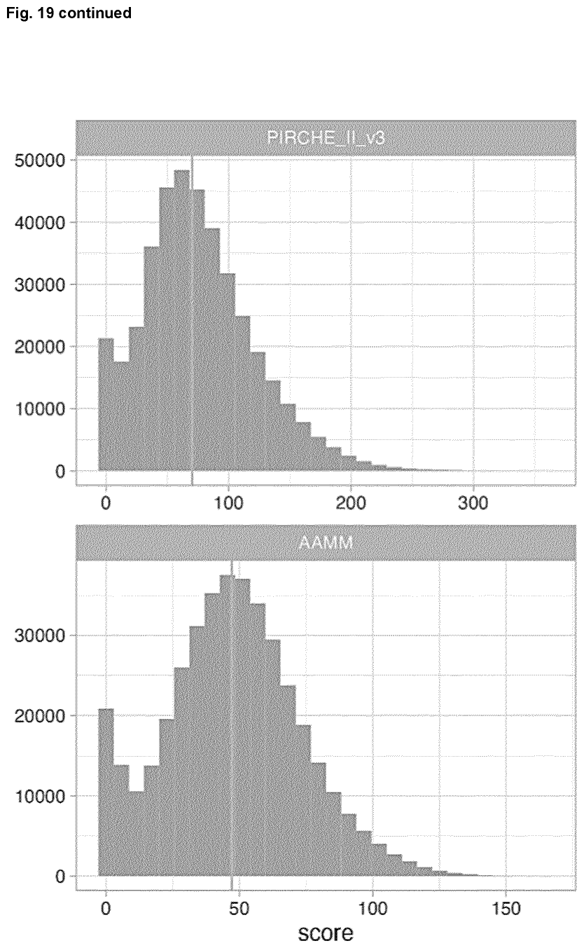

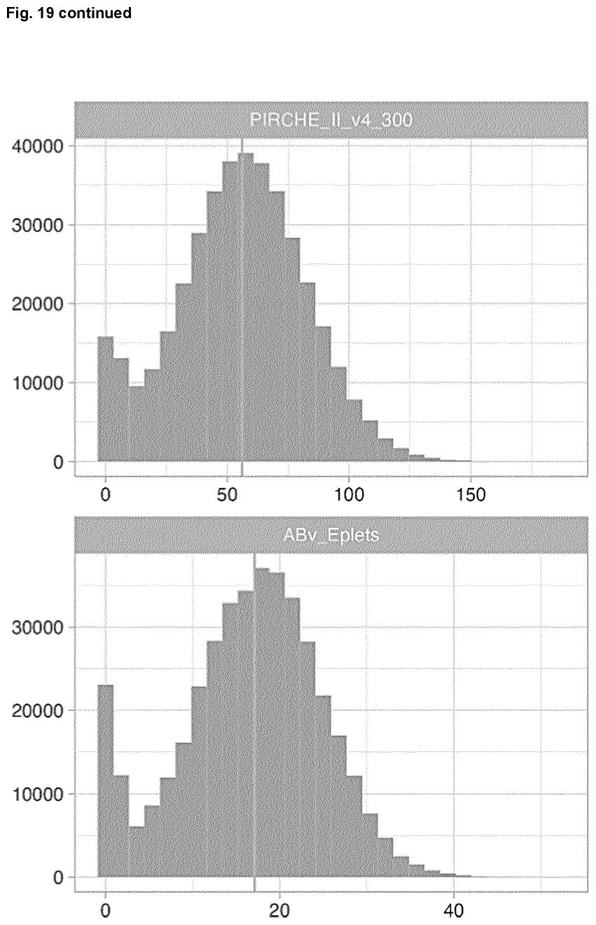

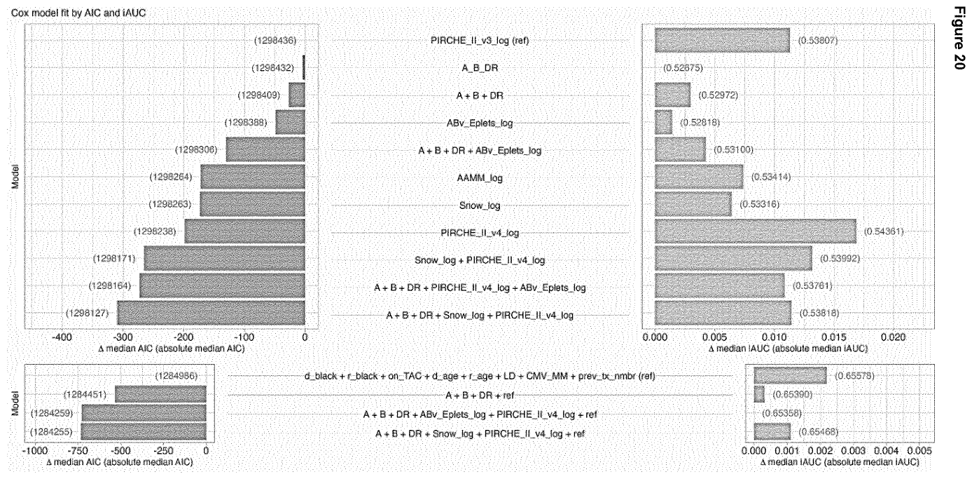

FIG. 19: Histograms of the considered histocompatibility metrics reveal a comparable distribution pattern across all, albeit with very different numeric ranges.

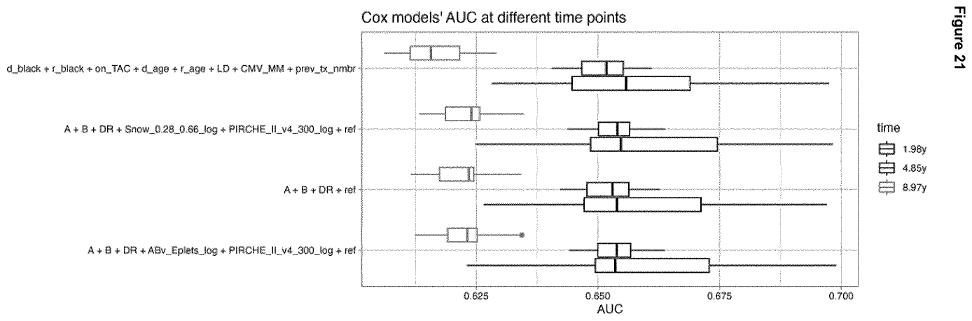

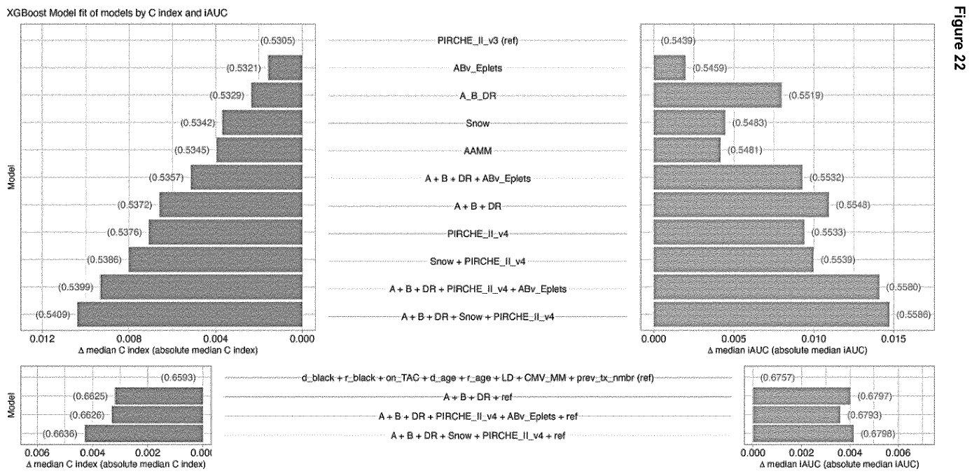

FIG. 20: Butterfly plot of model performance of Cox models in terms of AIC (left, lower is better) and iAUC (right, higher is better). Top panel considers models only consisting of histocompatibility metrics, bottom panel considers models of known clinical risk factors in conjunction with histocompatibility metrics. Bars denote the delta compared to the respective reference model (i.e. PIRCHE version 3 for top panel; donor or patient being black, Tacrolimus maintenance immunosuppression, donor and patient age, living donor, CMV mismatch, and number of prior transplantations for bottom panel). Details of the selected Cox proportional models with their respective hazard ratios are presented in Table 5.

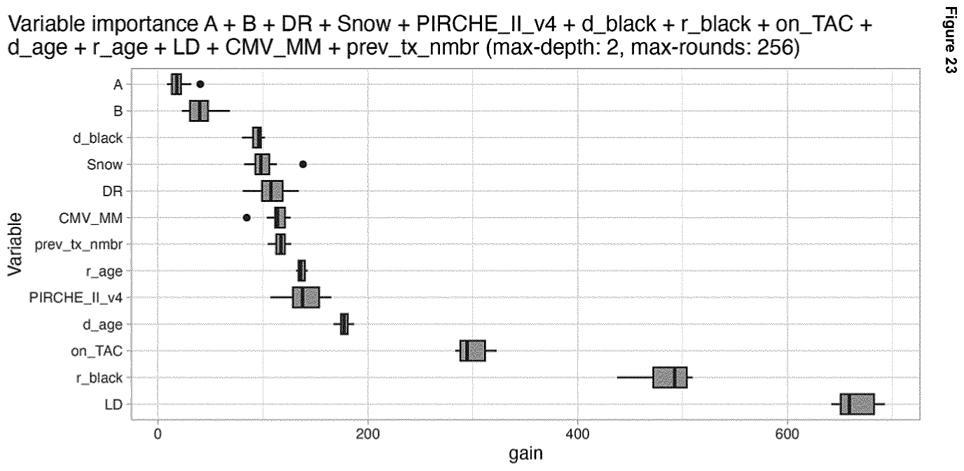

FIG. 21: Distribution of repeated Cox models' AUC at 1.98 years, 4.85 years and 8.97 years (corresponding to the 25th, 50th and 75th percentiles of the observation period) indicate higher performance of the histocompatibility-augmented models in long-term prediction.

FIG. 22: Butterfly plot of model performance in terms of C index (left, larger is better) and iAUC (right, larger is better). Top panel considers models only consisting of histocompatibility metrics, bottom panel considers models of known clinical risk factors in conjunction with histocompatibility metrics. Bars denote the delta compared to the respective reference model (i.e. PIRCHE version 3 for top panel; donor or patient being black, Tacrolimus maintenance immunosuppression, donor and patient age, living donor, CMV mismatch, and number of prior transplantations for bottom panel).

FIG. 23: Variable importance based on gain metric (higher is better) of XGBoost models considering HLA-A (least important), -B, -DR matching, Snow, PIRCHE-II (PIRCHE_II_v4), donor being black (d_black), recipient being black (r black), Tacrolimus maintenance (on_TAC), donor age (d_age), recipient age (r_age), living donor transplantation (LD, most important), CMV mismatch (CMV_MM) and number of previous transplantations (prev_tx_nmbr).

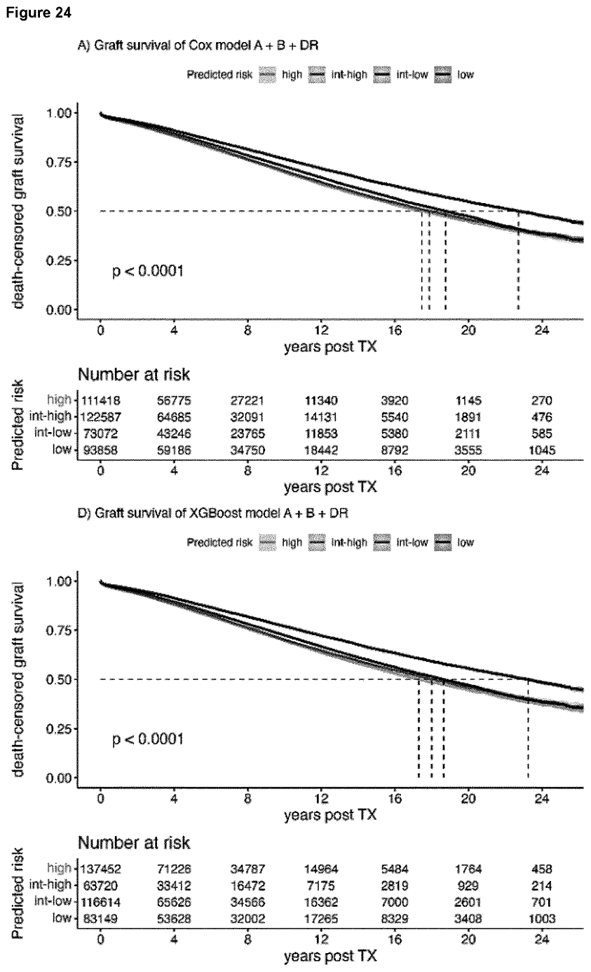

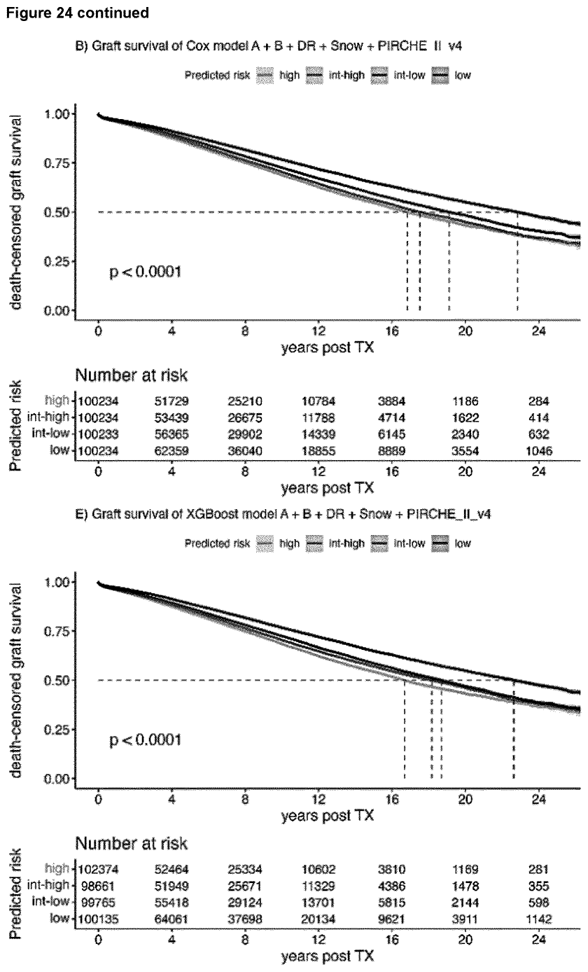

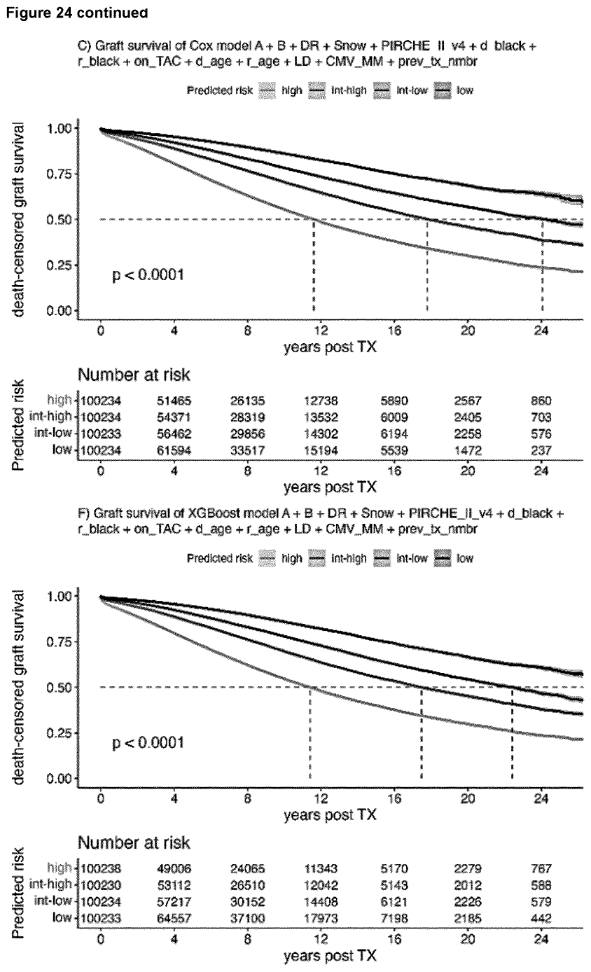

FIG. 24: Kaplan Meier plots of selected Cox and XGBoost models predicting risk of early graft loss. A) Cox model of HLA-A, -B, and -DR matching, B) Cox model of HLA-A, -B, -DR, Snow and PIRCHE matching, C) Cox model of HLA-A, -B, -DR, Snow and PIRCHE matching, donor being black black, recipient being black, Tacrolimus maintenance, donor age, recipient age, living donor, CMV match, and number of previous transplantations, D) XGBoost model of HLA-A, -B, and -DR matching, E) XGBoost model of HLA-A, -B, -DR, Snow and PIRCHE matching, F) XGBoost model of HLA-A, -B, -DR, Snow and PIRCHE matching, donor being black black, recipient being black, Tacrolimus maintenance, donor age, recipient age, living donor, CMV match, and number of previous transplantations.

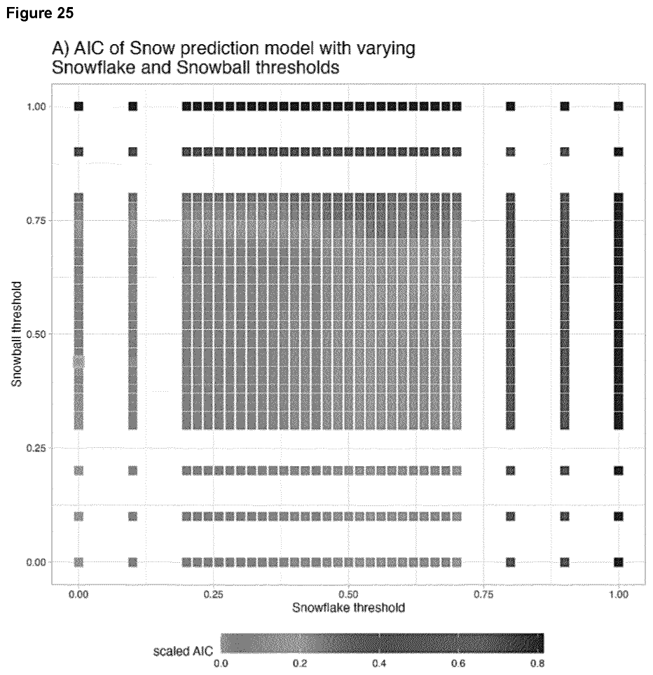

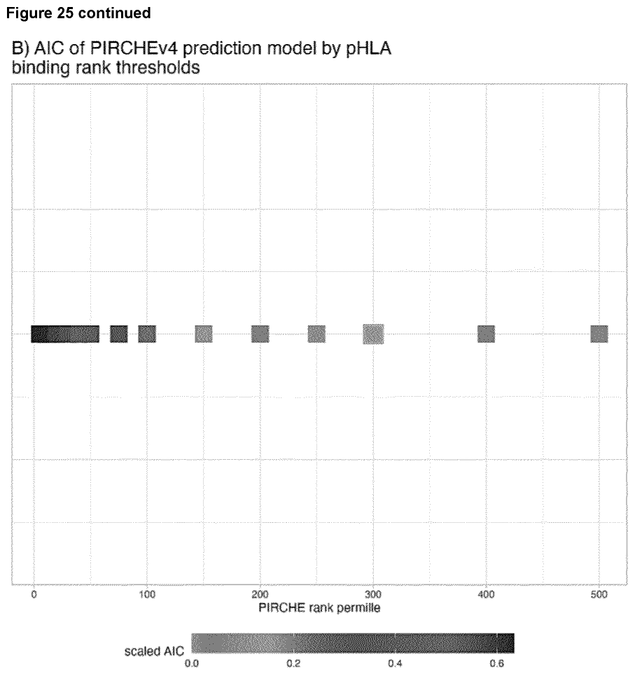

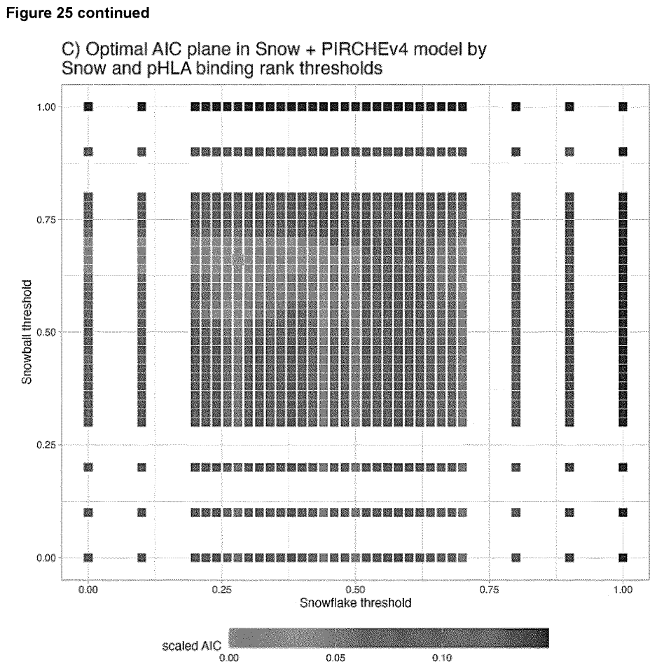

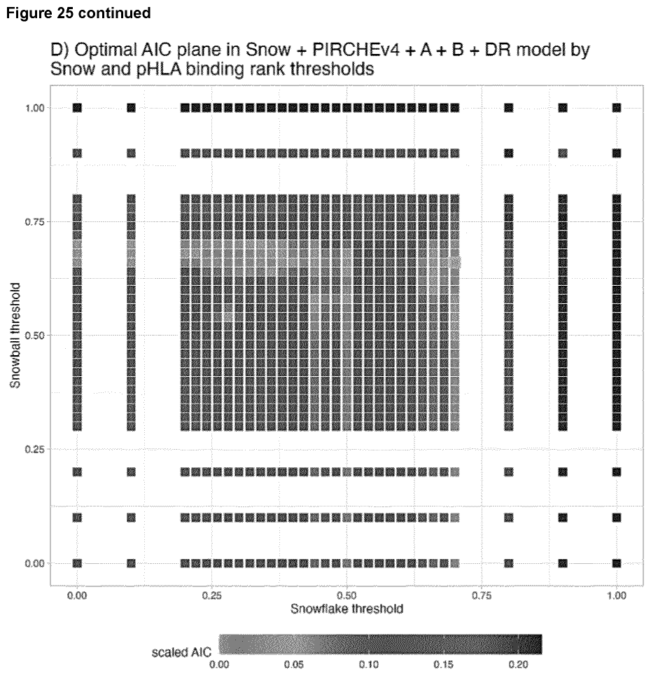

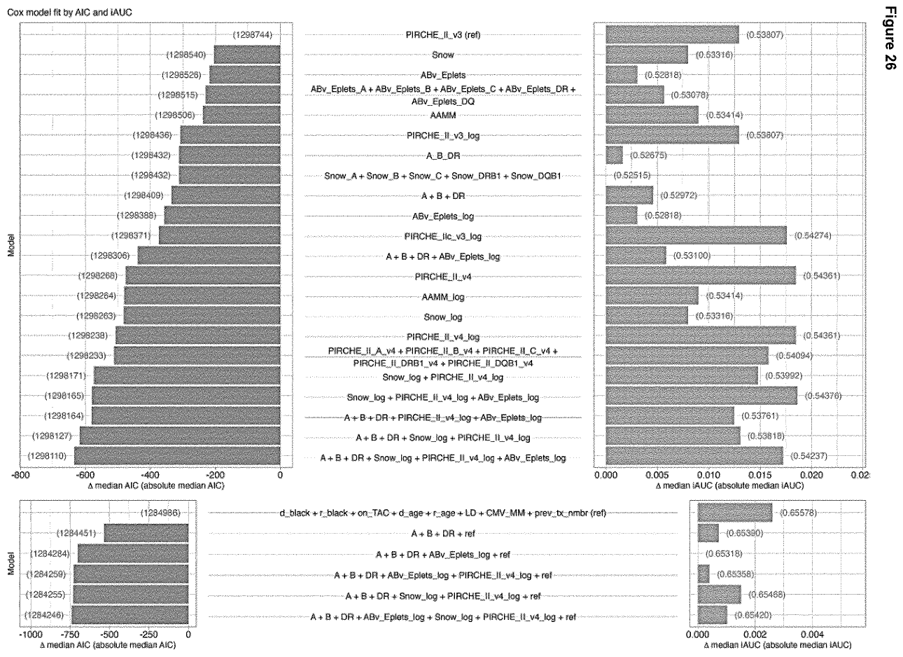

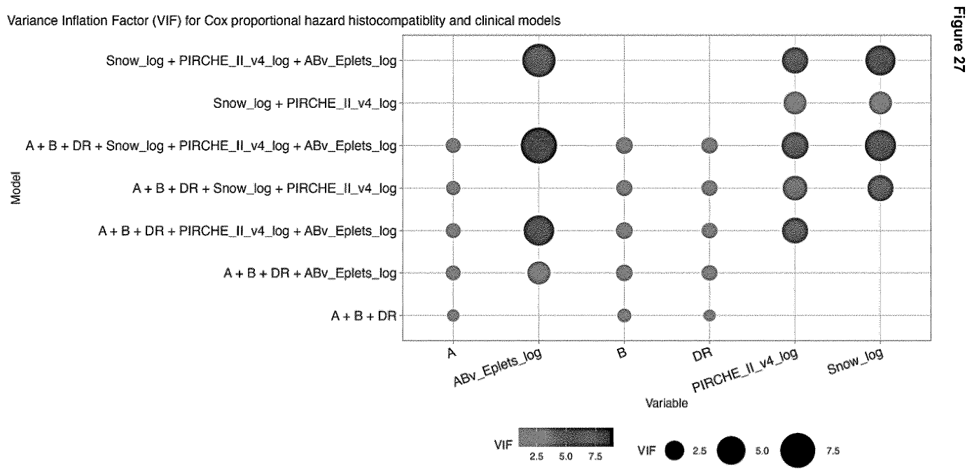

FIG. 25: Identifying thresholds for optimal AIC in Snow and PIRCHE models considering death-censored kidney graft survival in the SRTRC. Colors indicate the respective model's scaled AIC, with yellow indicating lower (better) values and purple indicating higher (worse) values. A) Optimal AIC (1911575) for a univariable Cox model considering only the log-transformed Snow score was reached at threshold pairs 0.00/0.44 (light grey border/box). B) Optimal AIC (1911524) for a univariable Cox model considering only the log-transformed PIRCHE score was reached at a threshold of 300‰ (light grey border/box). C) In a multivariable Cox model of Snow and PIRCHE, the optimal thresholds shifted towards a more restrictive 0.28/0.66/300 (light grey border/box), yielding an AIC of 1911438. D) Adding HLA-A, —B and -DR matching to the multivariable Cox model shifted the optimal AIC (1911373) towards 0.70/0.66/300 (light grey border/box). However, the configuration of 0.28/0.66/300 has a very similar performance with an AIC of 1911376.

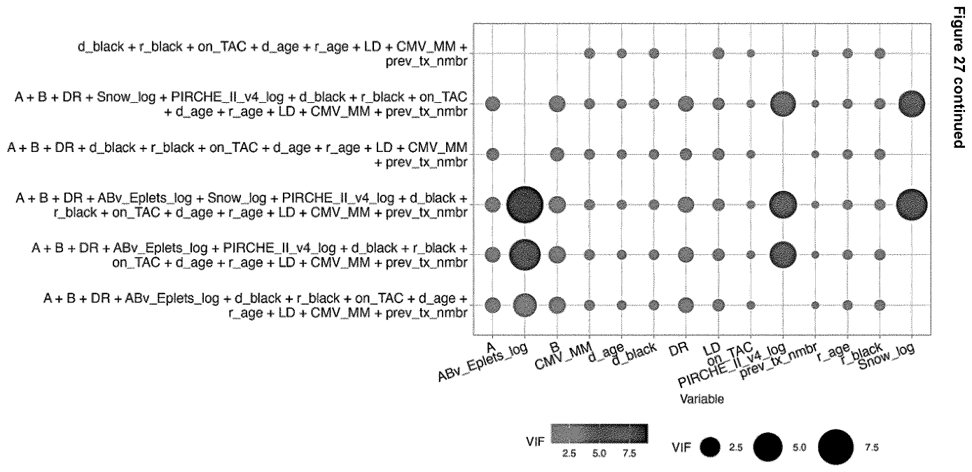

FIG. 26: Extended butterfly plot of AIC and iAUC of Cox proportional hazards models predicting graft survival.

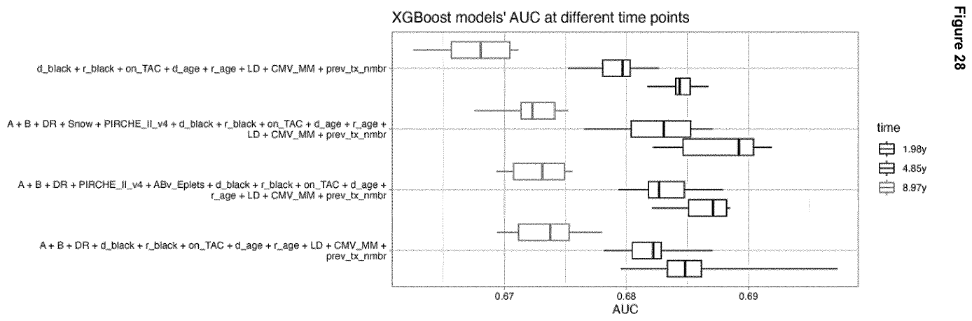

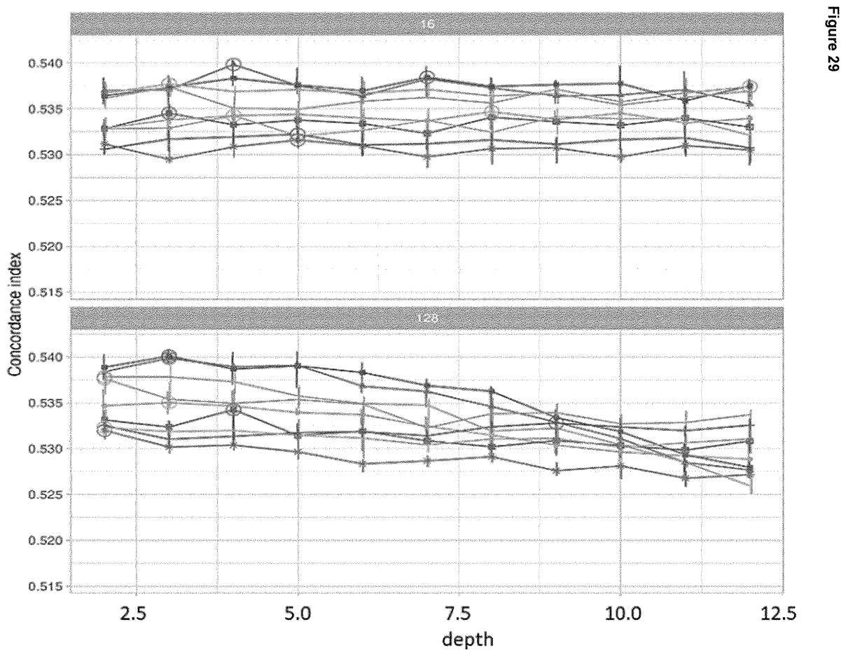

FIG. 27: Cox variance inflation factors (VIF) for multivariable prediction models. In particular for models containing both Eplets and Snow, elevated VIF can be observed, suggesting these variables are codependent.

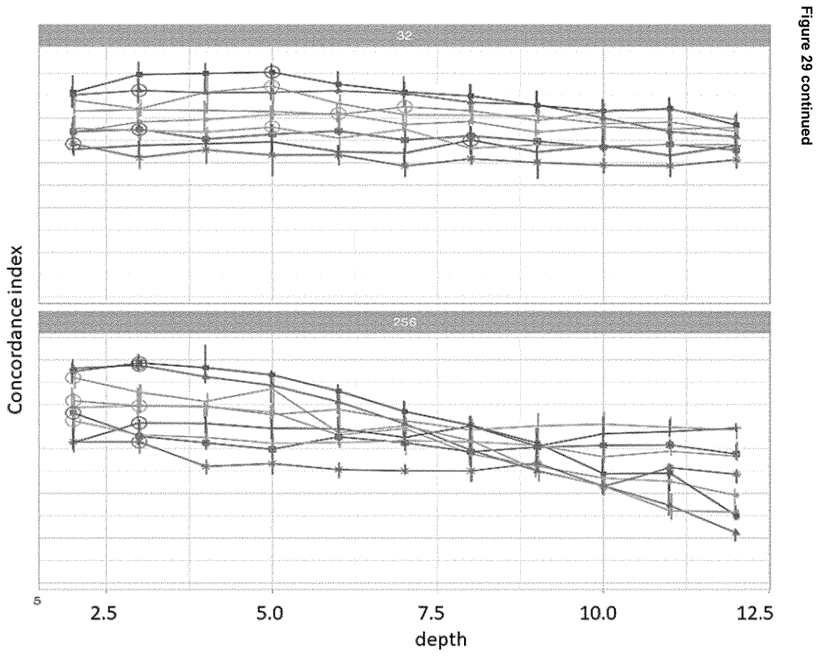

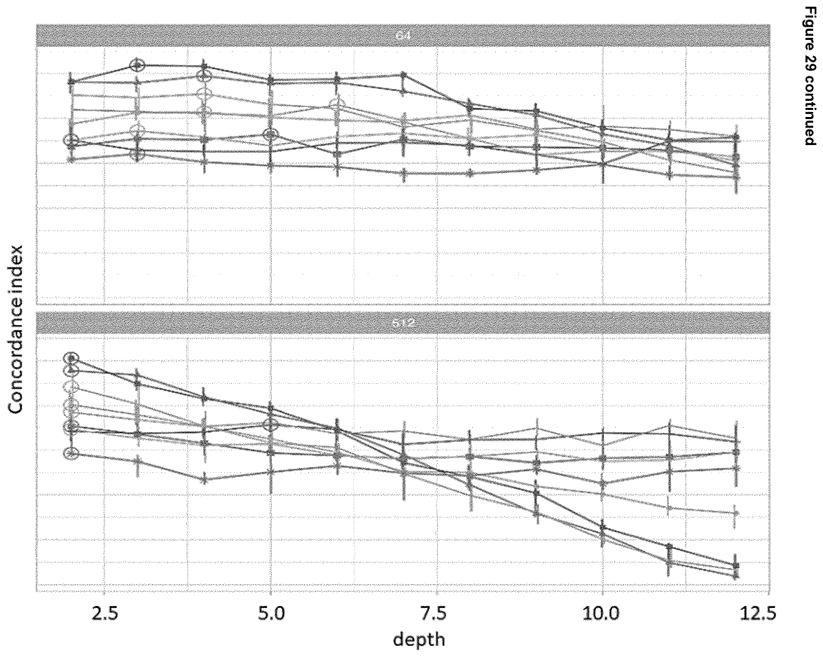

FIG. 28: Distribution of repeated XGBoost models' AUC at 1.98 years, 4.85 years and 8.97 years (corresponding to the 25th, 50th and 75th percentiles of the observation period) indicate higher performance of the histocompatibility-augmented models all time points.

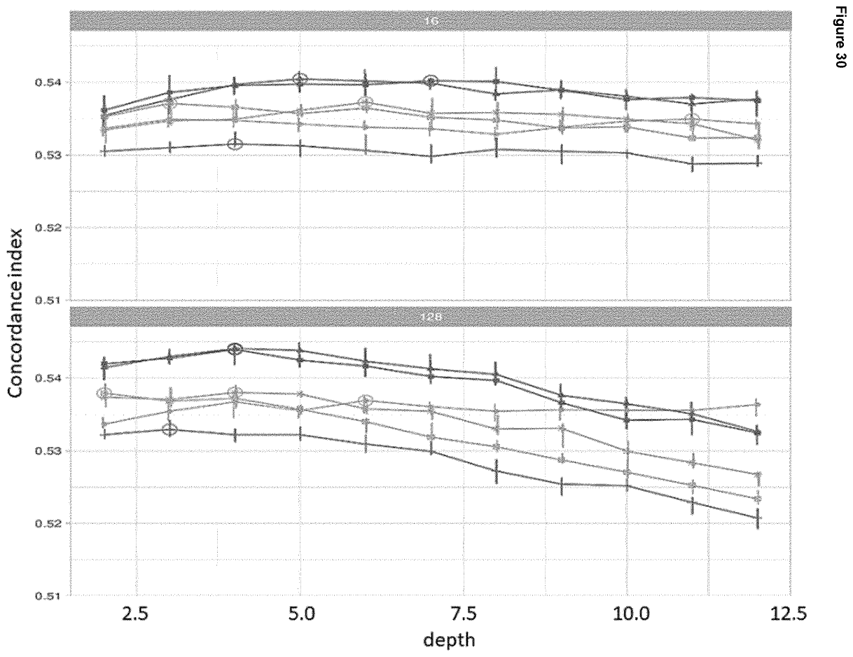

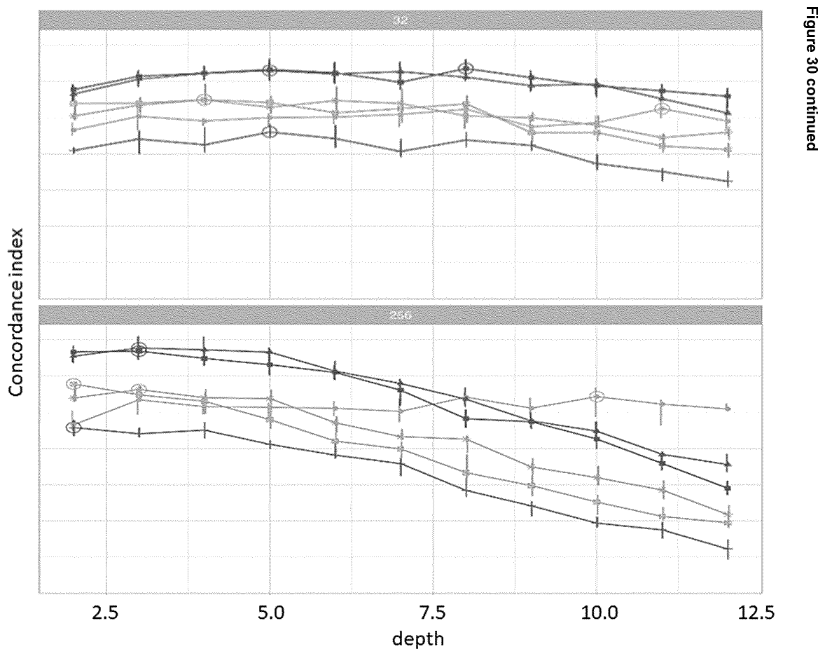

FIG. 29: Harrel's concordance index (y axis) of repeated XGBoost models of molecular matching sum scores considering maximum model depth (x axis) and number of trees per model (panels). Points indicate the respective median C index with the error bars indicating the first and third quartiles. Circles around points indicate the respective models maximum value per panel. The highest ranking data are the models combining ‘A+B+DR+PIRCHE II v4+ABv Eplets’, and ‘A+B+DR+Snow+PIRCHE II v4’. The labelling of the X-axis and Y-axis is consistent for all panels (graphs) of the figure.

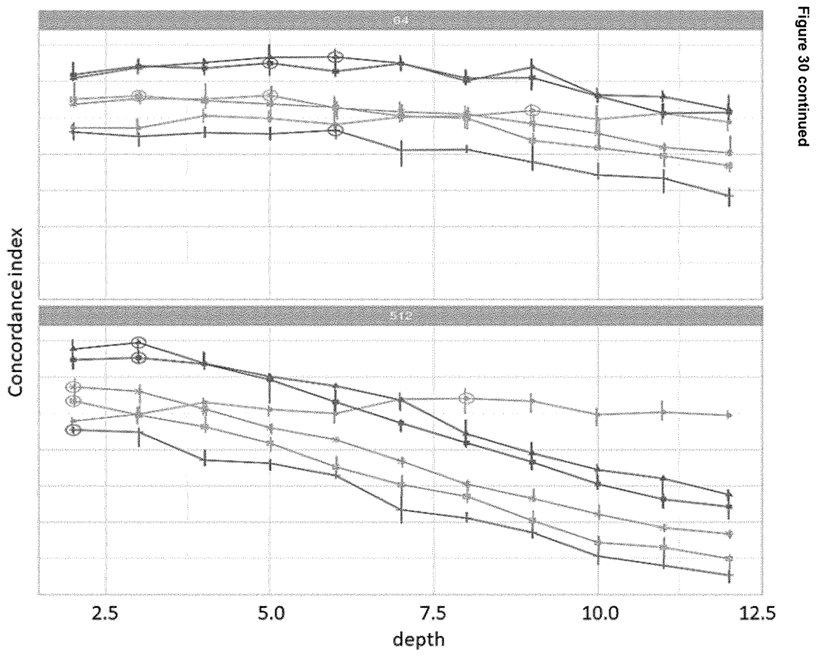

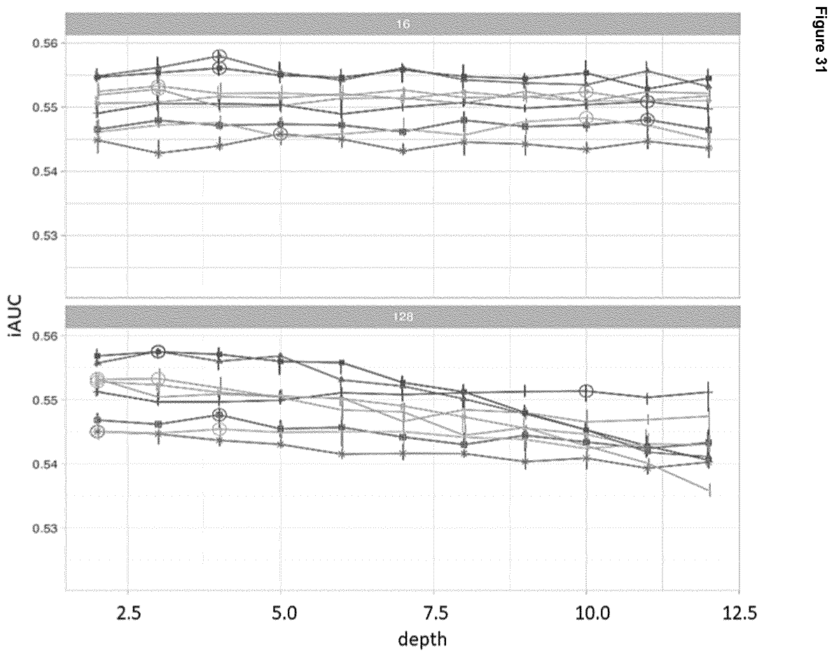

FIG. 30: Concordance index (y axis) of repeated XGBoost models of molecular matching scores per locus considering maximum model depth (x axis) and number of trees per model (panels). Points indicate the respective median C index with the error bars indicating the first and third quartiles. Circles around points indicate the respective models maximum value per panel. The highest ranking data are the models combining ‘A+B+DR+PIRCHE II A v4+PIRCHE II B v4+PIRCHE II Cv4+PIRCHE II DRB1+PIRCHE II DQB1+ABv Eplets A+ABv Eplets B+ABv Eplets C+ABv Eplets DR+ABv Eplets DQ’, and ‘A+B+DR+PIRCHE II A v4+PIRCHE II Bv4+PIRCHE II Cv4+PIRCHE II DRB1+PIRCHE II DQB1+Snow A+Snow B+Snow C+Snow DRB1+Snow DQB1’. The labelling of the X-axis and Y-axis is consistent for all panels (graphs) of the figure.

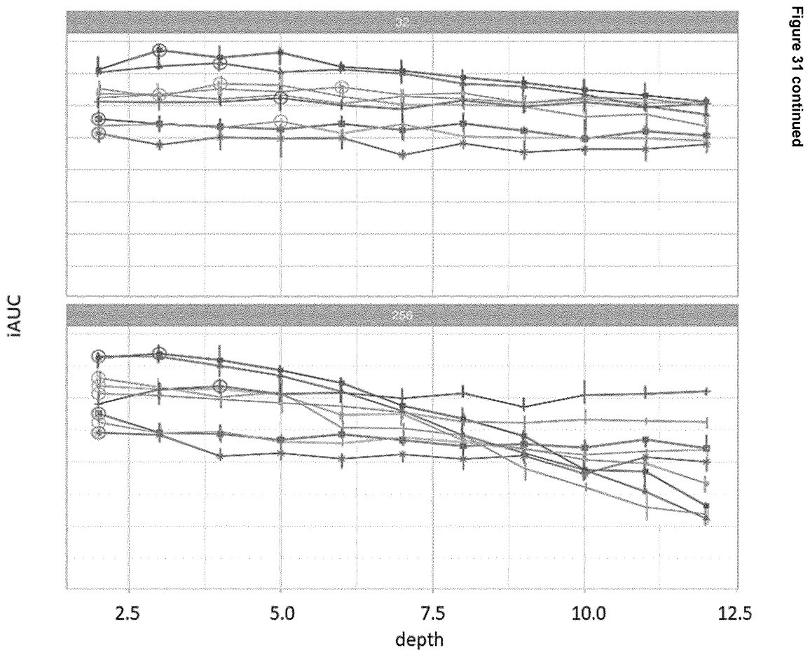

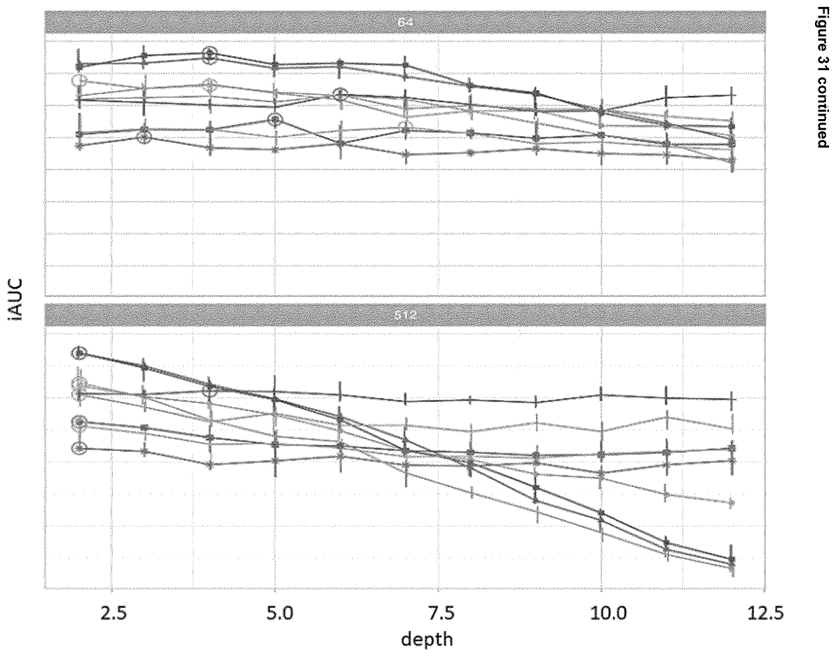

FIG. 31: Integrated AUC (y axis) of repeated XGBoost models of molecular matching sum scores considering maximum model depth (x axis) and number of trees per model (panels). Points indicate the respective median C index with the error bars indicating the first and third quartiles. Circles around points indicate the respective models maximum value per panel. The highest ranking data are the models combining ‘A+B+DR+PIRCHE II v4+ABv Eplets’, and ‘A+B+DR+Snow+PIRCHE II v4’. The labelling of the X-axis and Y-axis is consistent for all panels (graphs) of the figure.

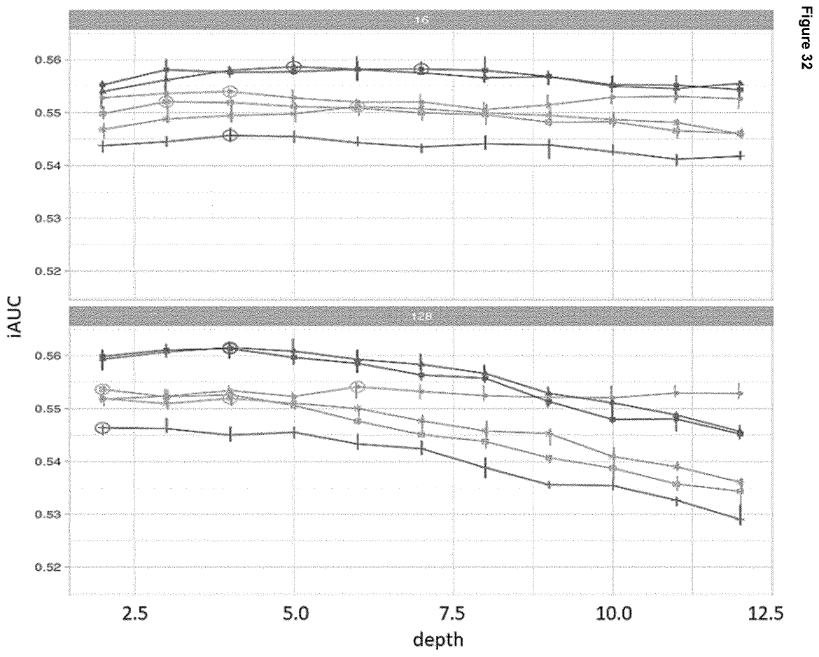

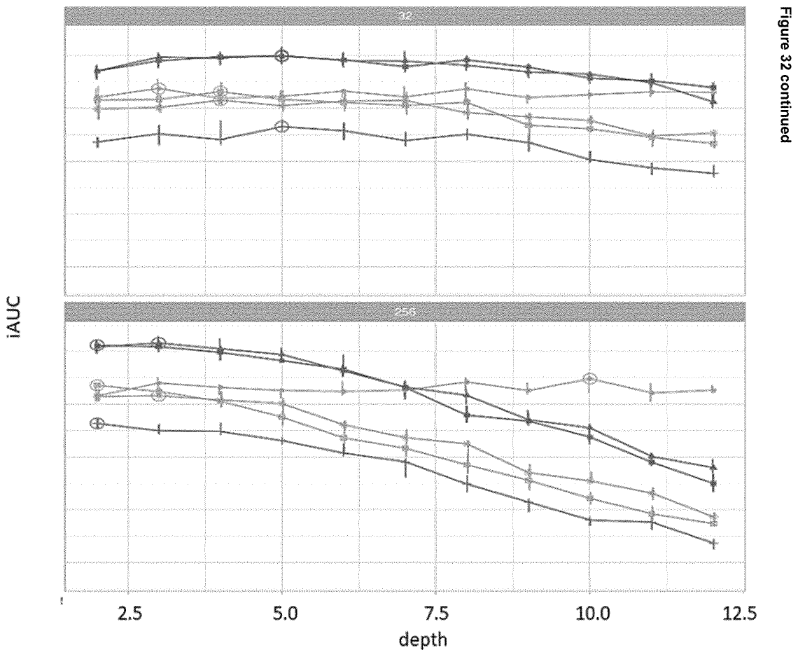

FIG. 32: Integrated AUC (y axis) of repeated XGBoost models of molecular matching scores per locus considering maximum model depth (x axis) and number of trees per model (panels). Points indicate the respective median C index with the error bars indicating the first and third quartiles. Circles around points indicate the respective models maximum value per panel. The highest ranking data are the models combining ‘A+B+DR+PIRCHE II A v4+PIRCHE II B v4+PIRCHE II Cv4+PIRCHE II DRB1+PIRCHE II DQB1+ABv Eplets A+ABv Eplets B+ABv Eplets C+ABv Eplets DR+ABv Eplets DQ’, and ‘A+B+DR+PIRCHE II A v4+PIRCHE II Bv4+PIRCHE II Cv4+PIRCHE II DRB1+PIRCHE II DQB1+Snow A+Snow B+Snow C+Snow DRB1+Snow DQB1’. The labelling of the X-axis and Y-axis is consistent for all panels (graphs) of the figure.

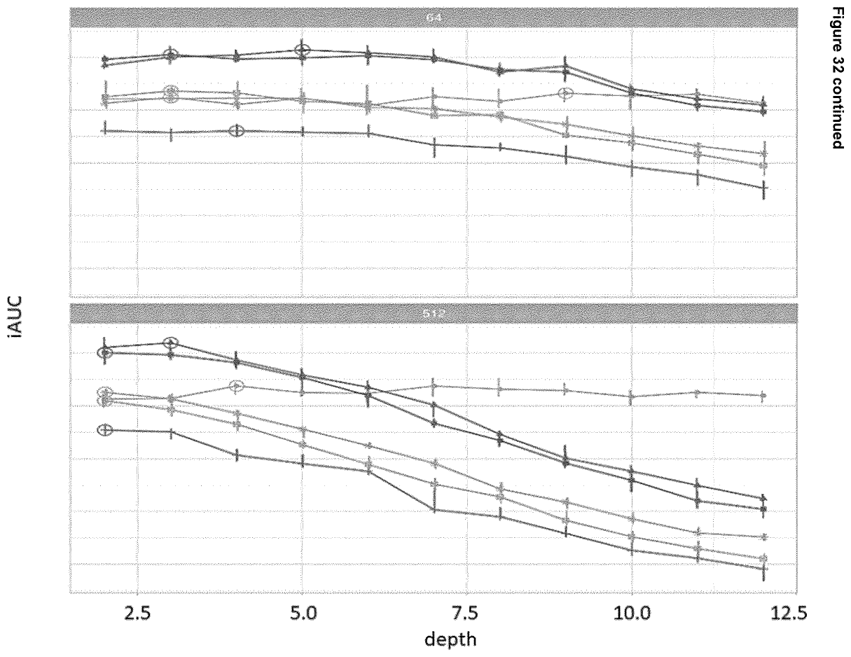

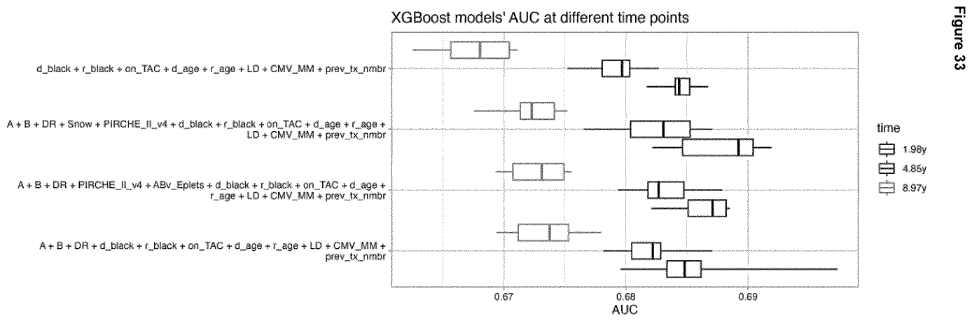

FIG. 33: Distribution of repeated XGBoost models' AUC at 1.98 years, 4.85 years and 8.97 years (corresponding to the 25th, 50th and 75th percentiles of the observation period) indicate higher performance of the histocompatibility-augmented models at all tested time points with a slight advantage of molecular matching-containing models in short- and mid-term prediction.

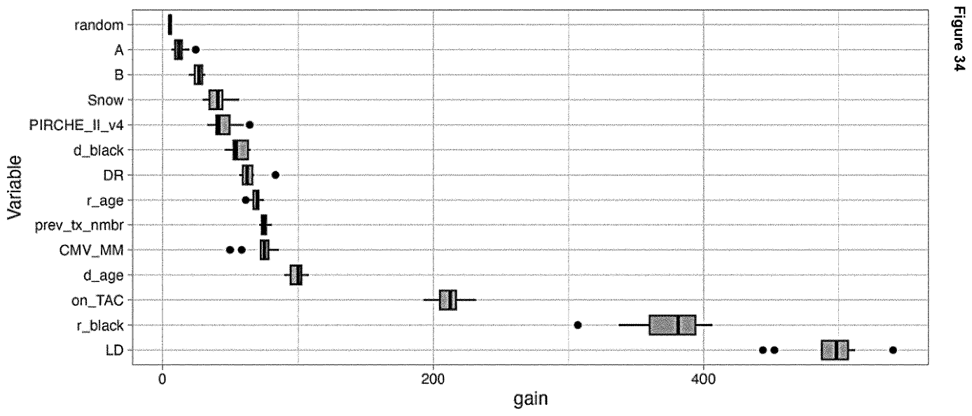

FIG. 34: Variable importance based on gain metric (higher is better) of XGBoost model considering HLA-A (least important), -B, -DR matching, Snow, PIRCHE-II (PIRCHE_II_v4), donor being black (d_black), recipient being black (r black), Tacrolimus maintenance (on_TAC), donor age (d_age), recipient age (r_age), living donor transplantation (LD, most important), CMV mismatch (CMV_MM), number of previous transplantations (prev_tx_nmbr) and a random variable. Even though HLA-A matching is of low overall importance, it still outperforms a variable of random values artificially included into the model.

### EXAMPLES

The invention is further described by the following examples. These are not intended to limit the scope of the invention but represent preferred embodiments of aspects of the invention provided for greater illustration of the invention described herein.

Herein, the inventors developed a method that repeatedly applies ellipsoid-fitting and protrusion-ranking to adapt the concept to non-elliptic protein structures. Each iteration of ellipsoid-fitting considers a subset of nearby atoms and applies majority-voting to aggregate individual ellipsoids' rankings.

Based on the reported finding by Niemann et al. that HLA proteins amino acids' surface area is dependent on individual HLA alleles' structure (the so called “Snowflake” algorithm), [8] the inventors extended the prediction pipeline to HLA-DRB1 and -DQB1, and integrated the algorithm described herein, which allowed to consider surface area and localized protrusion rank in a combined HLA protein-specific amino acid mismatch algorithm, (herein also referred to as “Snow” algorithm).

To evaluate the predictive performance of Snow with immunological events, we reanalyzed the previously reported dataset of 231 pregnancies from the University Hospital Basel and the publicly available dataset of kidney transplants from the Scientific Registry of Transplant Recipients (SRTR).

### Example 1

Extraction of Experimental Structures from PDB

HLA protein structures were extracted from the PDB using the full text search term “hla”. The found structures were filtered by structures comprising chains “A”, “B” and “C”, whereas for HLA Class I chain B's amino-acid sequence was expected to match beta-2-microglobulin (UniprotKB entry P61769), for HLA-DR chain A's amino-acid sequence was expected to match DRA1*01:01, and for HLA-DQ chain A's amino-acid sequence was expected to match a known DQA1. The supposed alpha and beta chains were aligned to the IPD/IMGT-HLA database [31](version 3.47.0) using BLAST [32] via the Biopython library [33]. HLA-allele names and identifiers were assigned to the protein structures based on the maximum number of identical amino acid configurations and lowest HLA identifier in case of a tie.

### Structural Prediction of Non-Crystallized HLA Proteins

As previously reported, the AlphaFold [34-36](DeepMind Technologies Limited, London, UK) protein structure inference pipeline (v2.0) was applied to supplement the PDB data. [8] The AlphaFold software was installed on an Amazon Web Services Elastic Cloud Compute server (Amazon Web Services Inc., Seattle, US) to run de novo folding. HLA proteins were selected for structure prediction when their serologic and antigenic groups were not already covered by structures from the PDB. A total of 78 structures were predicted (HLA-A: 14, HLA-B: 16, HLA-C: 7, HLA-DRB1: 9, HLA-DQA1/-DQB1: 6×5=30).

### Render Peptide in Non-Redicted Structures' Binding Groove

As previously reported [8], inferred HLA Class I structures were supplemented with bound peptides by considering known binders from the Immune Epitope Database (IEDB, www.iedb.org) [37] and docking these through the Anchored Peptide-MHC Ensemble Generator (APE-Gen). [38] Structural docking prediction for HLA Class II is however less reliable. The docking framework Dockit was evaluated but structural predictions could not reproduce peptide alignment in experimental structures sufficiently well to use it for docking prediction in inferred structures (data not shown). Instead, the top 5 inferred options per predicted structure were superimposed as described by Golub et al [39] via the Biopython library [33] to experimental structures based on the Ca atoms of four amino acids at the outer ends of the binding groove helices (HLA-DRA1/-DRB1: A:68, A:57, B:60, B:81; HLA-DQA1/-DQB1: A:71, A:60, B:56, B:85). Peptides of available experimental structures (HLA-DRA1/-DRB1: 18, HLA-DQA1/-DQB1: 18) were copied into the inferred structure for further analyses without additional energy optimization.

### Repeated Ellipsoid Protrusion Rank

Coordinates of non-hydrogen atoms of all alpha, beta and peptide amino acids were considered in ellipsoid analysis. Iterating over each atom as the current center, all nearby atoms within an Euclidean distance w were selected. For these atoms, after regularization, an ellipsoid was fitted and geometrically centered considering linear least squares [40]. Lacking a general formula describing ellipsoids, the fitted ellipsoid and its atom coordinates were transformed affine into a sphere [41]. Distance between sphere center and transformed atom coordinate was ranked, corresponding to protrusion of fitted ellipsoid. For each atom of a given structure, median protrusion ranks within all fitted ellipsoids were calculated and stored. The maximum median protrusion rank of each atom of a residue was considered as residue-specific protrusion rank (Snowball score) per HLA protein. The process is shown in FIG. 3.

Final models considered a w of 15 Å (i.e., area of a circle of ~700 Å²), which corresponds to reports of the antibody binding site stretching across an area of 30 Å×20 Å or 750 Å² [42, 43].

### Neural Network Design

The experimental and inferred HLA structures comprise only a fraction of known HLA proteins. To create a database of protruding residues for all HLA proteins, long short-term memory bidirectional recurrent neural networks (BRNN) [44] were implemented, that chained the HLA alpha chains' (HLA Class I) and HLA beta chains' (HLA Class II) amino-acid configurations as one-hot-encoded inputs and the residues' protrusion ranks as output. The network configuration stacked a bidirectional layer with rectified linear unit (ReLU) activation of 128 neurons, a second bidirectional layer with 64 ReLU neurons, a third bidirectional layer with 32 ReLU neurons and the output layer considering a linear activation function. Network training used Adam optimization for efficient gradient descent [45]. The model-generation and inference was implemented in the Python programming language (Python Software Foundation, version 3.5.1) using Keras (https://keras.io)/Tensorflow 2 (https://www.tensorflow.org). Three independent networks were trained for HLA Class I, HLA-DRB1 and HLA-DQA1/-DQB1 respectively. The overall data flow of the Snowball prediction pipeline is depicted in FIG. 4.

### Considering Ellipsoid Protrusion and Surface Area in HLA Amino Acid Matching

The recently proposed Snowflake algorithm considers HLA protein-specific solvent accessibility of amino acids. Mismatched donor amino acids exceeding a solvent accessibility threshold are considered antibody accessible, informing risk for immune response [8]. Following this concept, the present algorithm (Snowball algorithm) was integrated, forming the “Snow” algorithm. The number of donor amino acid mismatches that are both exceeding a solvent accessibility threshold and exceeding an amino acid protrusion threshold are hypothesized to increase immunological risk in a transplant setting.

### Reference Epitope Definitions

Eplet definitions of the HLA Epitope Registry (www.epregistry.com.br, version 3.0) have been considered for identifying Eplets' Snowflake and Snowball scores [46].

### Statistical Analysis

Correlation analyses between the prior art methods ElliPro and Snowflake and the present method (Snowball) were performed by calculating the Spearman's rank correlation coefficient (spearmans rho).

All calculations were executed in R software (R 3.6.1, R Foundation for Statistical Computing, Vienna, Austria).

### Results

Repeated Local Protrusion (Snowball) Vs. Global Protrusion (ElliPro)

Repeated local protrusion strongly correlates with global protrusion ranks calculated by ElliPro (r=0.6508) considering A*02:01 (PDBID 5HHQ, FIG. 5). Average protrusion ranks of the present method (Snowball) were predicted to be higher than with the state of the art method ElliPro, which can be explained by averaging multiple fitted ellipsoids, of which some are centered far away from the respective amino acid.

Varying sphere radii ω indicate the sensitivity of the present method (Snowball) towards small structural changes. It can be observed that with large ω, the calculated protrusion ranks converge to calculations of ElliPro. Conversely, very small ω correspond to almost constantly high protrusion ranks. Interestingly, the suggested w of 15 Å allows discrimination of amino acids' protrusion in the helices (FIG. 6).

### Position-Specific Protrusion Variance

Considering the Snowball scores for a reference set of alleles as defined by Matern et al [14] indicates some variation of position-specific protrusion at certain amino acid positions.

The Present Method Vs. The Prior Art Method “Snowflake”

Across all HLA loci, Snowball (present invention) mean and median scores were higher than respective Snowflake (prior art method) scores. However, a strong correlation between Snowball and Snowflake can be observed with Spearman's rho above 0.8 (FIG. 8). Root mean square difference of position-specific pairwise protrusion within the most frequent HLA experimental structures reported in the PDB is slightly lower than inter-allele comparisons for some alleles.

### Eplets' Snowflake and Snowball

Solvent accessibility and protrusion of exposed amino acid positions within antibody-verified Eplets were plotted in FIG. 10. A scatter plot of all antibody-verified Eplet-positions' Snowflake and Snowball scores is provided in FIG. 10. Corresponding graphs for non-antibody verified Eplets are provided in FIGS. 13 and 14.

### Discussion

The herein presented “Snowball” prediction model for repeated local protrusion estimation which has been specifically designed for HLA proteins and integrated it with the previously developed Snowflake model into the combined Snow matching algorithm. Fitting global ellipsoids and defining residues' protrusion ranks for protein structures has been suggested by Thornton et al [12,13] and implemented by Ponomarenko et al [11] before. Opposed to that, the present method repeatedly fits ellipsoids for sub-structures of the whole molecule to better adapt to smaller protrusion changes in large proteins. Averaging multiple fitted ellipsoids' protrusion ranks compensates for mispredictions due to atoms located in the outer search space. The inventors developed a prediction pipeline to extrapolate repeated local protrusion ranks for HLA proteins without available experimental data. To that extent the present method improves the previously suggested Snowflake prediction pipeline to also cover HLA Class II proteins.

The herein presented data shows a strong correlation between the present method (Snowball) and the prior art ElliPro data (FIG. 5). However, specifically in the characteristic helix structures of the HLA binding groove, the present method (Snowball) is more sensitive than the prior art ElliPro approach, allowing to better distinguish inward facing amino acids and outbound amino acids (FIG. 6). We identified a radius w of 15 Å as beneficial for individual ellipsoids search space, corresponding to reported antibody binding sites [42, 43]. The characteristic shape of HLA proteins is thus better described by repeated local ellipsoid fitting than by global ellipsoids. Although not tested herein, the applied method is also applicable to other proteins than HLA. In particular for proteins folding into non-elliptic structures, this method may be beneficial in protrusion estimation.

Across different HLA alleles protrusion scores vary per amino acid position. In particular in the HLA binding grooves helical structures, Snowball scores are spread wider compared to conserved positions of the structures' backbones.

The Snowball score is very strongly correlated with Snowflake's solvent accessibility prediction (FIG. 8), however not being identical. Protrusion and surface area distributions Eplets were provided (FIG. 10) promoting further research in identification of Eplet immunogenicity. In FIG. 11, amino acid positions with contradicting Snowball and Snowflake scores were highlighted. Considering the larger structure by local ellipsoid protrusion adds value to solvent accessibility, that may be predicted high in overhanging regions concealing the amino acid in question, yet being inaccessible for considerably large antibody recognition sites. Also, predicting surface area as a metric for solvent accessibility is not compensating for amino acids' specific size. Consequently, the small Glycine will on average have a smaller surface area compared to large amino acids like Tryptophan. Increased probe sizes and amino acid size normalization may thus be improvements for the previously suggested Snowflake prediction pipeline.

Opposed to that, Snowball is amino acid size-independent. Rendering peptides of experimental Class II structures into superimposed predicted structures is a simplistic approach which assumes similar peptide alignments across different Class II proteins' binding grooves. Obviously, this approach will not be reliable for all configurations but it roughly allows identifying exposed regions within a locus. Certainly, including a more robust approach to predict Class II peptide docking improves downstream predictions.

In summary, herein a new method to identify amino acid protrusion of HLA molecules is presented to support the definition of antibody accessibility in HLA epitope definition. Herein the also the Snow algorithm is proposed, that combines surface area and repeated ellipsoid protrusion as two markers of accessibility potential increasing specificity of amino acid mismatch score approaches in HLA matching for transplantation. Studies evaluating the performance in predicting kidney graft loss and immunological events are planned.

### Example 2

### “Snow” Matching Model

Niemann et al. recently proposed the Snowflake amino acid matching score. In brief, Snowflake computes the predicted surface area of donor HLA proteins' amino acid positions. Amino acids exceeding a surface area threshold are considered as being exposed and compared to recipients' self HLA proteins. Exposed amino acids being mismatched with the patients increase the Snowflake score by one. It has been shown that surface area varies within HLA loci or Class which lead to the Snowflake model considering surface area in a protein-specifically [8].

The authors now suggest an improvement named Snow, which combines the Snowflake model with the method according to the invention described herein that applies repeated ellipsoid protrusion ranking of amino acid positions, named “Snowball”. The present method (Snowball) iterates over atoms of HLA protein structure data fitting ellipsoids to substructures of atoms in proximity to the current center atom (in the present example with a 15 Å radius). The atoms' protrusion is ranked based on the ellipsoid's axes with the furthest atom being ranked 1.0 and the atom centered in the ellipsoid being ranked 0.0. The median of all structure's atoms' protrusion ranks is formed and for each residue the maximum of its atomic protrusion medians is considered the amino acid protrusion rank. The Snow algorithm considers amino acid positions exposed, if they exceed both Snowflake (surface area) and Snowball (protrusion) thresholds. Exposed donor amino acids being mismatched with recipient self-HLA increment the Snow score by one.

### Pregnancy Dataset

The previously described pregnancy cohort (PC) of the University Hospital Basel consists of 231 pregnant women who gave live birth between September 2009 and April 2011. [15-18] The study was approved by the local ethics committee. The median age of the mothers was 31 years (Q1=28, Q3=35). Prior immunization events (blood transfusions, transplantations, or miscarriages) were ruled out. High-resolution typing for HLA-A, -B, -C, -DPA1, -DPB1, -DQA1, -DQB1, -DRB1, and -DRB3/4/5 was available for all study participants (i.e. women and children) by means of next-generation HLA sequencing (NGSgo® HLA amplification and library preparation (www.gendx.com, GenDx, Utrecht, the Netherlands), sequencing on Illumina® MiSeq™ (www.illumina.com, Illumina, San Diego, USA)). Maternal HLA antibody specificities were assessed previously, by single antigen beads (LabScreen™ Single HLA Antigen Beads (SAB), OneLambda Thermo Fisher, Canoga Park, CA, USA)) on sera collected between days 1 and 4 after delivery. Antibody data were mapped to the child HLA typing to identify CSA, considering a sample-specific biological cutoff of MFI values above 100 and exceeding the mean of all SABself-HLA mother+ 3 SDs as reported previously [17].

### Kidney Transplantation Dataset

This example used data from the Scientific Registry of Transplant Recipients (SRTR). The SRTR data system includes data on all donor, wait-listed candidates, and transplant recipients in the US, submitted by the members of the Organ Procurement and Transplantation Network (OPTN). The Health Resources and Services Administration (HRSA), U.S. Department of Health and Human Services provides oversight to the activities of the OPTN and SRTR contractors.

The SRTR cohort (SRTRC) considered a subset of 190,598 kidney transplanted patients between 1996 and 2022. Low resolution HLA-A/-B/-DR typings of recipients and donors were used to apply molecular matching methods.

In order to convert the available HLA typing data into protein-level typings, the previously described multiple imputation approach was applied [9,19]. Multi allele codes were converted into antigen-specific candidate lists (https://hml.nmdp.org/MacUI/, IPD-IMGT/HLA version 3.47) [20]. Despite a rather heterogeneous cohort, potential haplotype pairs were fetched solely from the 2011 EURCAU NMDP haplotype dataset [21]. The converted high-resolution haplotype pairs were filtered to a normalized threshold of 1%. For each high-resolution recipient-donor-combination, molecular matching scores were calculated and aggregated by summation and weighting of the respective pair's frequency.

### Reference Epitope Definitions

Eplet definitions of the HLA Eplet Registry (www.epregistry.com.br, version 3.0) [22] have been considered for comparison with Snow scores. PIRCHE-II scores (www.pirche.com, version 3.47) were calculated (reviewed in [23]) via the PIRCHE web service. In the SRTRC, HLA-A, -B, -C, -DRB1 and -DQB1 presented by HLA-DRB1 have been considered for of predicted indirectly recognized HLA epitopes (PIRCHEs) analysis. Following the previously suggested approach of normalizing PIRCHE scores by the respective presenting molecules' predicted peptide binding promiscuity, a corrected PIRCHE score (PIRCHEc) has been calculated. [18] Log transformation of PIRCHE scores has been performed as suggested previously by Lachmann et al. [24]. To this extent, the PIRCHE-II and PIRCHEc scores have been incremented by one preventing infinite values, followed by calculating the natural logarithm.

### Statistical Analysis

Using PC areas under the receiver operator curves (AUC) of the logarithmized Snow scores have been calculated. A pooled dataset was compiled as described previously. [9] To that extent, each mismatch of the PC and its corresponding CSA status was considered with its specific Snow score. Matched alleles were excluded from the analysis. Second order Akaike Information Criterion values (AIC) were calculated for binomial logistic regressions.

In the SRTRC, death-censored graft survival was considered in Cox proportional hazard analyses. Cox proportional hazard assumptions were tested and AICs have been calculated.

All calculations were executed in R software (R 3.6.1, R Foundation for Statistical Computing, Vienna, Austria) using the libraries MuMIn, pROC, survminer, and survival. Furthermore, the libraries ggplot2, akima and rgl were used for visualization purposes.

### Results

In the PC consisting of 231 mother-child pairs, 937 child HLA mismatches have been analyzed. CSA were detected in 97 women (42%) corresponding to 209 mismatches. The median follow-up time within the 190,598 considered kidney transplant cases of the SRTRC was 8.02 years with 52,329 reported graft losses (27%).

### Snowball and Child-Specific Antibodies

In the PC, Snow thresholds were systematically analyzed with Snowflake and Snowball thresholds ranging from 0.00 and 1.00 in 0.01 increments. Linear models predicting CSA were created for individual loci and the pooled data for each of the thresholds and AUCs were calculated as shown in FIG. 15. In the pooled data, an optimal AUC of 0.620 and AIC of 968.77 was found at a Snowflake threshold of 0.02 and a Snowball threshold of 0.38 (Table 1). The sum of median Snow scores at this threshold is 27 compared to 35 at a threshold pair of 0.0/0.0 indicating exclusion of less relevant amino acid mismatches in predicting CSA.

Table 1: Linear regression models of Snowflake (SNF) considering a previously reported threshold of 0.37 and combined Snowflake/Snowball (SNF & SNB/SNF|SNB) models, considering thresholds yielding maximum AUC in the respective outcome metric. Scores were log-transformed. Significance test by Wilcoxon signed rank. OR: odds ratio, CI: confidence interval, AUC: Area under the receiver operator curve, ΔAUC changes of the AUC.

| Predictor | SNF | SNB | Outcome | OR | CI | p | AUC | ΔAUC |
|---|---|---|---|---|---|---|---|---|
| SNF-A | 0.37 | — | CSA-A | 3.78 | 1.90-8.06 | <0.001 | 0.693 | — |
| SNF \| SNB-A | 0.65 | 0.41 | | 4.50 | 2.24-10.10 | <0.001 | 0.718 | +0.025 |
| SNF & SNB-A | 0.10 | 0.39 | | 4.13 | 2.13-8.78 | <0.001 | 0.723 | +0.030 |
| SNF-B | 0.37 | — | CSA-B | 1.72 | 1.04-2.89 | 0.036 | 0.595 | — |
| SNF \| SNB-B | 0.26 | 0.43 | | 2.33 | 1.34-4.22 | 0.004 | 0.635 | +0.040 |
| SNF & SNB-B | 0.02 | 0.43 | | 2.35 | 1.35-4.27 | 0.004 | 0.635 | +0.040 |
| SNF-C | 0.37 | — | CSA-C | 3.36 | 1.39-9.92 | 0.014 | 0.648 | — |
| SNF \| SNB-C | 0.37 | 0.69 | | 2.93 | 1.31-7.69 | 0.016 | 0.634 | −0.010 |
| SNF & SNB-C | 0.37 | 0.50 | | 3.44 | 1.45-9.52 | 0.009 | 0.648 | +0.000 |
| SNF-DRB1 | 0.37 | — | CSA-DRB1 | 2.05 | 1.14-3.81 | 0.019 | 0.603 | — |
| SNF \| SNB- | 0.53 | 0.75 | | 4.57 | 1.12-19.39 | 0.036 | 0.605 | +0.002 |
| DRB1 | | | | | | | | |
| SNF & SNB- | 0.32 | 0.60 | | 2.17 | 1.18-4.12 | 0.015 | 0.609 | +0.006 |
| DRB1 | | | | | | | | |
| SNF-DQB1 | 0.37 | — | CSA-DQ | 2.57 | 1.22-5.55 | 0.014 | 0.635 | — |
| SNF \| SNB- | 1.00 | 0.80 | | 5.61 | 1.80-18.95 | 0.004 | 0.661 | +0.026 |
| DQB1 | | | | | | | | |
| SNF & SNB- | 0.00 | 0.80 | | 5.61 | 1.80-18.95 | 0.004 | 0.661 | +0.026 |
| DQB1 | | | | | | | | |
| SNF | 0.37 | — | CSA pooled | 1.70 | 1.34-2.17 | <0.001 | 0.601 | — |
| SNF \| SNB | 0.07 | 0.62 | | 2.00 | 1.55-2.61 | <0.001 | 0.620 | +0.19 |
| SNF & SNB | 0.02 | 0.38 | | 2.06 | 1.57-2.71 | <0.001 | 0.620 | +0.19 |

### Snow and Kidney Graft Survival

In the SRTRC, Snow thresholds were evaluated. To reduce search space and thus computational runtime, tested Snowflake thresholds ranged from 0.00 to 1.00 considering 0.10 increments. At the minimum AIC, threshold increments were further reduced to 0.02 between 0.30 and 0.50. Analogous to that, Snowball thresholds were selected in a range from 0.00 to 1.00 considering 0.10 increments, where increments were reduced to 0.02 between 0.50 and 0.74. Snow values could be calculated for 190598 cases. Cox proportional hazard models were built for each of the threshold pairs for (A) Snow, (B) Snow and PIRCHE simultaneously, and (C) Snow, PIRCHE and HLA mismatch score simultaneously. Models' AICs are provided in heatmaps (FIG. 16). Optimal AICs for model A were 0.10/0.00, for models B and 0.30/0.68 for Snowflake and Snowball respectively.

For the identified optimal thresholds, detailed results of Cox proportional hazard models are provided in Table 2. A multivariable model of Snow, PIRCHEc and A-B-DR scores yielded best AIC compared to Snow, PIRCHE or PIRCHEc individually or combined. Considering only reported mismatched transplant cases indicates locus-specific impact on predicting graft survival of considered sub-scores (Table 3).

Table 2: Cox proportional hazard models of HLA matching models in the SRTR cohort. Snow was configured with the optimal threshold of 0.30 and 0.68 for Snowflake and Snowball respectively. OR: odds ratio, CI: confidence interval, AIC: Akaike information criterion, z: Wald statistic value.

| Model | Variable | OR | CI | p | z | AIC |
|---|---|---|---|---|---|---|
| Snow | log(Snow + 1) | 1.30 | 1.29-1.32 | <0.001 | 34.78 | 1206029 |
| PIRCHE | log(PIRCHE-II + 1) | 1.15 | 1.14-1.16 | <0.001 | 30.81 | 1206255 |
| PIRCHEc | log(PIRCHEc + 1) | 1.17 | 1.16-1.18 | <0.001 | 32.24 | 1206106 |
| Snow + | log(Snow + 1) | 1.06 | 1.04-1.07 | <0.001 | 8.40 | 1205959 |
| PIRCHE | log(PIRCHE-II + 1) | 1.22 | 1.19-1.24 | <0.001 | 17.25 | |
| Snow + | log(Snow + 1) | 1.18 | 1.15-1.21 | <0.001 | 13.60 | 1205923 |
| PIRCHEc | log(PIRCHEc + 1) | 1.08 | 1.06-1.10 | <0.001 | 10.23 | |
| Snow + | log(Snow + 1) | 1.09 | 1.06-1.12 | <0.001 | 6.50 | 1205764 |
| PIRCHE + | log(PIRCHE-II + 1) | 1.02 | 1.00-1.03 | 0.017 | 2.39 | |
| ABDR | A-B-DR mismatches | 1.07 | 1.06-1.08 | <0.001 | 14.04 | |
| Snow + | log(Snow + 1) | 1.08 | 1.05-1.11 | <0.001 | 5.38 | 1205753 |
| PIRCHEc + | log(PIRCHEc + 1) | 1.03 | 1.02-1.05 | <0.001 | 4.14 | |
| ABDR | A-B-DR mismatches | 1.06 | 1.05-1.07 | <0.001 | 13.14 | |

Table 3: Cox proportional hazard models of molecular matching models in the mismatched cases of the SRTR cohort considering HLA-A, —B and -DRB1 individually. Snow was configured with the optimal threshold of 0.30 and 0.68 for Snowflake and Snowball respectively. OR: odds ratio, CI: confidence interval, z: Wald statistic value.

| Locus | Variable | OR | CI | p | z |
|---|---|---|---|---|---|
| A | log(Snow-A + 1) | 1.09 | 1.07-1.11 | <0.001 | 10.93 |
| (n = 150801) | log(PIRCHE-II-A + 1) | 1.01 | 0.99-1.02 | 0.286 | 1.07 |
| B | log(Snow-B + 1) | 1.03 | 1.01-1.04 | <0.001 | 3.41 |
| (n = 159717) | log(PIRCHE-II-B + 1) | 1.02 | 1.00-1.03 | 0.016 | 2.42 |
| DRB1 | log(Snow-DRB1 + 1) | 1.02 | 1.01-1.04 | 0.015 | 2.43 |
| (n = 145031) | log(PIRCHE-II- | 1.06 | 1.05-1.08 | <0.001 | 7.12 |
| | DRB1 + 1) | | | | |

### Discussion

In the present example, the inventors could show a significant improvement in prediction of child-specific antibodies and graft loss by employing the present method, thereby considering predicted repeated local ellipsoid protrusion (Snowball), and by additionally predicting surface area (Snowflake) to filter for immuno-relevant amino acid mismatches. In this regard, the present approach in the context of the Snow algorithm outperforms the previously presented Snowflake algorithm. By considering pregnancy as a model system for histocompatibility and applying our method side by side to a broad kidney transplant cohort with reduced typing resolution, the inventors provide support for the value of the present approach in the context of the Snow algorithm to predict immunological events.

Thresholds for the Snow approach have been identified in the PC, with a pair of 0.02/0.38 yielding optimal area under the curve (AUCs) for pooled analysis of CSA (FIG. 15). Configuration of Snow to consider amino acid mismatches exceeding both thresholds at once (i.e., Snowflake AND the present method (Snowball)) outperformed configurations of only one of the thresholds being exceeded (i.e. Snowflake OR the present method (Snowball)). Considering amino acid difference [25] of the remaining filtered amino acid positions performed equally well but did not improve predictive performance of the model (data not shown). Consequently, further analyses focused only on amino acid mismatch numbers with concurrent exceedance of Snowflake and the present method (Snowball) values.

With the applied sensitivity analysis in the SRTRC, different optimal thresholds for Snow were identified. Considering Snow as the only predictor for histocompatibility yields a threshold pair of 0.10/0.00. Notably, this configuration is very close to the simplistic amino acid mismatch count (0.00/0.00). When additionally considering PIRCHE and HLA match grade, optimal AICs are reached at a threshold pair of 0.30/0.68 with AICs outperforming single Snow Cox models (FIG. 16). This finding confirms amino acid matching being a good generalist model to predict impact of the antibody pathway and indirect allorecognition pathway simultaneously, due to their inherent co-dependency on the HLA amino acid sequences. However, when the PIRCHE model, which aims on predicting the indirect pathway, is considered next to Snow, Snow thresholds shift it towards being a more specialized predictor for the antibody pathway, in conjunction outperforming predictive performance of amino acid matching. This proves the additive value of considering multiple specialized prediction models for the different pathways of allorecognition simultaneously and supports the theory of linked recognition in transplant immunology.

Interestingly, A-B-DR match grade remains a significant independent predictor of graft survival, despite the sophisticated modeling approaches of the applied molecular matching methods. The domain-specific information contained in the HLA nomenclature and alleles' serology continues being a major factor, confirming earlier results of Unterrainer et al. [26].

Certain limitations of the present example need to be considered. Although well characterized, the PC can only serve as a model system for transplantation. The immunoregulatory processes known to be induced by pregnancy as reviewed by Abu-Raya et al. [27] may differ from transplantation. Opposed to that, the SRTRC is a well-known dataset of organ transplantation, which however only provides low resolution A-B-DRB1 HLA typing information. In our study it has converted with a previously presented multiple imputation approach. [19] The reliability of HLA typing data imputation is still a matter of scientific debate, which also applies to our study. [28-30] Noise introduced by imputation may thus confound predictive performance of our method when applied prospectively. Lacking financial or practical feasibility of retyping historic transplant cases at large scale, certain deviations between imputed and true predicted molecular matching scores were accepted for our retrospective study setup. Back to back analysis in the high-resolution typed PC confirms results of the SRTRC, increasing the confidence in the applied method. Although locus-specific effects of molecular matching scores on kidney graft survival have been observed in the SRTRC, this analysis warrants confirmation in a cohort including HLA-C and -DQB1 typing and locus-specific outcome metrics like measurements of donor-specific HLA antibodies.

In summary, the herein presented evidence shows that the Snow method according to the invention, i.e., amino acid matching based on surface area and protrusion, is a powerful predictor of kidney graft survival. Also, we could show optimal Snow thresholds that outperform amino acid match scores in conjunction with HLA-A-B-DR matching and PIRCHE matching. This supports the hypothesis of linked recognition and the importance of modeling and considering individual pathways of allorecognition simultaneously.

### Example 3

The present example used data from the Scientific Registry of Transplant Recipients (SRTR). The SRTR data system includes data on all donor, wait-listed candidates, and transplant recipients in the US, submitted by the members of the Organ Procurement and Transplantation Network (OPTN). The Health Resources and Services Administration (HRSA), U.S. Department of Health and Human Services provides oversight to the activities of the OPTN and SRTR contractors.

The cohort considered a total of 542,621 kidney transplantation patients (data lock 2022 Dec. 22). Low resolution HLA-A/-B/-DR typings of recipients and donors were used to apply molecular matching methods. Donor age, African American recipient, donor ethnicity, CMV mismatch, donor type and retransplantations have been considered as known potential risk factors for model augmentation. Furthermore, recipient age (Keith et al., 2006) and Tacrolimus-based maintenance therapy (reviewed by Bowman et al., 2008) were considered as known protective factors for model augmentation predicting death-censored graft survival. After data aggregation and filtering for missing data, a total of 400,935 cases were considered in statistical models (FIG. 18).

### Snow Matching Model

The ‘Snow algorithm’ was utilized in this study to estimate the B-cell immunogenicity of mismatched HLA amino acids. The Snow matching algorithm consists of two modules; ‘Snowflake’ and ‘Snowball’. The Snowflake module evaluates the surface area of amino acids by a neural network that is trained on amino acid configurations and their respective surface area identified in publicly available crystal structures complemented by predicted HLA structures (step B.1). Amino acids exceeding a specified surface area threshold are considered exposed (step B.2). Exposed donor amino acids not present in the recipient's self HLA proteins increases the Snowflake score by one. It is worth noting that the Snowflake model takes into account the variability of surface area across HLA proteins, yielding protein-specific surface maps (Niemann et al., 2022 [8]). For the present study, the Snowflake prediction pipeline has been extended to additionally include the loci HLA-DRB1 and -DQB1.

Considering solvent-accessible surface area alone without normalizing for respective amino acid size underestimates accessibility for small amino acids like glycine and overestimates it for large amino acids like tryptophan. To enhance the accuracy of prediction of antibody accessibility, the Snowball module has been suggested. Snowball employs a local ellipsoid protrusion ranking of amino acid positions. During the process, ellipsoids are iteratively being fitted to substructures of atoms found in close proximity (within 15 Å) to the current center atom (step B.3). The protrusion of atoms is subsequently ranked based on the ellipsoid's axes, with the furthest atom receiving a rank of 1.0 and the atom centered in the ellipsoid receiving a rank of 0.0. By calculating the median of protrusion ranks for each structure's atom and determining the maximum of atomic protrusion medians for each residue, the amino acid protrusion rank is defined. The Snow algorithm considers amino acid positions as exposed if they surpass both the Snowflake (surface area) and Snowball (protrusion) thresholds. Exposed donor amino acids mismatched with recipient self-HLA, increment the Snow score by one (step B.4). The Snow matching algorithm is available via www.pirche.com for research purposes.

### Amino Acid Matching

The sum of interlocus Class I and intralocus Class II amino acid configurations of donor HLA not present at the corresponding location in patient HLA was considered as the total number of amino acid mismatches. To that extent, the Snow algorithm has been used without filtering amino acid mismatches by either surface area or protrusion.

### Eplet Matching

Eplet matching has been carried out considering the antibody-verified Eplets listed in the HLA Eplet Registry (www.epregistry.com.br, version 3.0; Duquesnoy et al., 2014 [22]). Interlocus donor HLA-specific Eplets not present in the recipient's self HLA-specific Eplets were considered as mismatched Eplets. The number of such Eplet mismatches is considered as Eplet mismatch score (Kosmoliaptsis et al., 2016 [5]).

### T Cell Epitope Matching

Donor HLA-derived T cell epitopes presented by recipient HLA Class II were calculated by two versions of the PIRCHE-II prediction pipeline; the previously described PIRCHE-II version 3 (v3) and the newly released PIRCHE-II version 4.2 (v4). PIRCHE version 3 (reviewed in Geneugelijk et al., 2018 [23]) counts the number of HLA-derived unique allo core peptides with a binding affinity below 1000 nM as predicted by netMHCIIpan 3.2, denoted as PIRCHE-II score (PIRCHE_II_v3). Following the previously suggested approach of normalizing PIRCHE-II scores by the respective presenting molecules' predicted peptide binding promiscuity, a corrected PIRCHE-II score (PIRCHE_IIc_v3) has been calculated (Niemann et al., 2021 [18]).

PIRCHE version 4 uses a newly developed peptide-HLA binding predictor named Frost for peptide binding by HLA-DRB1. In short, Frost predicts peptide binding using an optimum ensemble of 128 Artificial Neural Networks (ANN) selected from 512 randomly-initialized ANNs that have been trained and tested using binding data from the IEDB database csv export (https://www.iedb.org, accessed 2023/03/20) (Vita et al., 2019 [37]). These ANNs input BLOSUM-62 (Henikoff and Henikoff, 1992) encoded amino acids from a putative binding core, as well as encoded amino acids from the binding groove configuration of the presenting HLA-DRB1, considering two hidden layers of rectified linear units with Adam optimization (Kingma, et al., 2014). Twenty-five relevant binding groove positions were defined in HLA-DRB1 by identifying polymorphic amino acid residues in close proximity (<4 Å) with the bound peptide based on 22 HLA-DRB1 structures from the RCSB PDB (www.rcsb.org) (step A.1) (Berman et al., 2000). The ANNs are trained to minimize error from normalized and allele-specific binding affinities based on IC50 values (step A.2). The final ANN ensembles performed 1000 training iterations, which proved to be the best performing configuration.

In summary, the construction of PIRCHE-II (steps A.1-3) and Snow (steps B.1-4) molecular matching algorithms. Based on HLA crystal structures, amino acid residues forming the binding HLA groove are identified for HLA-DR (step A.1). Experimental peptide HLA binding data curated in the Immune Epitope Database (IEDB, www.iedb.org) trains a neural network predictor (step A.2). Peptide and protein-specific binding affinity is inferred by a trained ensemble of neural networks to support prediction of donor-derived recipient HLA-bound peptides (step A.3). The Snow predictor fetches crystal structures of the Protein Data Bank (RCSB PDB, www.rcsb.org) and augments these with predicted protein structures (step B.1). For these HLA structures, the Snowflake applies a rolling ball algorithm to predict surface area (step B.2), while Snowball uses repeatedly fitted ellipsoids to predict residue protrusion (step B.3). Neural networks trained on surface and protrusion data extrapolate the data to allow protein-specific antibody-accessible amino acid residue matching (step B.4)

For every possible 15-mer peptide derived from HLA-A, -B, -C, -DRB1 and -DQB1, Frost predicts a 9-mer binding core and an allele-specific binding affinity. A binding rank is calculated by comparing the normalized affinity to predicted affinities of random 15-mers derived from human proteins in the Uniprot protein database. The 128 ensemble ANNs vote on a predicted binding core, and a final binding rank is a geometric mean of ranks from those networks which agree on the majority core. Model inference by the selected ensemble allows estimating binding affinity for proteins or peptides that haven't been part of the training data (FIG. 20). The number of unique core peptide binders below a given permille rank threshold is considered as PIRCHE-II score (PIRCHE_II_v4). As PIRv4 incorporates binding affinity ranking, no binding promiscuity post processing is performed.

All PIRCHE-II analyses consider HLA-A, -B, -C, -DRB1 and -DQB1 presented by HLA-DRB1. PIRCHE versions 3 and 4.2 considered HLA protein sequences as reported by IPD-IMGT/HLA version 3.47 and 3.54, respectively (Robinson et al., 2019 [31]). In case of missing exons, sequences were completed by an iterative nearest neighbor approach as previously described (Geneugelijk et al., 2017 [19]).

### Imputation of Protein-Level Typings

In order to convert the available low resolution HLA typing data into protein-level typings, the previously described multiple imputation approach was applied (Geneugelijk et al., 2017 [19]). The reported ethnicity of an individual was mapped to either of the reported populations within the NMDP haplotype dataset (Maiers et al., 2007 [47]). HLA-C and -DQ alleles were imputed based on the available HLA-A, —B and -DR typing. The converted high-resolution haplotype pairs were filtered to a normalized threshold of 1%. For each high-resolution recipient-donor-combination, molecular compatibility scores were calculated and aggregated by summation and weighting of the respective pair's frequency. This process has been automated and integrated as a web-service (www.pirche.com).

### Statistical Models

Death-censored graft survival was considered in two statistical survival models. Firstly, Cox proportional hazard analyses were applied. Cox proportional hazard assumptions were tested and AICs have been calculated on the full dataset. Cox models fitted on 70% of the dataset were formed and the remaining 30% of the data have been used as validation data for AUC calculation. The integral of AUC has been calculated considering the AUC at the 25th, 50th and 75th percentiles of follow-up time considering Uno's suggested AUC estimator using the R package ‘survAUC’ (Uno et al., 2007 [47]; Potapov et al., 2023). To evaluate multicollinearity, variance inflation factors (VIF) have been calculated for each models' variables. Given that Cox models expect linear correlations, but PIRCHE-scores have been shown superlinear, log transformation of PIRCHE-II scores has been performed as suggested previously by Lachmann et al. To that extent, the uncorrected and corrected PIRCHE-II scores have been incremented by one to prevent infinite values, followed by calculating the natural logarithm (PIRCHE_II_v3_log, PIRCHE_IIc_v3_log, PIRCHE_II_v4_log). The same log-transformation has been opportunistically applied to Snow, amino acid and Eplet matching.

Secondly, the recently proposed tree-based gradient boosting system XGBoost was applied (Chen et al., 2016; https://dl.acm.org/doi/10.1145/2939672.2939785). Accelerated failure time models with negative log likelihood models were trained on 70% of the dataset and evaluated on the remaining 30% of the data. Prediction performance was evaluated by Harrel's Concordance Index, Uno's integral of AUC, and variable importance. Hyperparameters were systematically tested for number of trees (16 to 512, following the power of two), maximum tree depth (2 to 12, with one increments). Early-stopping of training was allowed if model performance has not improved for a number of consecutive trees (0.5 times the maximum number of trees). A maximum number of 512 bins per feature has been selected to compensate for the wide range of molecular matching scores. Variable importance has been evaluated by gain metric. Log transformation has not been applied given the non-parametric nature of decision trees.

Model robustness has been considered by ten repeats per model building using different random seeds, which impacts both the initial data split and following probabilistic decisions. The median AIC, AUC and Concordance Indices of the various models were considered for competing model analyses.

Cox models were built and evaluated in R software (R 4.2.2, R Foundation for Statistical Computing, https://www.r-project.org). XGBoost models were built and evaluated in python (Python 3.10, Python Software Foundation, https://www.python.org/).

### Threshold Optimization

Snow and PIRCHE-II thresholds were systematically analyzed, with Snowflake solvent accessibility and Snowball protrusion ranks ranging from 0.00 and 1.00. To limit computational runtime, Snow thresholds were incremented in 0.10 steps, with 0.02 increments between 0.20 and 0.70 (Snowflake) and 0.30 and 0.80. (Snowball), respectively. Threshold tuples of Snow were denoted as e.g. 0.22/0.54, which indicates a mismatched amino acid was considered as solvent accessible if the predicted Snowflake surface area score was above 0.22 and the Snowball protrusion rank was above 54%. PIRCHE ‰ ranks ranged from 5‰ to 500‰ with a superlinear distribution (PIRCHEᵣₐₙₖ=[5, 10, 15, 20, 30, 40, 50, 75, 100, 150, 200, 250, 300, 400, 500]). Threshold triples of Snow and PIRCHE-II additionally denote the binding affinity rank of T-cell epitopes. In the threshold triple of e.g. 0.22/0.54/100, T-cell epitopes were considered as PIRCHE-II if their binding affinity rank was below 100‰ (i.e. 10%).

### Results

From the complete SRTR dataset, 400,935 kidney transplant cases were considered in the SRTR cohort. Transplantations in this cohort were carried out between 1991 and 2022 (median 2010) and had a median follow-up time of 4.85 years (25th percentile: 1.98 years, 75th percentile: 8.97 years) with 76,077 reported graft losses (20%). Descriptive statistics of cohort demographics and distributions of molecular matching metrics are provided in Table 4 and FIG. 19.

Table 4: Demographic data of the 400,925 cases considered in the final histocompatibility analyses.

| Variable | Percentage/Median (IQR) |
|---|---|
| Patient male | 60.57% |
| Living donor transplantation | 33.37% |
| Donor black | 11.91% |
| Recipient black | 24.44% |
| Tacrolimus maintenance | 74.00% |
| CMV mismatch | 16.99% |
| Recipient age | 51 (22) |
| Donor age | 40 (23) |
| First transplants | 86.71% |
| A-B-DR mismatches | 4 (2) |
| PIRCHE-II v3 | 70.28 (58.06) |
| PIRCHE-II v4 | 56.00 (35.60) |
| Snow | 35.26 (26.2) |
| Amino acid mismatches | 47.13 (33.28) |
| Antibody-verified Eplet mismatches | 17.10 (10.59) |

### Threshold Analysis for the SRTR Cohort

Threshold analysis for Snow identified a threshold tuples of 0.00/0.44 yielding optimal AIC (FIG. 25). Notably, this threshold configuration rendered the Snow algorithm relying only on amino acid protrusion. For PIRCHE version 4, the optimal AIC was reached at a binding affinity rank of 300‰. When considering a multivariable model of both Snow and PIRCHE, more restrictive thresholds of the Snow model appeared beneficial. The optimal AIC was reached at the threshold triple 0.28/0.66/300 and improved over the individual models' optimal AIC. Including HLA-A, —B and -DR serological matching to the multivariable model identified a slightly advantageous threshold triple of 0.70/0.66/300. Threshold analysis revealed the previously presented Snowflake algorithm (i.e. Snowball threshold fixed at 0.00) being outperformed by Snow in models combined with PIRCHE and HLA-A, -B and -DR matching.

### Competitive Model Analysis

The competitive model analysis revealed significant correlation of molecular matching scores and graft survival. Cox regression identified PIRCHE-II version 4 as the best univariable histocompatibility metric predicting graft survival (iAUC=0.5436), outperforming PIRCHE-II version 3, A-B-DR, Eplet, amino acid and Snow matching based on AIC (FIG. 4). Log-transformation improved AIC for all molecular matching metrics (FIG. 26). The multivariable model of HLA-A, -B and -DR mismatches outperformed the univariable model of the summed number of mismatches on all three loci. Combining histocompatibility metrics in multivariable models generally outperformed models considering only their respective model components univariably. For example, Eplet matching combined with HLA-A, -B and -DR matching had a lower AIC than either Eplet matching or HLA-A, -B and -DR matching alone. Considering only metrics of histocompatibility, competitive model analysis revealed the optimal AIC for a Cox model considering HLA-A, -B, -DR, Snow and PIRCHE-II version 4 with an iAUC of 0.5382. A slightly improved iAUC was found with the univariable PIRCHE-II version 4 model. Considering Snow and Eplets in multivariable models simultaneously revealed elevated VIF for Eplet scores, suggesting their codependency (FIG. 27).

The clinical reference model (i.e. whether donor and patient are black, Tacrolimus maintenance immunosuppression, patient and donor age, living donor, CMV mismatch and the number of pre-transplants) outperformed histocompatibility models in predicting graft survival based on improved AIC and iAUC of 0.6558. The addition of histocompatibility metrics further improved the model based on AIC yet a slight decrease in iAUC was observed. The optimal AIC was reached by augmenting the clinical reference model with HLA-A, -B, and -DR matching, Snow and PIRCHE (FIG. 28), with very similarAIC in a model that replaced Snow by Eplets.

The iAUC decrease in the histocompatibility-augmented models can be explained by a slightly higher short-term prediction performance of the clinical model at 1.98 years post transplantation. Opposed to that however, an increase in the long-term prediction performance can be observed for the clinical models that include histocompatibility metrics at 8.97 years post transplant (FIG. 29).

XGBoost models were trained and hyperparameters were evaluated systematically considering C index (FIGS. 29-30) and iAUC (FIGS. 31-32). Optimal hyperparameter configuration per model was selected for further analyses with median C indices ranging between 0.5305 for PIRCHE version 3 (depth 2, 64 trees) and 0.5409 for an encompassing model of HLA-A, -B, -DR matching, Snow and PIRCHE (depth 3, 64/32 rounds for optimal C index/iAUC). The model of known clinical risk factors improved further by joining it with all considered histocompatibility metrics (depth 2, 256 trees), which resulted in a C index of 0.6636 and an iAUC of 0.6798 (FIG. 23). Opposed to the Cox models, clinical XGBoost models augmented by histocompatibility metrics improved AUC both short- and long-term (FIG. 33).

Variable importance of the best-performing model indicates all variables contributing to the overall model with living donation being the most important variable, followed by recipient being black, Tacrolimus maintenance, donor age, PIRCHE-II, recipient age, number of previous transplantations, CMV mismatch, HLA-DR match, Snow, donor being black and HLA-B and -A mismatch (least important) (FIG. 24). Notably, although HLA-A and —B are least important in the model by an order of magnitude, their importance is still increased compared to adding a random variable to the model (FIG. 34).

Comparing iAUC of Cox and XGBoost models, XGBoost models appear to have a slightly better performance in terms of iAUC of up to 2.5%. This applies both for the histocompatibility-only models and the combined histocompatibility and clinical parameter models.

Ensembles of Cox and XGBoost models were used to infer risk for all individuals. Patients of the test data set were split into risk quartiles and Kaplan Meier plots of death-censored graft survival were generated (FIG. 25). Given matching by HLA-A, -B and -DR is a coarse scale compared to the fine grained molecular compatibility metrics, dividing patients considering the HLA-A, -B, and -DR matching model into risk quartiles did not yield equisized groups, with denser populated low-compatibility groups (FIGS. 25 A and B).

The number of censoring events in the compatibility groups was very similar for the HLA-A, -B, -DR, Snow and PIRCHE Cox model. Conversely, the Cox model containing known clinical risk factors showed a decreasing number of censoring events with decreasing compatibility. The HLA-A, -B and -DR Cox model's number of censoring events fluctuated between the compatibility strata, which may be explained by the differing group sizes (FIG. 26).

Table 5: Selected Cox proportional hazard models with thresholds yielding lowest model AIC. HR: hazard ratio, CI: confidence interval, z: Wald statistic value

| | | | | | proportional | |
|---|---|---|---|---|---|---|
| | | HR | | | hazards | iAUC/ |
| Model | Variable / Threshold | (CI) | p | z | p | AIC |
| A + | A (0-2) | 1.11 (1.10-1.12) | <0.001 | 17.64 | 0.084 | 0.536 / |
| B + | B (0-2) | 1.05 (1.03-1.06) | <0.001 | 7.15 | 0.521 | 1225565 |
| DR | DR (0-2) | 1.07 (1.06-1.09) | <0.001 | 12.48 | 0.049 | |
| Snow | log(Snow + 1) (SNF > | 1.19 (1.18-1.20) | <0.001 | 33.61 | 0.46 | 0.531 / |
| | 0.01, SNB > 0.00) | | | | | 1225807 |
| PIRCHE-II | log(PIRCHE + 1) (<75‰) | 1.18 (1.17-1.19) | <0.001 | 36.65 | 0.87 | 0.535 / |
| | | | | | | 1225436 |
| Snow + | log(Snow + 1) (SNF > | 1.10 (1.08-1.12) | <0.001 | 11.64 | 0.73 | 0.537 / |
| PIRCHE-II | 0.30, SNB > 0.66) | | | | | 1225369 |
| | log(PIRCHE + 1) (<20‰) | 1.12 (1.11-1.13) | <0.001 | 19.62 | 0.40 | |
| Snow + | log(Snow + 1) (SNF > | 1.02 (0.99-1.05) | 0.157 | 1.41 | 0.29 | 0.538 / |
| PIRCHE-II + | 0.28, SNB > 0.58) | | | | | 1225312 |
| ABv | log(PIRCHE + 1) (<20‰) | 1.11 (1.10-1.12) | <0.001 | 17.48 | 0.75 | |
| Eplets + | log(ABv Eplets + 1) | 1.02 (0.99-1.04) | 0.222 | 1.221 | 0.46 | |
| A + | A (0-2) | 1.06 (1.05-1.07) | <0.001 | 9.48 | 0.01 | |
| B + | B (0-2) | 0.99 (0.98-1.01) | 0.606 | −0.52 | 0.93 | |
| DR | DR (0-2) | 1.03 (1.02-1.04) | <0.001 | 4.35 | 0.19 | |
| Snow + | log(Snow + 1) (SNF > | 1.04 (1.01-1.06) | 0.009 | 2.62 | 0.223 | 0.642 / |
| PIRCHE + | 0.28, SNB > 0.58) | | | | | 1212131 |
| ABv | log(PIRCHE + 1) / <20‰ | 1.05 (1.03-1.06) | <0.001 | 6.87 | 0.762 | |
| Eplets + | log(ABv Eplets + 1) | 1.08 (1.05-1.10) | <0.001 | 5.87 | 0.460 | |
| A + | A (0-2) | 0.98 (0.98-1.00) | 0.010 | −2.59 | 0.005 | |
| B + | B (0-2) | 0.98 (0.96-0.99) | 0.001 | −3.27 | 0.712 | |
| DR + | DR (0-2) | 1.05 (1.03-1.06) | <0.001 | 6.73 | 0.028 | |
| D_black + | D_black (0 = no, | 1.29 (1.26-1.31) | <0.001 | 23.08 | <0.001 | |
| R_black + | 1 = yes) | | | | | |
| on_TAC + | R_black (0 = no, | 1.59 (1.56-1.62) | <0.001 | 53.70 | <0.001 | |
| D_age + | 1 = yes) | | | | | |
| R_age + | on_TAC (0 = no, | 0.66 (0.65-0.67) | <0.001 | −53.93 | <0.001 | |
| LD + | 1 = yes) | | | | | |
| prevTx | D_age (in years) | 1.02 (1.02-1.02) | <0.001 | 65.87 | 0.558 | |
| | R_age (in years) | 0.98 (0.98-0.98) | <0.001 | −78.26 | 0.008 | |
| | LD (0 = no, 1 = yes) | 0.61 (0.60-0.62) | <0.001 | −58.47 | <0.001 | |
| | prevTX (number of TX) | 1.25 (1.22-1.27) | <0.001 | 21.73 | <0.001 | |

### Discussion

The present example applies simultaneous threshold analysis to optimally configure and combine molecular compatibility algorithms designed to predict specific pathways of allorecognition. The present example shows that tuning of these algorithms is dependent on the combination of predictors and suggested optimal configurations for correlating death-censored graft survival after kidney transplantation. Based on these optimal configurations, Cox and XGBoost models were trained on the continuous molecular matching metrics.

The present example four distinct molecular compatibility algorithms were applied: Eplet matching considering antibody-verified Eplets (Duquesnoy et al., 2002 [4] and 2007), amino acid matching restricted by protein-specific amino acid surface area and repeated localized protrusion rank (Snow; Niemann et al., 2024 (in preparation)), prediction of indirectly recognizable HLA-derived T cell epitopes (PIRCHE; Geneugelijk et al., 2018 [23]), and the number amino acid mismatches, which also has been previously shown to predict graft survival (Kosmoliaptsis et al., Human Immunology, 2010). The present results show that all four algorithms have a strong correlation with death-censored graft survival (FIGS. 28 and 30).

Advantageous thresholds for the sole Snow model were identified at a threshold pair of 0.00/0.44 with a performance very similar to amino acid matching. An advantageous PIRCHE configuration was found at a binding rank cutoff of 300‰. When combining Snow and PIRCHE in Cox models, optimal AICs are reached at a Snow threshold pair of 0.28/0.66 and PIRCHE cutoff of 300‰ (FIG. 25). Notably, AICs, C statistics and iAUCs of combined histocompatibility models outperform univariable models of histocompatibility considering both Cox and XGBoost models (FIGS. 28 and 30). The shift in Snow configuration confirms amino acid matching being a good generalist model to predict impact of the antibody pathway and indirect allorecognition pathway simultaneously, due to their inherent co-dependency on the HLA amino acid sequences. Both models Snow and PIRCHE in conjunction outperform predictive performance of amino acid matching. This proves the additive value of considering multiple specialized prediction models for the different pathways of allorecognition simultaneously and supports the theory of linked recognition in transplant immunology. Elevated variance inflation factors for Eplet scores in Cox models combining Snow and Eplets confirms their conceptual and statistical similarity (FIG. 27), suggesting the potential of avoiding considering both parameters simultaneously in the same statistical model. The analysis of known risk factors in kidney transplantation revealed additive value of combining molecular compatibility scores next to conventional HLA matching, both for Cox models and XGBoost models. Highest prediction performance was reached with models considering HLA-A, -B, -DR, PIRCHE, and Snow matching, donor being black, recipient being black, Tacrolimus maintenance, donor age, recipient age, living donor, CMV match, and number of previous transplantations. Despite living donation, recipient being black and maintenance immunosuppression were identified as strongest contributors to a prediction model of pre-transplant parameters in XGBoost models, molecular compatibility has considerable value to the model (FIG. 23 and FIG. 34).

The present data confirms previous reports of indirect T cell epitope matching predicting graft survival in the SRTR dataset (Lemieux et al., 2022) and is in line with reports on European kidney transplantation datasets (Geneugelijk et al., 2015 [16]; Lachmann et al., 2017). Our data confirms reports of Truchot et al., that the performance of various model building frameworks to predict kidney allograft outcome is in the same order of magnitude (Truchot et al., 2023). Opposed to that Katidakis et al. report a slight advantage of neural network-based models in predicting survival after liver transplantation. Cox and XGBoost were used presently only as exemplary statistical frameworks for the purpose of identifying impact of molecular compatibility scores and did not aim to provide a prediction model for clinical purposes. The consistently slightly improved iAUC in XGBoost models compared to Cox models observed in the present experiments can likely be attributed to the non-parametric nature of tree-based learning algorithms, which better maps the non-linear molecular matching metrics to immunologic risk.

Although Cox models' AIC is reduced when adding molecular compatibility scores to the reference prediction model of clinical parameters, the iAUC barely changes (FIG. 21). Evaluating the time-dependent AUC however reveals that long-term prediction improves in models considering histocompatibility, which offsets the better short-term prediction of models absent of histocompatibility (FIG. 22). HLA-A, -B, -DR matching remained a strong predictor of graft survival, despite the sophisticated modeling approaches of the applied molecular matching methods. The domain-specific information contained in the HLA nomenclature and alleles' serology continues being a major factor, confirming earlier results of Unterrainer et al. 2021 with more covariates and non-parametric modeling, its additive value in prediction appears to reduce, however.

One strength of the present example is its consideration of four orthogonal molecular matching algorithms simultaneously in a large scale kidney transplantation cohort. And the results support the advances of the present methods over prior art approaches.

### REFERENCES

1. Stegall M D, Gaston R S, Cosio F G, Matas A. Through a Glass Darkly: Seeking Clarity in Preventing Late Kidney Transplant Failure. JASN. 2015 January; 26(1):20-9. doi: 10.1681/ASN.2014040378.
2. Mayrdorfer M, Liefeldt L, Wu K, Rudolph B, Zhang Q, Friedersdorff F, et al. Exploring the Complexity of Death-Censored Kidney Allograft Failure. JASN. 2021 June; 32(6):1513-26. doi: 10.1681/ASN.2020081215.
3. Kramer C S M, Israeli M, Mulder A, Doxiadis IlN, Haasnoot G W, Heidt S, et al. The long and winding road towards epitope matching in clinical transplantation. Transpl Int. 2019 January; 32(1):16-24. doi: 10.1111/tri.13362.
4. Duquesnoy R J. HLAMatchmaker: a molecularly based algorithm for histocompatibility determination. I. Description of the algorithm. Human Immunology. 2002 May; 63(5):339-52. doi: 10.1016/S0198-8859(02)00382-8.
5. Kosmoliaptsis V, Mallon D H, Chen Y, Bolton E M, Bradley J A, Taylor C J. Alloantibody Responses After Renal Transplant Failure Can Be Better Predicted by Donor-Recipient HLA Amino Acid Sequence and Physicochemical Disparities Than Conventional HLA Matching. Am J Transplant. 2016 July; 16(7):2139-47. doi: 10.1111/ajt.13707.
6. Mallon D H, Kling C, Robb M, Ellinghaus E, Bradley J A, Taylor C J, et al. Predicting Humoral Alloimmunity from Differences in Donor and Recipient HLA Surface Electrostatic Potential. JI. 2018 Dec. 15; 201(12):3780-92. doi: 10.4049/jimmunol.1800683.
7. Kramer C S M, Koster J, Haasnoot G W, Roelen D L, Claas FHJ, Heidt S. HLA-EMMA: A user-friendly tool to analyse HLA class I and class II compatibility on the amino acid level. HLA. 2020 July; 96(1):43-51. doi: 10.1111/tan.13883.
8. Niemann M, Matern B M, Spierings E. Snowflake: A deep learning-based human leukocyte antigen matching algorithm considering allele-specific surface accessibility. Front Immunol. 2022 Jul. 29; 13:937587. doi: 10.3389/fimmu.2022.937587.
9. Niemann M, Strehler Y, Lachmann N, Halleck F, Budde K, Hönger G, et al. Snowflake epitope matching correlates with child-specific antibodies during pregnancy and donor-specific antibodies after kidney transplantation. Front Immunol. 2022 Oct. 28; 13:1005601. doi: 10.3389/fimmu.2022.1005601.
10. Duquesnoy R J, Marrari M. Usefulness of the ElliPro epitope predictor program in defining the repertoire of HLA-ABC eplets. Human Immunology. 2017 July; 78(7-8):481-8. doi: 10.1016/j.humimm.2017.03.005.
11. Ponomarenko J, Bui H H, Li W, Fusseder N, Bourne P E, Sette A, et al. ElliPro: a new structure-based tool for the prediction of antibody epitopes. BMC Bioinformatics. 2008 December; 9(1):514. doi: 10.1186/1471-2105-9-514.
12. R Taylor W, M Thornton J, G Turnell W. An ellipsoidal approximation of protein shape. Journal of Molecular Graphics. 1983 June; 1(2):30-8. doi: 10.1016/0263-7855(83)80001-0.
13. Thornton J M, Edwards M S, Taylor W R, Barlow D J. Location of ‘continuous’ antigenic determinants in the protruding regions of proteins. The EMBO Journal. 1986 February; 5(2):409-13. doi: 10.1002/j.1460-2075.1986.tb04226.x.
14. Matern B M, Mack S J, Osoegawa K, Maiers M, Niemann M, Robinson J, et al. Standard reference sequences for submission of HLA genotyping for the 18th International HLA and Immunogenetics Workshop. HLA. 2021 June; 97(6):512-9. doi: 10.1111/tan.14259.
15. Hönger G, Fornaro I, Granado C, Tiercy J M, Hösli I, Schaub S. Frequency and determinants of pregnancy-induced child-specific sensitization. Am J Transplant. 2013 March; 13(3):746-53. doi: 10.1111/ajt.12048.
16. Geneugelijk K, Hönger G, van Deutekom H W M, Thus KA, Keşmir C, Hösli I, et al. Predicted Indirectly Recognizable HLA Epitopes Presented by HLA-DRB1 Are Related to HLA Antibody Formation During Pregnancy: PIRCHE-II in HLA Antibody Formation. American Journal of Transplantation. 2015 December; 15(12):3112-22. doi: 10.1111/ajt.13508.
17. Hönger G, Niemann M, Schawalder L, Jones J, Heck M R, Pasch L A L, et al. Toward defining the immunogenicity of HLA epitopes: Impact of HLA class I eplets on antibody formation during pregnancy. HLA. 2020 November; 96(5):589-600. doi: 10.1111/tan.14054.
18. Niemann M, Matern B M, Spierings E, Schaub S, Hönger G. Peptides Derived From Mismatched Paternal Human Leukocyte Antigen Predicted to Be Presented by HLA-DRB1, -DRB3/4/5, -DQ, and -DP Induce Child-Specific Antibodies in Pregnant Women. Front Immunol. 2021 Dec. 21; 12:797360. doi: 10.3389/fimmu.2021.797360.
19. Geneugelijk K, Wissing J, Koppenaal D, Niemann M, Spierings E. Computational Approaches to Facilitate Epitope-Based HLA Matching in Solid Organ Transplantation. Journal of Immunology Research. 2017; 2017:1-9. doi: 10.1155/2017/9130879.
20. Bochtler W, Maiers M, Bakker J N A, Baier D M, Hofmann J A, Pingel J, et al. An update to the HLA Nomenclature Guidelines of the World Marrow Donor Association, 2012. Bone Marrow Transplant. 2013 November; 48(11):1387-8. doi: 10.1038/bmt.2013.93.
21. Gragert L, Madbouly A, Freeman J, Maiers M. Six-locus high resolution HLA haplotype frequencies derived from mixed-resolution DNA typing for the entire US donor registry. Human Immunology. 2013 October; 74(10):1313-20. doi: 10.1016/j.humimm.2013.06.025.
22. Duquesnoy R J. Update of the HLA class I eplet database in the website based registry of antibody-defined HLA epitopes: HLA class I eplet database update. Tissue Antigens. 2014 June; 83(6):382-90. doi: 10.1111/tan.12322.
23. Geneugelijk K, Spierings E. Matching donor and recipient based on predicted indirectly recognizable human leucocyte antigen epitopes. Int J Immunogenet. 2018 April; 45(2):41-53. doi: 10.1111/iji.12359.
24. Lachmann N, Niemann M, Reinke P, Budde K, Schmidt D, Halleck F, et al. Donor-Recipient Matching Based on Predicted Indirectly Recognizable HLA Epitopes Independently Predicts the Incidence of De Novo Donor-Specific HLA Antibodies Following Renal Transplantation. Am J Transplant. 2017 December; 17(12):3076-86. doi: 10.1111/ajt.14393.
25. Grantham R. Amino Acid Difference Formula to Help Explain Protein Evolution. Science. 1974 Sep. 6; 185(4154):862-4. doi: 10.1126/science.185.4154.862.
26. Unterrainer C, Döhler B, Niemann M, Lachmann N, Susal C. Can PIRCHE-II Matching Outmatch Traditional HLA Matching?Front Immunol. 2021 Feb. 26; 12:631246. doi: 10.3389/fimmu.2021.631246.
27. Abu-Raya B, Michalski C, Sadarangani M, Lavoie P M. Maternal Immunological Adaptation During Normal Pregnancy. Front Immunol. 2020 Oct. 7; 11:575197. doi: 10.3389/fimmu.2020.575197.
28. Engen R M, Jedraszko A M, Conciatori M A, TamburAR. Substituting imputation of HLA antigens for high-resolution HLA typing: Evaluation of a multiethnic population and implications for clinical decision making in transplantation. Am J Transplant. 2021 January; 21(1):344-52. doi: 10.1111/ajt.16070.
29. D'Souza Y, Ferradji A, Saw C L, Oualkacha K, Richard L, Popradi G, et al. Inaccuracies in epitope repertoire estimations when using multilocus allele-level HLA genotype imputation tools. HLA. 2018 July; 92(1):33-9. doi: 10.1111/tan.13307.
30. Senev A, Emonds M P, Van Sandt V, Lerut E, Coemans M, Sprangers B, et al. Clinical importance of extended second field high-resolution HLA genotyping for kidney transplantation. American Journal of Transplantation. 2020 December; 20(12):3367-78. doi: 10.1111/ajt.15938.
31. Robinson J, Barker D J, Georgiou X, Cooper M A, Flicek P, Marsh S G E. IPD-IMGT/HLA Database. Nucleic Acids Research. 2019 Oct. 31; gkz950. doi: 10.1093/nar/gkz950.
32. Camacho C, Coulouris G, Avagyan V, Ma N, Papadopoulos J, Bealer K, et al. BLAST+: architecture and applications. BMC Bioinformatics. 2009 December; 10(1):421. doi: 10.1186/1471-2105-10-421.
33. Cock P J A, Antao T, Chang J T, Chapman B A, Cox C J, Dalke A, et al. Biopython: freely available Python tools for computational molecular biology and bioinformatics. Bioinformatics. 2009 Jun. 1; 25(11):1422-3. doi: 10.1093/bioinformatics/btp163.
34. Jumper J, Evans R, Pritzel A, Green T, Figurnov M, Ronneberger 0, et al. Highly accurate protein structure prediction with AlphaFold. Nature. 2021 Aug. 26; 596(7873):583-9. doi: 10.1038/s41586-021-03819-2.
35. Varadi M, Anyango S, Deshpande M, Nair S, Natassia C, Yordanova G, et al. AlphaFold Protein Structure Database: massively expanding the structural coverage of protein-sequence space with high-accuracy models. Nucleic Acids Research. 2022 Jan. 7; 50(D1):D439-44. doi: 10.1093/nar/gkab1061.
36. Evans R, O'Neill M, Pritzel A, Antropova N, Senior A, Green T, et al. Protein complex prediction with AlphaFold-Multimer [Internet]. Bioinformatics; 2021 October [cited 2022 Oct. 6]. doi: 10.1101/2021.10.04.463034. Available from: http://biorxiv.org/lookup/doi/10.1101/2021.10.04.463034
37. Vita R, Mahajan S, Overton J A, Dhanda S K, Martini S, Cantrell J R, et al. The Immune Epitope Database (IEDB): 2018 update. Nucleic Acids Res. 2019 Jan. 8; 47(D1):D339-43. doi: 10.1093/nar/gky1006.
38. Abella J, Antunes D, Clementi C, Kavraki L. APE-Gen: A Fast Method for Generating Ensembles of Bound Peptide-MHC Conformations. Molecules. 2019 Mar. 2; 24(5):881. doi: 10.3390/molecules24050881.
39. Golub G H, Van Loan C F. Matrix computations. Fourth edition. Baltimore: The Johns Hopkins University Press; 2013. 756 p. (Johns Hopkins studies in the mathematical sciences)
40. Yury. Ellipsoid fit [Internet]. MATLAB Central File Exchange; [cited 2023 Jan. 18]. Available from: https://www.mathworks.com/matlabcentral/fileexchange/24693-ellipsoid-fit
41. Foley J D, editor. Computer graphics: principles and practice. 2nd ed. in C. Reading, Mass: Addison-Wesley; 1995. 1175 p. (Addison-Wesley systems programming series)
42. Amit A G, Mariuzza R A, Phillips S E V, Poljak R J. Three-Dimensional Structure of an Antigen-Antibody Complex at 2.8 Å Resolution. Science. 1986 Aug. 15; 233(4765):747-53. doi: 10.1126/science.2426778.
43. Sheriff S, Silverton E W, Padlan E A, Cohen G H, Smith-Gill S J, Finzel B C, et al. Three-dimensional structure of an antibody-antigen complex. Proc Natl Acad Sci USA. 1987 November; 84(22):8075-9. doi: 10.1073/pnas.84.22.8075.
44. Schuster M, Paliwal K K. Bidirectional recurrent neural networks. IEEE Trans Signal Process. 1997 November; 45(11):2673-81. doi: 10.1109/78.650093.
45. Kingma D P, Ba J. Adam: A Method for Stochastic Optimization [Internet]. arXiv; 2017 [cited 2022 Jun. 15]. Available from: http://arxiv.org/abs/1412.6980
46. Duquesnoy R J. Update of the HLA class I eplet database in the website based registry of antibody-defined HLA epitopes: HLA class I eplet database update. Tissue Antigens. 2014 June; 83(6):382-90. doi: 10.1111/tan.12322.
47. Maiers M, Gragert L, Klitz W. High-resolution HLA alleles and haplotypes in the United States population. Human Immunology. 2007 September; 68(9):779-88.
48. Uno H, Cai T, Tian L, Wei L J. Evaluating Prediction Rules for t-Year Survivors With Censored Regression Models. Journal of the American Statistical Association. 2007 June; 102(478):527-37.

1. A method for determining an amino acid residue protruding from the surface of a human leukocyte antigen (HLA) protein structure, comprising:
(i) employing an ellipsoid-fitting algorithm to a subset of atoms of an HLA protein structure that are in spatial proximity to each other,
(ii) repeating step (i) for multiple subsets of atoms of the HLA protein structure, thereby creating multiple fitted ellipsoids, and
(iii) determining whether an amino acid of an HLA protein structure is surface protruding by considering the spatial distribution of atoms of any given amino acid residue relative to the position of the multiple fitted ellipsoids.

2. The method according to claim 1, wherein step (i) comprises fitting an ellipsoid to a subset of atoms of the HLA protein structure that are in spatial proximity to each other, wherein the ellipsoid is centered to one atom of said subset of atoms, and wherein the ellipsoid comprises atoms surrounding the atom at the center of the ellipsoid in a radius of 1-100 Å (angstrom).

3. The method according claim 2, wherein step (i) further comprises calculating, within a fitted ellipsoid, a protrusion rank for multiple atoms of the HLA protein structure, wherein the protrusion rank is determined based on the distance of a respective atom from the center of the ellipsoid considering its axes lengths.

4. The method according to claim 3, wherein step (iii) comprises determining whether an amino acid of an HLA protein structure is surface protruding by considering the ellipsoid protrusion ranks of atoms of any given amino acid residue relative to the position of the multiple fitted ellipsoids.

5. The method according to claim 3, comprising:
calculating, for each atom of the HLA protein structure, a protrusion rank and a median value of the protrusion ranks (protrusion rank median) determined in steps (i)-(ii) for the respective atom, and
determining for multiple, amino acid residues of the HLA protein structure a protrusion rank (amino acid protrusion rank), wherein the greatest protrusion rank median of the amino acid's atoms is considered the amino acid protrusion rank.

6. The method according to claim 1, wherein the HLA protein structure and the surface protruding residues comprise (i) an extracellular alpha chain, a beta-2-microglobulin and an HLA-bound peptide structure, in case of HLA Class I proteins, or (ii) the protein's extracellular alpha and beta chains, and an HLA-bound peptide structure, in case of HLA Class II proteins.

7. A method for producing a database of predicted surface-protruding residues for multiple human leukocyte antigen (HLA) proteins, comprising:
a. obtaining one or more HLA protein amino acid sequences, for which a protein structure has been experimentally determined,
b. predicting HLA protein structure of one or more HLA proteins, for which a structure has not been experimentally determined,
c. supplementing the predicted HLA protein structure(s) of b. with an HLA-bound peptide, if the structure of b. does not exhibit an HLA-bound peptide,
d. determining surface-protruding residues of each HLA protein structure of a. and c. using the method of claim 1, and
e. generating a database of surface protruding residues for the HLA proteins of a. and c. and HLA proteins additional to those of a. to c., comprising employing a neural network trained on amino acids sequences of the HLA proteins of a. and c. and the corresponding surface-protruding residues determined in d.

8. The method according to claim 7, wherein the neural network according to e. comprises a long short-term memory bidirectional recurrent neural network, and/or
wherein the method is automated, and/or
wherein the supplementing according to c. comprises:
for predicted structures with known binding peptides, adding said known peptides to the HLA protein structure, and/or
for predicted structures without known binding peptides, adding peptides to the HLA protein structure that are known to bind to an HLA allele with a high degree (the greatest degree) of amino acid sequence identity in extracellular domains to the HLA amino acid sequence of said predicted structure without known binding peptides.

9. The method according to claim 7, wherein the neural network according to step e. comprises the HLA alpha chain (for HLA Class I) or the HLA alpha and beta chains (for HLA Class II) amino acid sequence(s) as an input and the surface protruding residues as an output, and/or
wherein the neural network according to step e. is HLA Class and/or HLA-locus specific, for example comprises multiple surface protruding residues for multiple human leukocyte antigen (HLA) proteins from only HLA Class I proteins, only HLA Class II proteins, or only for alleles selected from the same HLA locus, and/or
wherein the database according to step e. comprises surface protruding residues for essentially all HLA proteins (all HLA alleles) in any given HLA Class.

10. The method according to claim 7, comprising additionally determining one or more predicted solvent accessible surface residues of a human leukocyte antigen (HLA) protein, wherein the predicted solvent-accessible surface residues and protruding amino acid residues of a human leukocyte antigen (HLA) protein indicate a human leukocyte antigen (HLA) B-cell epitope.

11. A method for predicting a human leukocyte antigen (HLA) B-cell epitope that is mismatched between two or more subjects, comprising:
i. obtaining biological samples from the two or more subjects,
ii. determining the amino acid sequences of an HLA protein from the two or more subjects,
iii. determining one or more mismatched amino acids of the HLA protein between two or more subjects,
iv. determining whether said mismatched amino acids are predicted to be protruding from a surface of an HLA structure,
comprising comparing said mismatched amino acids to a database of surface-protruding residues for HLA proteins, wherein the surface-protruding residues of the database are predicted by a neural network trained on amino acids sequences of experimentally determined and predicted HLA protein structures, and on the corresponding residues' surface protrusion,
v. wherein a mismatched amino acid predicted to be surface protruding indicates a human leukocyte antigen (HLA) B-cell epitope that is mismatched between two or more subjects.

12. The method according to claim 11, wherein the database is produced using a method for producing a database of predicted surface-protruding residues for multiple human leukocyte antigen (HLA) proteins, comprising:
a. obtaining one or more HLA protein amino acid sequences, for which a protein structure has been experimentally determined,
b. predicting HLA protein structure of one or more HLA proteins, for which a structure has not been experimentally determined,
c. supplementing the predicted HLA protein structure(s) of b. with an HLA-bound peptide, if the structure of b. does not exhibit an HLA-bound peptide,
d. determining surface-protruding residues of each HLA protein structure of a. and c. using a method for determining an amino acid residue protruding from the surface of a human leukocyte antigen (HLA) protein structure, comprising:
(i) employing an ellipsoid-fitting algorithm to a subset of atoms of an HLA protein structure that are in spatial proximity to each other,
(ii) repeating step (i) for multiple subsets of atoms of the HLA protein structure, thereby creating multiple fitted ellipsoids, and
(iii) determining whether an amino acid of an HLA protein structure is surface protruding by considering the spatial distribution of atoms of any given amino acid residue relative to the position of the multiple fitted ellipsoids, and
(iv) generating a database of surface protruding residues for the HLA proteins of a. and c. and HLA proteins additional to those of a. to c., comprising employing a neural network trained on amino acids sequences of the HLA proteins of a. and c. and the corresponding surface-protruding residues determined in d.

13. The method according to claim 11, wherein a mismatched amino acid predicted to be surface protruding indicates an antibody immune response against the corresponding mismatched HLA protein and/or wherein the method additionally comprises determining one or more predicted solvent accessible surface areas of residues of a human leukocyte antigen (HLA) protein, wherein a mismatched amino acid predicted to be surface-protruding and having solvent accessible surface area indicates an antibody immune response against the corresponding mismatched HLA protein.

14. A method for selecting transplantation material for allogeneic transplantation between subjects with one or more human leukocyte antigen (HLA) mismatches, comprising:
i. assessing whether one or more human leukocyte antigen (HLA) B-cell epitopes are mismatched between said subjects according to the method of claim 11, wherein the number of determined mismatched HLA B-cell epitopes positively correlates with an antibody immune response against a corresponding mismatched HLA protein and/or a likelihood of an adverse immune reaction after transplantation, and
ii. transplanting the material from one of the subjects to the other subject.

15. The method according to claim 14, wherein the transplantation comprises solid organ transplantation (such as kidney transplantation, liver transplantation, heart transplantation, lung transplantation), transplantation of stem cells (such as transplantation of hematopoietic stem-cells (HSCT), cord blood or cord blood cells), or transfusion of blood or cell products (such as platelet transfusion) and/or
wherein the method additionally comprises determining one or more mismatched HLA T-cell epitopes between said subjects.

16. The method according to claim 3, wherein the atom furthest from the center is assigned a protrusion rank of 1 and the atom at the center of the ellipsoid is assigned a protrusion rank of 0.

17. The method according to claim 5, comprising determining for each amino acid residues of the HLA protein structure a protrusion rank (amino acid protrusion rank).

18. The method according to claim 8, wherein the method is automated and does not comprise manual curation of the database according to verified antibody binding.

19. The method according to claim 11, wherein step i. comprises identifying amino acid similarity of an HLA protein of one subject with the same amino acid positions in one or more HLA proteins of another subject.

20. The method according to claim 15, wherein determining one or more mismatched HLA T-cell epitopes between said subjects comprises determining the number of predicted indirectly recognized HLA epitopes (PIRCHEs), wherein said PIRCHEs are recipient- or donor-specific HLA-derived peptides from a mismatched recipient or donor HLA allele, respectively, and are predicted to be presented by an HLA molecule, wherein the number of determined mismatched HLA T-cell epitopes positively correlates with a likelihood of an adverse immune reaction after transplantation.
